(19) United States
(12) Patent Application Publication
(10) Pub. No.: US 2025/0313839 A1
(43) Pub. Date: Oct. 9, 2025

(54) CONJUGATES FOR TISSUE-SPECIFIC OLIGONUCLEOTIDE DELIVERY

(71) Applicant: UNIVERSITY OF MASSACHUSETTS, Westborough, MA (US)

(72) Inventors: Anastasia Khvorova, Westborough, MA (US); Takahiro Nakamura, Ibaraki (JP); Vignesh Narayan Hariharan, Southborough, MA (US); Minwook Shin, Seoul (KR); Ken Yamada, Westborough, MA (US)

(21) Appl. No.: 19/055,377

(22) Filed: Feb. 17, 2025

Related U.S. Application Data
(60) Provisional application No. 63/554,713, filed on Feb. 16, 2024.

(51) Int. Cl. C12N 15/113

(52) U.S. Cl. CPC C12N 15/113; C12N 2310/14; C12N 2310/315; C12N 2310/321; C12N 2310/322; C12N 2310/3515

(57) ABSTRACT

Provided herein are conjugated oligonucleotides including a linker with one or more hydrophobicity- or valency-modulating headgroups Z^{c} having the structure of formula (II):

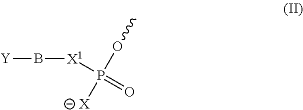

wherein:
Y is selected from the group consisting of —[NR₃]⁺, —[SR₂]⁺, —NR₂, and —CH(NH₂)—CO₂H, wherein each R is independently selected from the group consisting of hydrogen, and optionally substituted C1-C9 alkyl, C1-C9 heteroalkyl, C1-C9 alkenyl, C1-C9 heteroalkenyl, C1-C1 alkynyl, and C1-C9 heteroalkynyl groups;
B is a bridge group selected from the group consisting of substituted or unsubstituted C2-C9 alkyl, C2-C9 heteroalkyl, C2-C9 alkenyl, C2-C9 heteroalkenyl, C2-C9 alkynyl, and C2-C9 heteroalkynyl groups;
X¹ is absent, an oxygen atom, or NH;
X is oxygen, sulfur or borane; useful for RNA interference (RNAi).

### CROSS REFERENCE TO RELATED APPLICATION

This application claims the benefit of U.S. Provisional Patent Application Ser. No. 63/554,713, filed Feb. 16, 2024. The entire content of the above-referenced patent application is incorporated by reference in its entirety herein.

### STATEMENT OF FEDERALLY SPONSORED RESEARCH

This invention was made with government support under Grant Nos. HD086111, GM131839, and OD020012 awarded by the National Institutes of Health. The Government has certain rights in the invention.

### TECHNICAL FIELD

This disclosure relates to novel conjugated oligonucleotides useful for RNA interference (RNAi). The conjugated oligonucleotide provide tunable extrahepatic tissue targeting via defined linker headgroup chemistry.

### BACKGROUND

RNA interference represents a simple and effective tool for inhibiting the function of genes. The promise of RNA interference as a general therapeutic strategy, however, depends on the ability to deliver small RNAs to a wide range of tissues. Currently, small therapeutic RNAs can only be delivered effectively to liver. Safe, effective, and target-tissue-specific delivery of the RNA that directs modulation of gene expression is a critical hurdle in the development of clinical applications for engineered RNA systems. Conjugate-mediated delivery is emerging as the clinically dominant delivery paradigm for siRNAs. There remains a need for targeted delivery to a range of tissues, expanding the clinical utility of this powerful therapeutic platform beyond the liver.

### SUMMARY

In one aspect, the present disclosure provides conjugated oligonucleotides for tissue-specific delivery, such as a conjugated oligonucleotide represented by formula (I):

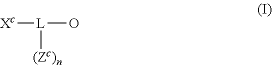

wherein:
O represents one or more oligonucleotides;
L represents a linker;
X^{c} represents a hydrophobic moiety; and
Z^{c} represents a headgroup having the structure of formula (II):

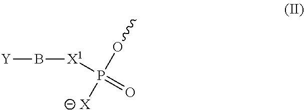

wherein:
Y is selected from the group consisting of —[NR₃]⁺, —[SR₂]⁺, —NR₂, and —CH(NH₂)—CO₂H, wherein each R is independently selected from the group consisting of hydrogen, and optionally substituted C1-C9 alkyl, C1-C9 heteroalkyl, C1-C9 alkenyl, C1-C9 heteroalkenyl, C1-C1 alkynyl, and C1-C9 heteroalkynyl groups;
B is a bridge group selected from the group consisting of substituted or unsubstituted C2-C9 alkyl, C2-C9 heteroalkyl, C2-C9 alkenyl, C2-C9 heteroalkenyl, C2-C9 alkynyl, and C2-C9 heteroalkynyl groups;
X¹ is absent, an oxygen atom, or NL;
X is oxygen, sulfur or borane; and
n is 1 or 2;
wherein when n is 1, Z^{c} is not phosphocholine, phosphoserine, or phosphoethanolamine.

In certain embodiments, B can include one or more substituents independently selected from the group consisting of alkyl, substituted alkyl, alkenyl, substituted alkenyl, halides, —OR′, —NR′R″, —CF₃, —CN, —NO₂, —C₂R′, —SR′, —N₃, —C(═O)NR′R″, —NR′C(O)R″, —C(═O)R′—C(═O)OR′—OC(O)R′, —OC(O)NR′R″, —NR′C(O)OR″, —SO₂R′, —SO₂NR′R″, and —NR′SO₂R″, wherein R′ and R″ are individually hydrogen or C1-C10 alkyl groups.

In certain embodiments, Y can be represented by formula (III)

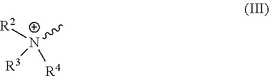

wherein R², R³, and R⁴ can be independently selected from a hydrogen atom and substituted or unsubstituted C1-C10 alkyl, alkenyl, alkynyl, heteroalkyl, heteroalkenyl, or heteroalkynyl groups, wherein each heteroatom can be independently selected from oxygen, nitrogen, silicon, sulfur, and halogen atoms.

In certain embodiments, substituents for R², R³, and R⁴ independently can be selected from the group consisting of alkyl, substituted alkyl, alkenyl, substituted alkenyl, halides, —OR′, —NR′R″, —CF₃, —CN, —NO₂, —C₂R′, —SR′, —N₃, —C(═O)NR′R″, —NR′C(O)R″, —C(═O)R′—C(═O)OR′—OC(O)R′, —O(CR′R″)ᵣC(═O)R′, —O(CR′R″)ᵣNR″C(O)R′, —O(CR′R″)ᵣNR″SO₂R′, —OC(O)NR′R″, —NR′C(O)OR″, —SO₂R′, —SO₂NR′R″, and —NR′SO₂R″, wherein R′ and R″ individually can be hydrogen, C1-C9 alkyl, cycloalkyl, heterocyclyl, aryl, or arylalkyl, and r can be an integer from 1 to 6.

In certain embodiments, R², R³, and R⁴ each, independently, can be a hydrogen atom or an unsubstituted C1-C9 alkyl group. In some embodiments, R², R³, and R⁴ can be methyl groups.

In certain embodiments, L can include an ethylene glycol chain, an alkyl chain, a peptide, RNA, DNA, a phosphate, a phosphonate, a phosphoramidate, an ester, an amide, a carbamate, a triazole, or a combination thereof. In some cases, any carbon or oxygen atom of L can bear a hydroxyl or oxo substituent, an amide, a carbamate, or a combination thereof.

In certain embodiments, L can be connected to O via one or more branch points. In some embodiments, L can be connected to O by one or more spacers, and one or more branch points. In some embodiments, each of the one or more branch points can be a polyvalent organic species or derivative thereof; and each of the one or more spacers, independently, can be selected from the group consisting of an ethylene glycol chain, an alkyl chain, a peptide, RNA, DNA, a phosphate, a phosphonate, a phosphoramidate, an ester, an amide, a triazole, and combinations thereof. In some embodiments, the one or more branch points can be selected from the group consisting of triols, tetrols, tri-carboxylic acids, tetra-carboxylic acids, tertiary amines, triamines, tetramines, and amino acids. In some embodiments, the conjugated oligonucleotide can include 1 to 3 branch points.

In certain embodiments, X^{c} can include one or more fatty acids, steroids, secosteroids, lipids, gangliosides and nucleoside analogs, or endocannabinoids.

In certain embodiments, X^{c} can include a saturated hydrocarbon chain.

In certain embodiments, X^{c} can include an unsaturated hydrocarbon chain having fewer than three double bonds or a polyunsaturated hydrocarbon chain having three or more double bonds.

In certain embodiments, X^{c} can include one or more vitamins, neuromodulatory lipids, omega-3 fatty acids, omega-6 fatty acids, omega-9 fatty acids, conjugated linolenic acids, or saturated fatty acids.

In certain embodiments, X^{c} can include one or more of eicosapentaenoic acid (EPA), docosahexaenoic acid (DHA), cholesterol, myristic acid, palmitic acid and docosanoic acid (DCA).

In certain embodiments, X^{c} is DCA.

In certain embodiments, B can include an unsubstituted C2, C3, C6, or C9 alkyl.

In certain embodiments, X is oxygen.

In certain embodiments, n is 2. In some embodiments, each Z^{c}, independently, can be selected from the group consisting of phosphocholine, choline phosphonate, phosphoethanolamine, (2-aminoethyl)phosphonic acid, phosphoserine, and serine phosphonate. In some embodiments, each Z^{c} is phosphocholine.

In certain embodiments, the conjugated oligonucleotide of Formula (I) includes 2, 3, 4, 6 or 8 oligonucleotides.

In certain embodiments, the one or more oligonucleotides, independently, can be selected from the group consisting of DNA, siRNA, shRNA, antagomiR, miRNA, antisense molecules, gapmers, mixmers, and guide RNA.

In certain embodiments, each oligonucleotide is single-stranded and has a 5′ end and a 3′ end, and at least one of the one or more oligonucleotides is connected to L at the 5′ end or at the 3′ end.

In certain embodiments, the nucleotides at positions 1-6 from the 3′ end, or positions 1-7 from the 3′ end of each oligonucleotide can be connected to adjacent nucleotides via phosphorothioate linkages. In some embodiments, at least one oligonucleotide can include a capping group at the 5′ end. In some embodiments, the capping group can be selected from the group consisting of:

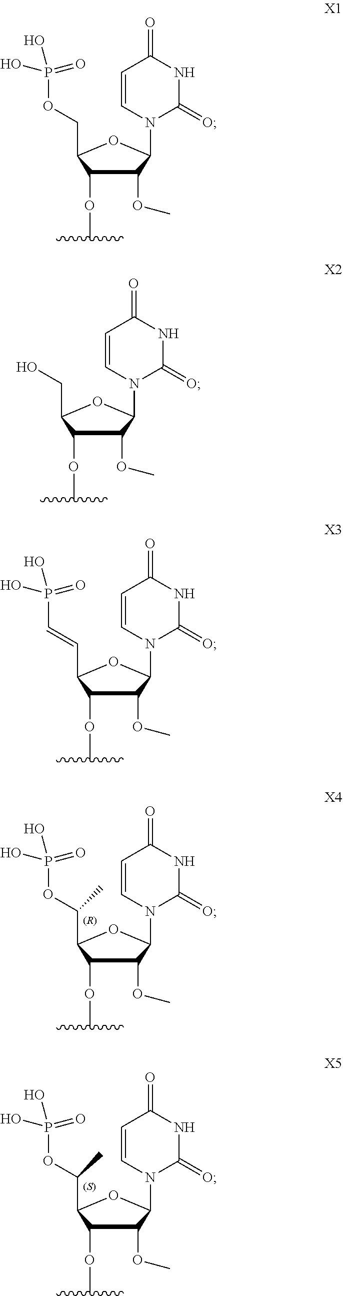

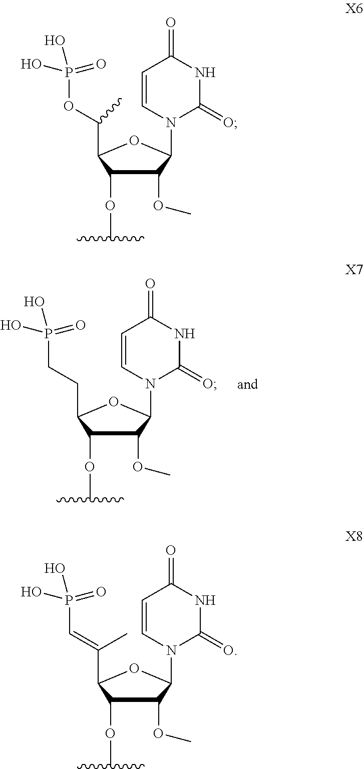

In some embodiments, the capping group is X3.

In certain embodiments, at least one oligonucleotide has complementarity to a target.

In certain embodiments, each oligonucleotide, independently, can include at least contiguous nucleotides.

In certain embodiments, each oligonucleotide can include one or more chemically-modified nucleotides. In some embodiments, the one or more chemically-modified nucleotides can include a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, morpholino nucleotide, a phosphoramidate, or a non-natural base comprising nucleotide.

In certain embodiments, each oligonucleotide can include at least one 2′-O-methyl modified nucleotide and at least one nucleotide comprising a 5′ phosphorothioate group.

In certain embodiments, each oligonucleotide can be at least 50% chemically modified, at least 75% chemically modified, or at least 80% chemically modified.

In certain embodiments, at least one of the one or more oligonucleotides consists of chemically-modified nucleotides.

In certain embodiments, each oligonucleotide is fully chemically modified.

In certain embodiments, O can include one or more double-stranded oligonucleotides, each comprising a sense strand and an antisense strand, wherein the sense strand and the antisense strand each have a 5′ end and a 3′ end. In some embodiments, O can include two to four double-stranded oligonucleotides. In some embodiments, the sense strand and the antisense strand can each include one or more chemically-modified nucleotides. In some embodiments, the sense strand and the antisense strand each consist of chemically-modified nucleotides. In some embodiments, at least one of the sense strand and the antisense strand comprise a region of alternating 2′-methoxy-nucleotides and 2′-fluoro-nucleotides. In some embodiments, the nucleotides of the antisense strand can be connected via phosphodiester or phosphorothioate linkages. In some embodiments, the nucleotides of the sense strand can be connected via phosphodiester or phosphorothioate linkages. In some embodiments, the nucleotides at positions 1 and 2 from the 3′ end of the sense strand can be connected to adjacent nucleotides via phosphorothioate linkages. In some embodiments, the nucleotides at positions 1 and 2 from the 5′ end of the sense strand can be connected to adjacent nucleotides via phosphorothioate linkages. In some embodiments, the nucleotides at positions 1 and 2 from the 5′ end of the sense and antisense strands can be connected to adjacent nucleotides via phosphorothioate linkages. In some embodiments, the one or more double-stranded oligonucleotides comprise at least one modified internucleotide linkage of formula (IV):

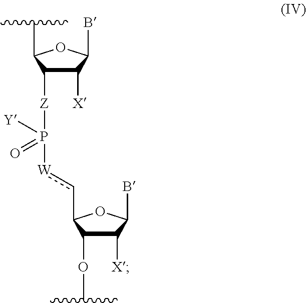

wherein:
   B′ is a base pairing moiety;
   W is selected from the group consisting of O, OCH₂, OCH, CH₂, and CH, or W is O or O(CH);
   X′ is selected from the group consisting of H, halo, hydroxy, OR, F, SH, SR, NR²₂ and C1-C6-ALKOXY;
   Y′ is selected from the group consisting of O⁻, OH, OR, OR², NH⁻, NH₂, NR²₂, BH₃, S⁻, R¹, and SH;
   Z is selected from the group consisting of O, CH₂ or O(CH₂)ₙ;
   R is a protecting group;
   R¹ is alkyl, allyl or aryl; and
   R² is alkyl, allyl or aryl,
   n is 1 to 10, and [symbol] is an optional double bond.

In some embodiments, protecting group R can be selected from the group consisting of dimethoxytrityl (DMTr), succinate, tert-butyl dimethylsilyl (TBDMS), benzoyl (Bz), benzyl (Bn), methoxyethoxymethyl ether (MOM), methoxybenzyl ether (PMB), methylthiomethyl ether, pivaloyl (Piv), tetrahydropyranyl (THP), tetrahydrofuranyl (THF), trityl (Trt), triisopropylsilyl (TIPS), tert-butyldiphenylsilyl (TBDPS), and acetate. In some embodiments, each of the one or more double-stranded oligonucleotides can be connected to L at the 3′ end or at the 5′ end of the sense strand or the antisense strand, connected to a branch point at the 3′ end or at the 5′ end of the sense strand or the antisense strand, or connected to a spacer at the 3′ end or at the 5′ end of the sense strand or the antisense strand. In some embodiments, the branch point can be a polyvalent organic species or derivative thereof. In some embodiments, the branch point can be selected from the group consisting of triols, tetrols, tri-carboxylic acids, tetra-carboxylic acids, tertiary amines, triamines, tetramines, and amino acids; and the spacer can be an ethylene glycol chain, an alkyl chain, a peptide, RNA, DNA, a phosphate, a phosphonate, a phosphoramidate, an ester, an amide, a triazole, or a combination thereof. In some embodiments, the antisense strand can include at least 16, at least 17, at least 18, at least 19, or at least 20 contiguous nucleotides, and can have complementarity to a target.

In some embodiments, the conjugated oligonucleotide includes a double stranded oligonucleotide where: (1) the antisense strand comprises at least 16 contiguous nucleotides, a 5′ end, a 3′ end and has complementarity to a target; (2) the sense strand comprises at least 15 contiguous nucleotides, a 5′ end, a 3′ end, and has homology with a target; and (3) a portion of the antisense strand is complementary to a portion of the sense strand.

In some embodiments, O can be attached to L, a branch point, or a spacer via the sense strand. In some embodiments, the branch point can be a polyvalent organic species or derivative thereof, optionally selected from the group consisting of triols, tetrols, tri-carboxylic acids, tetra-carboxylic acids, tertiary amines, triamines, tetramines, and amino acids; and the spacer can be an ethylene glycol chain, an alkyl chain, a peptide, RNA, DNA, a phosphate, a phosphonate, a phosphoramidate, an ester, an amide, a triazole, or a combination thereof. In some embodiments, the nucleotides at positions 2 and 14 from the 5′ end of the sense strand are 2′-methoxy-ribonucleotides.

In some embodiments, the conjugated oligonucleotide includes a double-stranded oligonucleotide where: (1) the antisense strand includes alternating 2′-methoxy-ribonucleotides and 2′-fluoro-ribonucleotides, wherein each nucleotide is a 2′-methoxy-ribonucleotide or a 2′-fluoro-ribonucleotide; and the nucleotides at positions 2 and 14 from the 5′ end of the antisense strand are not 2′-methoxy-ribonucleotides; (2) the sense strand includes alternating 2′-methoxy-ribonucleotides and 2′-fluoro-ribonucleotides, wherein each nucleotide is a 2′-methoxy-ribonucleotide or a 2′-fluoro-ribonucleotide; and the nucleotides at positions 2 and 14 from the 5′ end of the sense strand are 2′-methoxy-ribonucleotides; (3) the nucleotides of the antisense strand can be connected to adjacent nucleotides via phosphodiester or phosphorothioate linkages, wherein the nucleotides at positions 1-6 from the 3′ end, or positions 1-7 from the 3′ end can be connected to adjacent nucleotides via phosphorothioate linkages; and (4) the nucleotides of the sense strand can be connected to adjacent nucleotides via phosphodiester or phosphorothioate linkages, wherein the nucleotides at positions 1 and 2 from the 3′ end are connected to adjacent nucleotides via phosphorothioate linkages.

In some embodiments, the antisense strand comprises a capping group at the 5′ end, optionally the capping group is one of X1-X8 as described above. In some embodiments, the capping group is X3.

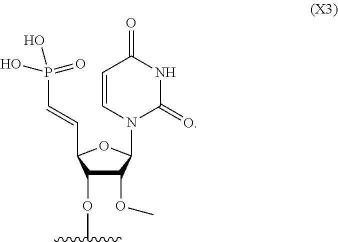

In some embodiments, the antisense strand can have 3-7 more nucleotides than the sense strand. In some embodiments, the one or more double-stranded oligonucleotides can include a duplex region having a length of from 11 to 16 base pairs. In some embodiments, the antisense strand can have perfect complementarity to the target. In some embodiments, the sense strand can have complete homology with the target. In some embodiments, the target is mammalian or viral mRNA. In some embodiments, the target is an intronic region of the mammalian or viral mRNA.

In some embodiments, the one or more double-stranded oligonucleotides are double-stranded RNA (dsRNA).

In certain embodiments, L can be selected from the group consisting of:

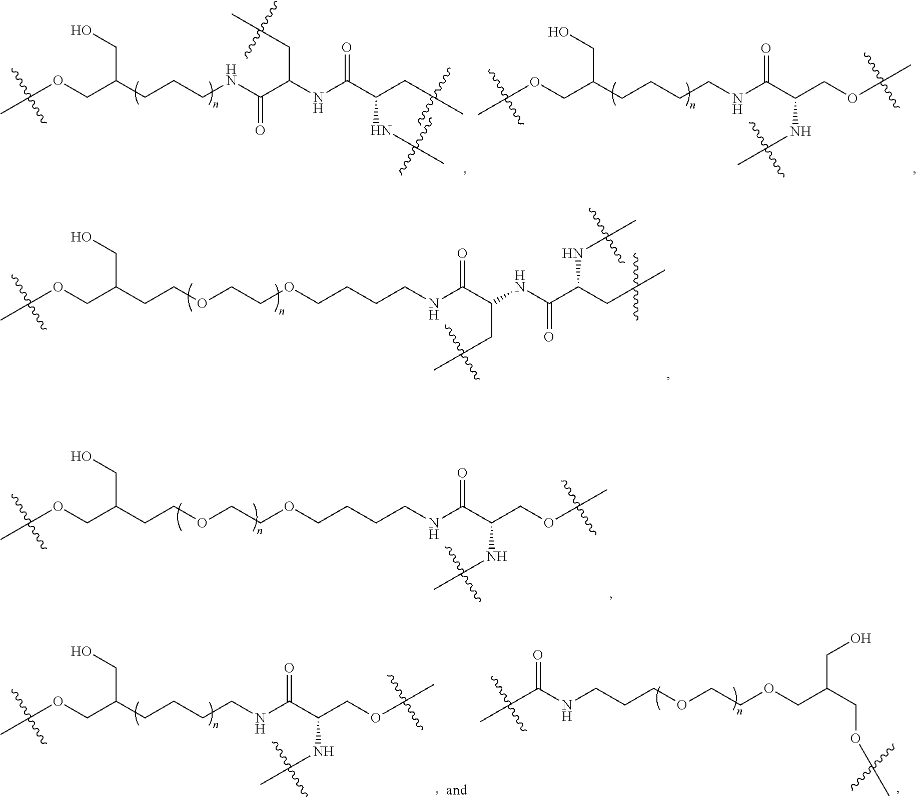

wherein n is 0, 1, 2, 3, 4, or 5.

In some embodiments, n is 1 and L is:

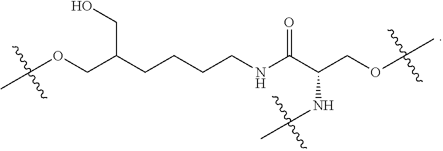 [SMILES: *N[C@@H](CO*)C(=O)NCCCCC(CO)CO*]

In some embodiments, n is 2 and L is:

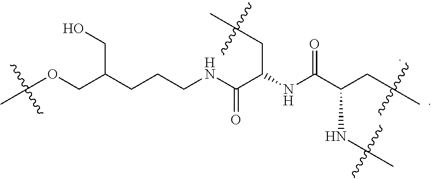

In certain embodiments of Formula (I), O is a siRNA; L is:

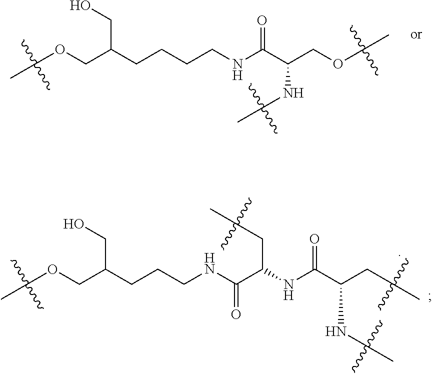

X is DCA;
and Z^{c} is:

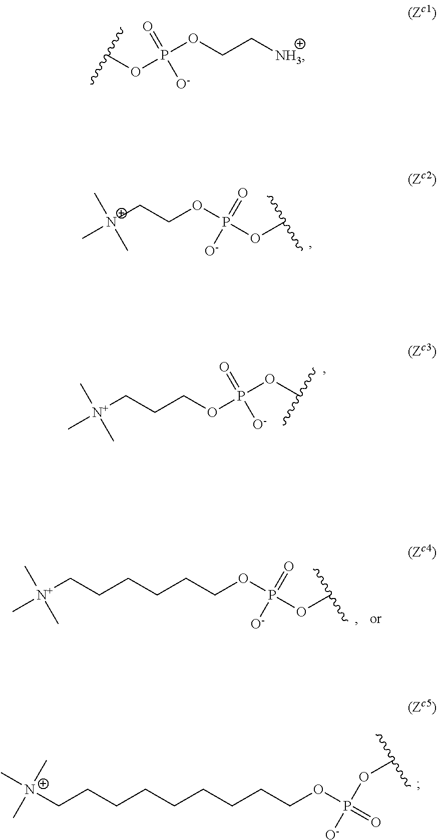

n is 1 or 2;
wherein when n is 1, Z^{c} is not Z^{c1} or Z^{c2}.

In some embodiments, the siRNA includes: (1) an antisense strand comprising alternating 2′-methoxy-ribonucleotides and 2′-fluoro-ribonucleotides, wherein each nucleotide is a 2′-methoxy-ribonucleotide or a 2′-fluoro-ribonucleotide; and the nucleotides at positions 2 and 14 from the 5′ end of the antisense strand are not 2′-methoxy-ribonucleotides; (2) a sense strand comprising alternating 2′-methoxy-ribonucleotides and 2′-fluoro-ribonucleotides, wherein each nucleotide is a 2′-methoxy-ribonucleotide or a 2′-fluoro-ribonucleotide; and the nucleotides at positions 2 and 14 from the 5′ end of the sense strand are 2′-methoxy-ribonucleotides; (3) the nucleotides of the antisense strand are connected to adjacent nucleotides via phosphodiester or phosphorothioate linkages, wherein the nucleotides at positions 1-6 from the 3′ end, or positions 1-7 from the 3′ end are connected to adjacent nucleotides via phosphorothioate linkages; and (4) the nucleotides of the sense strand are connected to adjacent nucleotides via phosphodiester or phosphorothioate linkages, wherein the nucleotides at positions 1 and 2 from the 3′ end are connected to adjacent nucleotides via phosphorothioate linkages. In some embodiments, the antisense strand comprises a capping group at the 5′ end, wherein the capping group is:

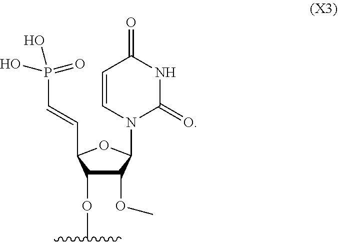

In another aspect, the present disclosure provides a pharmaceutical composition comprising a therapeutically effective amount of one or more conjugated oligonucleotides according to the aspect described above (e.g., Formula (I)), or any one of the embodiments thereof; and a pharmaceutically acceptable carrier.

In another aspect, the present disclosure provides a method for selectively delivering a conjugated oligonucleotide of one or more conjugated oligonucleotides according to Formula (I), or any one of the embodiments thereof to a particular organ in a patient, comprising administering said conjugated oligonucleotide or the pharmaceutical composition of the aspect above to the patient, wherein the conjugated oligonucleotide selectively accumulates in one or more of heart, kidneys, muscle, lung, urinary bladder, pancreas, duodenum, spleen, adrenal gland, reproductive, extra-embryonic, and fat tissue of the patient.

In certain embodiments, the conjugated oligonucleotide selectively accumulates in at least heart, muscle or lung.

In another aspect, the present disclosure provides a method for tissue-specific silencing of a therapeutic target in a subject in need thereof, comprising administering the conjugated oligonucleotide according to Formula (I), or any one of the embodiments thereof or the pharmaceutical composition of the aspect above to the subject, whereby the conjugated oligonucleotide selectively accumulates in one or more of heart, kidneys, muscle, lung, urinary bladder, pancreas, duodenum, spleen, adrenal gland, reproductive, extra-embryonic, and fat tissue in the subject.

In certain embodiments, the conjugated oligonucleotide selectively accumulates in at least heart, muscle or lung.

In another aspect, the present disclosure provides a multivalent conjugated oligonucleotide of formula (I_{A}):

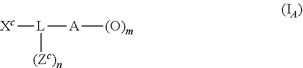

wherein:
O represents an oligonucleotide;
m is at least two;
L represents a linker;
A represents one or more branching points, one or more spacers, or a combination thereof;
X^{c} represents a hydrophobic moiety; and
Z^{c} represents a headgroup represented by formula (II):

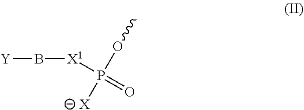

wherein:
Y is selected from the group consisting of —[NR₃]⁺, —[SR₂]⁺, —NR₂, and —CH(NH₂)—CO₂H, wherein each R is, independently, selected from the group consisting of hydrogen, and optionally substituted C1-C9 alkyl, C1-C9 heteroalkyl, C1-C9 alkenyl, C1-C9 heteroalkenyl, C1-C1 alkynyl, and C1-C9 heteroalkynyl groups;
B is a bridge group selected from the group consisting of substituted or unsubstituted C2-C9 alkyl, C2-C9 heteroalkyl, C2-C9 alkenyl, C2-C9 heteroalkenyl, C2-C9 alkynyl, and C2-C9 heteroalkynyl groups;
X¹ is absent, an oxygen atom, or NH;
X is oxygen, sulfur or borane; and
n is 1 or 2;
wherein when n is 1, Z^{c} is not phosphocholine, phosphoserine, or phosphoethanolamine.

In certain embodiments, m is 2, 3 or 4.

In certain embodiments, L can include an ethylene glycol chain, an alkyl chain, a peptide, RNA, DNA, a phosphate, a phosphonate, a phosphoramidate, an ester, an amide, a carbamate, a triazole, or a combination thereof; wherein any carbon or oxygen atom of L optionally bears a hydroxyl or oxo substituent, an amide, a carbamate, or a combination thereof.

In certain embodiments, L can be selected from the group consisting of:

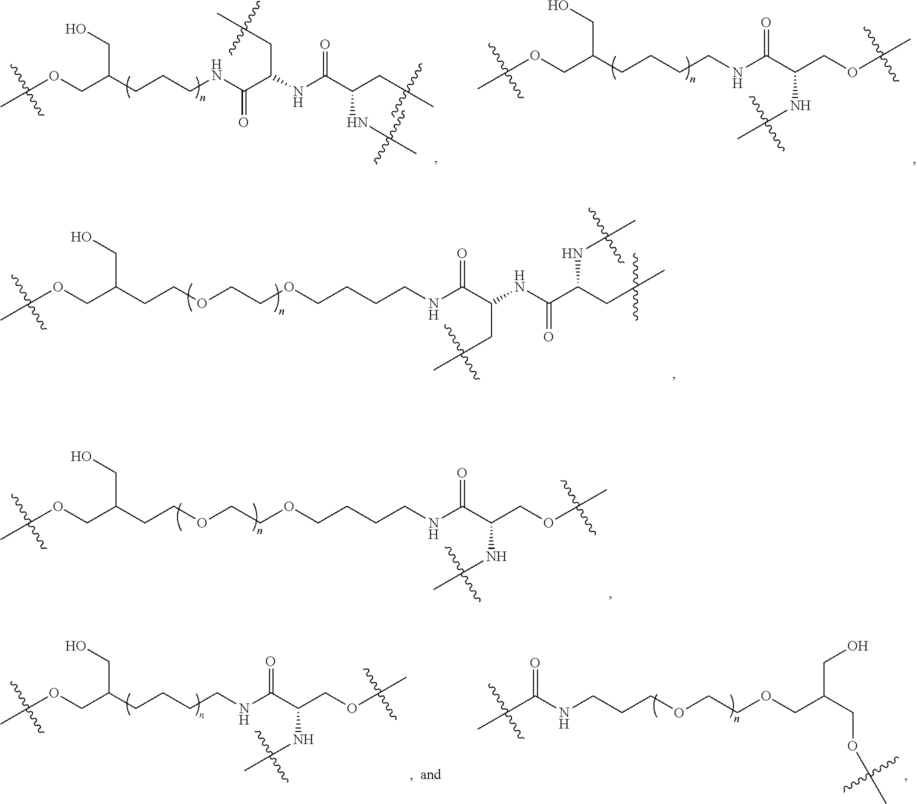

wherein n is 0, 1, 2, 3, 4, or 5.

In certain embodiments, n is 1 and L is:

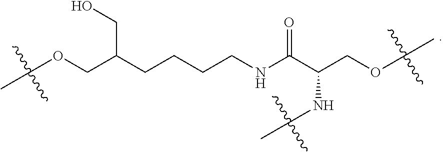 [SMILES: *N[C@@H](CO*)C(=O)NCCCCC(CO)CO*]

In certain embodiments, n is 2 and L is:

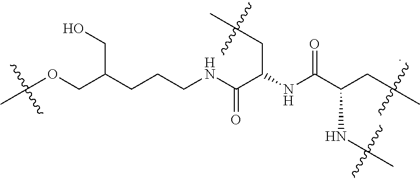

In certain embodiments, each branching point can be a polyvalent organic species or derivative thereof.

In certain embodiments, each branching point can be selected from the group consisting of triols, tetrols, tri-carboxylic acids, tetra-carboxylic acids, tertiary amines, triamines, tetramines, and amino acids.

In certain embodiments, variable A can include 1 to 3 branching points.

In certain embodiments, variable A can include one or more spacers.

In certain embodiments, each of the one or more spacers, independently, can be selected from an ethylene glycol chain, an alkyl chain, a peptide, RNA, DNA, a phosphate, a phosphonate, a phosphoramidate, an ester, an amide, a triazole, and combinations thereof.

In certain embodiments, B includes one or more substituents, independently, selected from the group consisting of alkyl, substituted alkyl, alkenyl, substituted alkenyl, halides, —OR′, —NR′R″, —CF₃, —CN, —NO₂, —C₂R′, —SR′, —N₃, —C(═O)NR′R″, —NR′C(O)R″, —C(═O)R′ C(═O)OR′—OC(O)R′, —OC(O)NR′R″, —NR′C(O)OR″, —SO₂R′, —SO₂NR′R″, and —NR′SO₂R″, wherein R′ and R″ are individually hydrogen or C1-C10 alkyl groups.

In certain embodiments, Y is represented by formula (III) as described above, wherein R², R³, and R⁴ are, independently, selected from a hydrogen atom and substituted or unsubstituted C1-C10 alkyl, alkenyl, alkynyl, heteroalkyl, heteroalkenyl, or heteroalkynyl groups, wherein each heteroatom is, independently, selected from oxygen, nitrogen, silicon, sulfur, aid halogen atoms.

In certain embodiments, substituents for R², R³, and R⁴, independently, can be selected from the group consisting of alkyl, substituted alkyl, alkenyl, substituted alkenyl, halides, —OR′, —NR′R″, —CF₃, —CN, —NO₂, —C₂R′, —SR′, —N₃, —C(═O)NR′R″, —NR′C(O)R″, —C(═O)R′ C(═O)OR′—OC(O)R′, —O(CR′R″)ᵣC(═O)R′, O(CR′R″)ᵣNR″C(O)R′, —O(CR′R″)ᵣNR″SO₂R′, —OC(O)NR′R″, —NR′C(O)OR″, —SO₂R′, —SO₂NR′R″, and —NR′SO₂R″, wherein R′ and R″ are individually hydrogen, C1-C9 alkyl, cycloalkyl, heterocyclyl, aryl, or arylalkyl, and r is an integer from 1 to 6.

In certain embodiments, R², R³, and R⁴, independently, can be selected from hydrogen and an unsubstituted C1-C9 alkyl group.

In certain embodiments, each of R², R³, and R⁴ are hydrogen atoms or methyl groups.

In certain embodiments, X^{c} can include one or more fatty acids, steroids, secosteroids, lipids, gangliosides and nucleoside analogs, or endocannabinoids.

In certain embodiments, X^{c} can include a saturated hydrocarbon chain.

In certain embodiments, X^{c} can include an unsaturated hydrocarbon chain having fewer than three double bonds or a polyunsaturated hydrocarbon chain having three or more double bonds.

In certain embodiments, X^{c} can include one or more vitamins, neuromodulatory lipids, omega-3 fatty acids, omega-6 fatty acids, omega-9 fatty acids, conjugated linolenic acids, or saturated fatty acids.

In certain embodiments, X^{c} can include one or more of eicosapentaenoic acid (EPA), docosahexaenoic acid (DHA), cholesterol, myristic acid, palmitic acid and docosanoic acid (DCA).

In certain embodiments, X^{c} is DCA.

In certain embodiments, B is a substituted or unsubstituted alkyl group having 2 to 9 carbon atoms.

In certain embodiments, B is an unsubstituted C2, C3, C6, or C9 alkyl group. In some embodiments, X is oxygen.

In certain embodiments, n is 2. In some embodiments, each Z^{c} can be, independently, selected from phosphocholine, choline phosphonate, phosphoethanolamine, (2-aminoethyl)phosphonic acid, phosphoserine, and serine phosphonate. In some embodiments, each Z^{c} is phosphocholine.

In certain embodiments, the one or more oligonucleotides can be, independently, selected from the group consisting of DNA, siRNA, shRNA, antagomiR, miRNA, antisense molecules, gapmers, mixmers, and guide RNA.

In certain embodiments, the oligonucleotide is a single-stranded oligonucleotide having a 5′ end and a 3′ end, wherein O is connected to variable A at the 5′ end or at the 3′ end.

In some embodiments, the nucleotides at positions 1-6 from the 3′ end, or positions 1-7 from the 3′ end of the single-stranded oligonucleotide are connected to adjacent nucleotides via phosphorothioate linkages.

In some embodiments, the single-stranded oligonucleotide comprises a capping group at the 5′ end. In some embodiments, the capping group is selected from the group consisting of X1-X8, described above. In some embodiments, the capping group is X3. In some embodiments, the single-stranded oligonucleotide has complementarity to a target.

In some embodiments, the single-stranded oligonucleotide, independently, at each occurrence comprises at least 15 contiguous nucleotides.

In some embodiments, the single-stranded oligonucleotide, independently, at each occurrence comprises one or more chemically-modified nucleotides. In some embodiments, the one or more chemically-modified nucleotides comprises a 2′-deoxy-2′-fluoro modified nucleotide, a 2′-deoxy-modified nucleotide, a locked nucleotide, an abasic nucleotide, a 2′-amino-modified nucleotide, a 2′-alkyl-modified nucleotide, morpholino nucleotide, a phosphoramidate, or a non-natural base comprising nucleotide. In some embodiments, the single-stranded oligonucleotide, independently, at each occurrence comprises at least one 2′-O-methyl modified nucleotide and at least one nucleotide comprising a 5′ phosphorothioate group. In some embodiments, the single-stranded oligonucleotide, independently, at each occurrence is at least 50% chemically modified, at least 75% chemically modified, or at least 80% chemically modified. In some embodiments, the single-stranded oligonucleotide consists of chemically-modified nucleotides. In some embodiments, the single-stranded oligonucleotide is fully chemically modified.

In certain embodiments, the oligonucleotide is a double-stranded oligonucleotide comprising a sense strand and an antisense strand, wherein the sense strand and the antisense strand each have a 5′ end and a 3′ end. In some embodiments, formula (I_{A}) Comprises 2-4 double-stranded oligonucleotides.

In some embodiments, the sense strand and the antisense strand each comprise one or more chemically-modified nucleotides. In some embodiments, the sense strand and the antisense strand each consist of chemically-modified nucleotides. In some embodiments, at least one of the sense strand and the antisense strand comprise a region of alternating 2′-methoxy-nucleotides and 2′-fluoro-nucleotides.

In some embodiments, the nucleotides of the antisense strand are connected via phosphodiester or phosphorothioate linkages. In some embodiments, the nucleotides of the sense strand are connected via phosphodiester or phosphorothioate linkages. In some embodiments, the nucleotides at positions 1 and 2 from the 3′ end of the sense strand are connected to adjacent nucleotides via phosphorothioate linkages. In some embodiments, the nucleotides at positions 1 and 2 from the 5′ end of the sense strand are connected to adjacent nucleotides via phosphorothioate linkages. In some embodiments, the nucleotides at positions 1 and 2 from the 5′ end of the sense and antisense strands are connected to adjacent nucleotides via phosphorothioate linkages.

In some embodiments, the double-stranded oligonucleotide comprises at least one modified internucleotide wherein the double-stranded oligonucleotide comprises at least one modified internucleotide linkage of formula (IV), as described above. In some embodiments, variable R of formula (IV) includes a protecting group selected from the group consisting of dimethoxytrityl (DMTr), succinate, tert-butyl dimethylsilyl (TBDMS), benzoyl (Bz), benzyl (Bn), methoxyethoxymethyl ether (MOM), methoxybenzyl ether (PMB), methylthiomethyl ether, pivaloyl (Piv), tetrahydropyranyl (THP), tetrahydrofuranyl (THF), trityl (Trt), triisopropylsilyl (TIPS), tert-butyldiphenylsilyl (TBDPS), and acetate.

In some embodiments, the double-stranded oligonucleotide, independently, at each occurrence is connected to variable A at the 3′ end or at the 5′ end of the sense strand or the antisense strand.

In some embodiments, the antisense strand comprises at least 16, at least 17, at least 18, at least 19, or at least 20 contiguous nucleotides, and has complementarity to a target.

In some embodiments, the double-stranded oligonucleotide has: (1) an antisense strand has at least 16 contiguous nucleotides, a 5′ end, a 3′ end and complementarity to a target; (2) the sense strand has at least 15 contiguous nucleotides, a 5′ end, a 3′ end, and has homology with a target; and (3) a portion of the antisense strand that is complementary to a portion of the sense strand.

In some embodiments, O at each occurrence is attached to variable A via the sense strand.

In some embodiments, the nucleotides at positions 2 and 14 from the 5′ end of the sense strand are 2′-methoxy-ribonucleotides.

In some embodiments, the double-stranded oligonucleotide includes: (1) an antisense strand including a region of alternating 2′-methoxy-ribonucleotides and 2′-fluoro-ribonucleotides, wherein each nucleotide is a 2′-methoxy-ribonucleotide or a 2′-fluoro-ribonucleotide; and the nucleotides at positions 2 and 14 from the 5′ end of the antisense strand are not 2′-methoxy-ribonucleotides; (2) a sense strand including a region of alternating 2′-methoxy-ribonucleotides and 2′-fluoro-ribonucleotides, wherein each nucleotide is a 2′-methoxy-ribonucleotide or a 2′-fluoro-ribonucleotide; and the nucleotides at positions 2 and 14 from the 5′ end of the sense strand are 2′-methoxy-ribonucleotides; (3) the nucleotides of the antisense strand connected to adjacent nucleotides via phosphodiester or phosphorothioate linkages, wherein the nucleotides at positions 1-6 from the 3′ end, or positions 1-7 from the 3′ end are connected to adjacent nucleotides via phosphorothioate linkages; and (4) the nucleotides of the sense strand connected to adjacent nucleotides via phosphodiester or phosphorothioate linkages, wherein the nucleotides at positions 1 and 2 from the 3′ end are connected to adjacent nucleotides via phosphorothioate linkages.

In some embodiments, the antisense strand comprises a capping group at the 5′ end. In some embodiments, the capping group is selected from the group consisting of X1-X8 described above. In some embodiments, the capping group is X3 described above.

In some embodiments, the antisense strand has 3-7 more nucleotides than the sense strand.

In some embodiments, the double-stranded oligonucleotide comprises a duplex region having a length of from 11 to 16 base pairs.

In some embodiments, the antisense strand has perfect complementarity to the target. In some embodiments, the sense strand has complete homology with the target. In some embodiments, the target is mammalian or viral mRNA. In some embodiments, the target is an intronic region of the mammalian or viral mRNA.

In some embodiments, the double-stranded oligonucleotide is double-stranded RNA (dsRNA).

In another aspect, the present disclosure provides a pharmaceutical composition comprising a therapeutically effective amount of one or more multivalent conjugated oligonucleotides according Formula (I_{A}), or any embodiment thereof described above; and a pharmaceutically acceptable carrier.

In another aspect, the present disclosure provides a method for selectively delivering a multivalent conjugated oligonucleotide according Formula (I_{A}), or any embodiment thereof described above to a particular organ in a patient, comprising administering the multivalent conjugated oligonucleotide or the pharmaceutical composition of the aspect above to the patient, wherein the conjugated oligonucleotide selectively accumulates in one or more of heart, kidneys, muscle, lung, urinary bladder, pancreas, duodenum, spleen, adrenal gland, reproductive, extra-embryonic, and fat tissue of the patient. In some embodiments, the conjugated oligonucleotide selectively accumulates in at least heart, muscle or lung.

In another aspect, the present disclosure provides a method for tissue-specific silencing of a therapeutic target in a subject in need thereof, comprising administering the multivalent conjugated oligonucleotide according Formula (I_{A}), or any embodiment thereof described above or the pharmaceutical composition of the aspect above to the subject, whereby the conjugated oligonucleotide selectively accumulates in one or more of heart, kidneys, muscle, lung, urinary bladder, pancreas, duodenum, spleen, adrenal gland, reproductive, extra-embryonic and fat tissue in the subject, optionally in at least heart, muscle or lung. In some embodiments, the conjugated oligonucleotide selectively accumulates in at least heart, muscle or lung.

### BRIEF DESCRIPTION OF THE DRAWINGS

FIGS. 1A-1B depict exemplary headgroup variants for use altering the hydrophobicity of docosanoic acid conjugated siRNA (DCA-siRNA), according to one or more embodiments of the present disclosure: (FIG. 1A) is a schematic of conjugate headgroup variants used in the working examples below and (FIG. 1B) shows High Performance Liquid Chromatography (HPLC) spectra of DCA (blue), DCA having a phosphocholine headgroup (PC-C2-DCA (red)), DCA having a C3 alkyl chain (“linker”) between the phosphate moiety and the trimethyl ammonium moiety (referred to as “choline” or “choline moiety” herein) (PC-C3-DCA (light green)), DCA having a C6 alkyl chain between the phosphate moiety and the choline moiety (PC-C6-DCA (pink)), DCA having a C9 alkyl chain between the phosphate moiety and the choline moiety (PC-C9-DCA (gold)), DCA having a two phosphocholine headgroups (PC-PC-DCA (purple)) and DCA having a phosphoethanolamine headgroup (PEth-DCA (dark green)), each of which are conjugated to siRNA^{htt} sense strands.

FIGS. 2A-2C depict results demonstrating the effect of phosphate-choline linker length on extrahepatic tissue accumulation of siRNA: (FIG. 2A) shows siRNA^{cd47} accumulation measured using the peptide nucleic acid hybridization assay, (FIG. 2B) shows relative cd47 mRNA expression levels measured by the QUANTIGENE 2.0 assay (Bayer) and (FIG. 2C) shows relative htt mRNA expression levels measured by the QUANTIGENE 2.0 assay in various tissues of female FVB mice injected with 20 mg/kg of conjugate variants. Conjugate with the highest accumulation in each tissue represented as colored bars. Bars represent the mean±s.e.m and of individual animals (n=5). Statistical significance computed using one-way ANOVA with DCA as the comparator in (A) or two-way ANOVA with Šidák's multiple comparisons test with NTC as the comparator in (B) and (C) (*-p<0.05, **-p<0.01, ***-p<0.001, ****-p<0.001).

FIGS. 3A-3C depict results demonstrating the effect of increasing the charge lipid ratio of the conjugate on tissue accumulation: (FIG. 3A) shows siRNA^{cd47} accumulation measured using the peptide nucleic acid hybridization assay, and (FIG. 3B) shows relative cd47 mRNA expression levels measured by the QUANTIGENE 2.0 assay and (FIG. 3C) relative htt mRNA expression levels measured by the QUANTIGENE 2.0 assay in various tissues of female FVB mice injected with 20 mg/kg of conjugate variants. Conjugate with the highest accumulation in each tissue represented as colored bars. Bars represent the mean±s.e.m and of individual animals (n=5). Statistical significance computed using one-way ANOVA with DCA as the comparator in (A) or two-way ANOVA with Šidák's multiple comparisons test with NTC as the comparator in (B) and (C) (*-p<0.05, **-p<0.01, ***-p<0.001, ****-p<0.001). Statistical comparisons within siRNA^{cd47} groups in (B) and within siRNA^{htt} groups in (C) performed using one-way ANOVA using DCA as the comparator ($-p<0.05, $$-p<0.01) or PC-C2-DCA as the comparator (##-p<0.01).

FIG. 4 shows scheme S1 for synthesis of choline p-toluenesulfonate derivatives 3a-c.

FIG. 5 shows scheme S2 for synthesis of phosphatidylcholine derivatives 7a-c for PC (C3, C6, and C9)-DCA Controlled Pore Glass (CPG) synthesis.

FIG. 6 shows scheme S3 for synthesis of precursor 7d for the CPG immobilized with ethanolamine-DCA.

FIG. 7 shows scheme S4 for synthesis of CPGs immobilized with various lipid conjugates.

FIG. 8 shows scheme S5 for synthesis of PC-PC-DCA immobilized CPG.

FIGS. 9A-9B show NMR characterization of choline p-toluenesulfonate derivative 3b shown in FIG. 4: (FIG. 9A) ¹H NMR (500 MHz, DMSO-d₆) and (FIG. 9B) ¹³C NMR (100 MHz, DMSO-d₆).

FIGS. 10A-10B show NMR characterization of choline p-toluenesulfonate derivative 3c shown in FIG. 4: (FIG. 10A) ¹H NMR (500 MHz, DMSO-d₆) and (FIG. 10B) ¹³C NMR (100 MHz, DMSO-d₆).

FIGS. 11A-11C show NMR characterization of a synthesis intermediate shown in FIG. 5: (FIG. 11A) ¹H NMR (500 MHz, CDCl₃); (FIG. 11B) ¹³C NMR (100 MHz, CDCl₃); and (FIG. 11C) ³¹P NMR (161 MHz, CDCl₃).

FIGS. 12A-12C show NMR characterization of a synthesis intermediate shown in FIG. 5: (FIG. 12A) ¹H NMR (500 MHz, CDCl₃); (FIG. 12B) ¹³C NMR (100 MHz, CDCl₃); and (FIG. 12C) ³¹P NMR (161 MHz, CDCl₃).

FIGS. 13A-13C show NMR characterization of a synthesis intermediate shown in FIG. 5: (FIG. 13A) ¹H NMR (500 MHz, CDCl₃); (FIG. 13B) ¹³C NMR (100 MHz, CDCl₃); and (FIG. 13C) ³¹P NMR (161 MHz, CDCl₃).

FIGS. 14A-14C show NMR characterization of a synthesis intermediate shown in FIG. 6: (FIG. 14A) ¹H NMR (500 MHz, CDCl₃); (FIG. 14B) ¹³C NMR (100 MHz, CDCl₃); and (FIG. 14C) ³¹P NMR (161 MHz, CDCl₃).

FIGS. 15A-15C show NMR characterization of phosphatidylcholine derivative 7a shown in FIG. 7: (FIG. 15A) ¹H NMR (500 MHz, MeOD); (FIG. 15B) ¹³C NMR (100 MHz, MeOD); and (FIG. 15C) ³¹P NMR (161 MHz, MeOD).

FIGS. 16A-16C show NMR characterization of phosphatidylcholine derivative 7b shown in FIG. 7: (FIG. 16A) ¹H NMR (500 MHz, DMSO-d₆); (FIG. 16B) ¹³C NMR (100 MHz, DMSO-d₆); and (FIG. 16C)³¹P NMR (161 MHz, DMSO-d₆).

FIGS. 17A-17C show NMR characterization of phosphatidylcholine derivative 7c shown in FIG. 7: (FIG. 17A) ¹H NMR (500 MHz, DMSO-d₆); (FIG. 17B) ¹³C NMR (100 MHz, DMSO-d₆); and (FIG. 17C)³¹P NMR (161 MHz, DMSO-d₆).

FIGS. 18A-18C show NMR characterization of ethanolamine moiety shown in FIG. 6: (FIG. 18A) ¹H NMR (500 MHz, CDCl₃); (FIG. 18B) ¹³C NMR (100 MHz, CDCl₃); and (FIG. 18C)³¹P NMR (161 MHz, CDCl₃).

### DETAILED DESCRIPTION

The present disclosure relates to conjugates engineered to selectively deliver oligonucleotides, such as therapeutic oligonucleotides, to a range of tissues, including heart, kidneys, muscle, lung, urinary bladder, pancreas, duodenum, spleen, adrenal gland, and fat. The conjugates described herein promote simple, efficient, non-toxic delivery of oligonucleotides, and promote potent silencing of therapeutic targets in a range of tissues in vivo.

Provided herein is a chemistry platform for conjugated oligonucleotides characterized by a headgroup architecture, the modulation of which permits tunable distribution of the conjugated oligonucleotide in one or more extrahepatic tissues. The optimized conjugates show improved distribution, tissue-specific uptake, and efficacy as compared to corresponding DCA conjugates and/or DCA-PC conjugates in several tissues, including heart, muscle, kidney, pancreas, urinary bladder, spleen, lung, adrenal gland, and fat, for example.

In certain aspects, the oligonucleotide conjugates of the present disclosure were identified through a process involving: (1) providing a fully metabolically stable scaffolds (no RNA left); (2) conjugating to hydrophobic moieties which are biologically known to exhibit extrahepatic accumulation (e.g., DCA) via linkers with hydrophobicity- or valency-modulating headgroups; and (3) and screening distribution and efficacy in vivo as compared to known DCA and DCA-PC conjugates. Optimizing linker headgroup chemistry provides for tailored tissue-specific RNAi delivery.

### Definitions

Unless otherwise defined herein, scientific and technical terms used herein have the meanings that are commonly understood by those of ordinary skill in the art. In the event of any latent ambiguity, definitions provided herein take precedent over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

So that the present disclosure may be more readily understood, certain terms are first defined.

The term “nucleoside” refers to a molecule having a purine or pyrimidine base covalently linked to a ribose or deoxyribose sugar. Exemplary nucleosides include adenosine, guanosine, cytidine, uridine and thymidine. Additional exemplary nucleosides include inosine, 1-methyl inosine, pseudouridine, 5,6-dihydrouridine, ribothymidine, 2N-methylguanosine and N2,N2-dimethylguanosine (also referred to as “rare” nucleosides). The term “nucleotide” refers to a nucleoside having one or more phosphate groups joined in ester linkages to the sugar moiety. Exemplary nucleotides include nucleoside monophosphates, diphosphates and triphosphates. The terms “polynucleotide” and “nucleic acid molecule” are used interchangeably herein and refer to a polymer of nucleotides joined together by a phosphodiester or phosphorothioate linkage between 5′ and 3′ carbon atoms.

The term “RNA” or “RNA molecule” or “ribonucleic acid molecule” refers to a polymer of ribonucleotides (e.g., 2, 3, 4, 5, 10, 15, 20, 25, 30, or more ribonucleotides). The term “DNA” or “DNA molecule” or “deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively) or multi-stranded (e.g., double stranded, i.e., dsRNA and dsDNA, respectively). “mRNA” or “messenger RNA” is single-stranded RNA that specifies the amino acid sequence of one or more polypeptide chains. This information is translated during protein synthesis when ribosomes bind to the mRNA.

As used herein, the term “small interfering RNA” (“siRNA”) (also referred to in the art as “short interfering RNAs”) refers to an RNA (or RNA analog) comprising between about 10-50 nucleotides (or nucleotide analogs), which is capable of directing or mediating RNA interference. In certain embodiments, a siRNA comprises between about 15-30 nucleotides or nucleotide analogs, or between about 16-25 nucleotides (or nucleotide analogs), or between about 18-23 nucleotides (or nucleotide analogs), or between about 19-22 nucleotides (or nucleotide analogs) (e.g., 19, 20, 21 or 22 nucleotides or nucleotide analogs). The term “short” siRNA refers to a siRNA comprising about 21 nucleotides (or nucleotide analogs), for example, 19, 20, 21 or 22 nucleotides. The term “long” siRNA refers to a siRNA comprising about 24-25 nucleotides, for example, 23, 24, 25 or 26 nucleotides. Short siRNAs may, in some instances, include fewer than 19 nucleotides, e.g., 16, 17 or 18 nucleotides, provided that the shorter siRNA retains the ability to mediate RNAi. Likewise, long siRNAs may, in some instances, include more than 26 nucleotides, provided that the longer siRNA retains the ability to mediate RNAi absent further processing, e.g., enzymatic processing, to a short siRNA.

The term “nucleotide analog” or “altered nucleotide” or “modified nucleotide” refers to a non-standard nucleotide, including non-naturally occurring ribonucleotides or deoxyribonucleotides. Exemplary nucleotide analogs are modified at any position so as to alter certain chemical properties of the nucleotide yet retain the ability of the nucleotide analog to perform its intended function. Examples of positions of the nucleotide, which may be derivatized include: the 5 position, e.g., 5-(2-amino)propyl uridine, 5-bromo uridine, 5-propyne uridine, 5-propenyl uridine, etc.; the 6 position, e.g., 6-(2-amino)propyl uridine; and the 8-position for adenosine and/or guanosines, e.g., 8-bromo guanosine, 8-chloro guanosine, 8-fluoroguanosine, etc. Nucleotide analogs also include deaza nucleotides, e.g., 7-deaza-adenosine; O- and N-modified (e.g., alkylated, e.g., N6-methyl adenosine, or as otherwise known in the art) nucleotides; and other heterocyclically modified nucleotide analogs, such as those described in Herdewijn, Antisense Nucleic Acid Drug Dev., 2000 Aug. 10(4):297-310.

Nucleotide analogs may also comprise modifications to the sugar portion of the nucleotides. For example, the 2′ OH-group may be replaced by a group selected from H, OR, R, F, Cl, Br, I, SH, SR, NH₂, NHR, NR₂, or COOR, wherein R is substituted or unsubstituted C1-C6 alkyl, alkenyl, alkynyl, aryl, etc. Other possible modifications include those described in U.S. Pat. Nos. 5,858,988, and 6,291,438.

The phosphate group of the nucleotide may also be modified, e.g., by substituting one or more of the oxygens of the phosphate group with sulfur (e.g., phosphorothioates), or by making other substitutions, which allow the nucleotide to perform its intended function, such as described in, for example, Eckstein, Antisense Nucleic Acid Drug Dev. 2000 Apr. 10(2):117-21, Rusckowski et al. Antisense Nucleic Acid Drug Dev. 2000 Oct. 10(5):333-45, Stein, Antisense Nucleic Acid Drug Dev. 2001 Oct. 11(5): 317-25, Vorobjev et al. Antisense Nucleic Acid Drug Dev. 2001 Apr. 11(2):77-85, and U.S. Pat. No. 5,684,143. Certain of the above-referenced modifications (e.g., phosphate group modifications) decrease the rate of hydrolysis of, for example, polynucleotides comprising said analogs in vivo or in vitro.

The term “oligonucleotide” refers to a short polymer of nucleotides and/or nucleotide analogs.

The term “RNA analog” refers to a polynucleotide (e.g., a chemically synthesized polynucleotide) having at least one altered or modified nucleotide as compared to a corresponding unaltered or unmodified RNA, but retaining the same or similar nature or function as the corresponding unaltered or unmodified RNA. As discussed above, the oligonucleotides may be linked with linkages, which result in a lower rate of hydrolysis of the RNA analog as compared to an RNA molecule with phosphodiester linkages. For example, the nucleotides of the analog may comprise methylenediol, ethylene diol, oxymethylthio, oxyethylthio, oxycarbonyloxy, phosphorodiamidate, phosphoramidate, and/or phosphorothioate linkages. Some RNA analogues include sugar- and/or backbone-modified ribonucleotides and/or deoxyribonucleotides. Such alterations or modifications can further include addition of non-nucleotide material, such as to the end(s) of the RNA or internally (at one or more nucleotides of the RNA). An RNA analog need only be sufficiently similar to natural RNA that it has the ability to mediate RNA interference.

As used herein, the term “RNA interference” (“RNAi”) refers to a selective intracellular degradation of RNA. RNAi occurs in cells naturally to remove foreign RNAs (e.g., viral RNAs). Natural RNAi proceeds via fragments cleaved from free dsRNA, which direct the degradative mechanism to other similar RNA sequences. Alternatively, RNAi can be initiated by the hand of man, for example, to silence the expression of target genes.

An RNAi agent, e.g., an dsRNA, having a strand, which includes a “sequence sufficiently complementary to a target mRNA sequence to direct target-specific RNA interference (RNAi)” means that the strand has a sequence sufficient to trigger the destruction of the target mRNA by the RNAi machinery or process.

As used herein, the term “isolated RNA” (e.g., “isolated siRNA” or “isolated siRNA precursor”) refers to RNA molecules, which are substantially free of other cellular material, or culture medium when produced by recombinant techniques, or substantially free of chemical precursors or other chemicals when chemically synthesized.

As used herein, the term “RNA silencing” refers to a group of sequence-specific regulatory mechanisms (e.g. RNA interference (RNAi), transcriptional gene silencing (TGS), post-transcriptional gene silencing (PTGS), quelling, co-suppression, and translational repression) mediated by RNA molecules, which result in the inhibition or “silencing” of the expression of a corresponding protein-coding gene. RNA silencing has been observed in many types of organisms, including plants, animals, and fungi.

As used herein, “headgroup” refers to a substituent of a linker joining one or more oligonucleotides to a hydrophobic moiety. For example, a headgroup of the present disclosure can include a hydrophilic moiety attached to the linker via a phosphate-containing functional group. a headgroup can vary in composition and includes various functional groups, such as choline, ethanolamine, serine, and derivatives thereof.

As used herein, “bridge group” refers to ligand connecting two or more atoms. A bridge group of the present disclosure can be polyatomic, vary in composition and length, and include one or more functional groups. A headgroup can include a bridge group connecting a hydrophilic moiety and phosphate-containing functional group thereof.

As used herein, the term “multivalent conjugate” refers to a conjugated scaffold having at least two oligonucleotides attached, such as a di-valent, tri-valent or tetra-valent conjugate comprising 2, 3, or 4 conjugated oligonucleotides, respectively.

The term “in vitro” has its art recognized meaning, e.g., involving purified reagents or extracts, e.g., cell extracts. The term “in vivo” also has its art recognized meaning, e.g., involving living cells, e.g., immortalized cells, primary cells, cell lines, and/or cells in an organism.

As used herein, the term “target gene” is a gene whose expression is to be substantially inhibited or “silenced.” Similarly, a “target RNA” is a gene product that may be targeted for inhibition by the double stranded RNAs of the disclosure. This silencing can be achieved by RNA silencing, e.g., by cleaving the mRNA of the target gene or translational repression of the target gene. The term “non-target gene” is a gene whose expression is not to be substantially silenced. In one embodiment, the polynucleotide sequences of the target and non-target gene (e.g. mRNA encoded by the target and non-target genes) can differ by one or more nucleotides. In another embodiment, the target and non-target genes can differ by one or more polymorphisms (e.g., Single Nucleotide Polymorphisms or SNPs). In another embodiment, the target and non-target genes can share less than 100% sequence identity. In another embodiment, the non-target gene may be a homologue (e.g. an orthologue or paralogue) of the target gene.

As used herein, the term “antisense strand” of a double-stranded oligonucleotide e.g., a dsRNA or siRNA, refers to a strand that is substantially complementary to a section of about 10-50 nucleotides, e.g., about 15-30, 16-25, 18-23 or 19-22 nucleotides of the mRNA of the gene targeted for silencing. The antisense strand or first strand has sequence sufficiently complementary to the desired target mRNA sequence to direct target-specific silencing, e.g., complementarity sufficient to trigger the destruction of the desired target mRNA by the RNAi machinery or process (RNAi interference) or complementarity sufficient to trigger translational repression of the desired target mRNA.

The term “sense strand” or “second strand” of a double-stranded oligonucleotide, e.g., an siRNA or dsRNA, refers to a strand that is complementary to the antisense strand or first strand. Antisense and sense strands can also be referred to as first or second strands, the first or second strand having complementarity to the target sequence and the respective second or first strand having complementarity to said first or second strand. An miRNA duplex intermediate or siRNA-like duplexes include an miRNA strand having sufficient complementarity to a section of about 10-50 nucleotides of the mRNA of the gene targeted for silencing and a miRNA* strand having sufficient complementarity to form a duplex with the miRNA strand.

As used herein, the term “guide strand” refers to a strand of a double-stranded oligonucleotide, e.g., an antisense strand of a dsRNA or siRNA that enters into the RISC complex and directs cleavage of the target mRNA.

As used herein, the “5′ end,” as in the 5′ end of an antisense strand, refers to the 5′ terminal nucleotides, e.g., between one and about 5 nucleotides at the 5′ terminus of the antisense strand. As used herein, the “3′ end,” as in the 3′ end of a sense strand, refers to the region, e.g., a region of between one and about 5 nucleotides, that is complementary to the nucleotides of the 5′ end of the complementary antisense strand.

As used herein, the term “capping group” refers to a chemical moiety that replaces a hydrogen atom in a functional group such as an alcohol (ROH), a carboxylic acid (RCO₂H), or an amine (RNH₂). Non-limiting examples of capping groups include alkyl (e.g., methyl, tertiary-butyl); alkenyl (e.g., vinyl, allyl); carboxyl (e.g., acetyl, benzoyl); carbamoyl; phosphate; and phosphonate (e.g., vinylphosphonate). Other suitable capping groups are known to those of skill in the art.

As used herein, the term “base pair” refers to the interaction between pairs of nucleotides (or nucleotide analogs) on opposing strands of an oligonucleotide duplex (e.g., a duplex formed by a strand of a double-stranded oligonucleotide and a target mRNA sequence), due primarily to H-bonding, van der Waals interactions, and the like between said nucleotides (or nucleotide analogs). As used herein, the term “bond strength” or “base pair strength” refers to the strength of the base pair.

“Treatment,” or “treating,” as used herein, refers to the application or administration of a therapeutic agent (e.g., an oligonucleotide conjugate or composition thereof) to a patient, or application or administration of a therapeutic agent to an isolated tissue or cell line from a patient, who has the disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

As used herein the phrase “pharmaceutically acceptable carrier” is intended to include any and all solvents, dispersion media, coatings, antibacterial and antifungal agents, isotonic and absorption delaying agents, and the like, compatible with pharmaceutical administration.

As used herein, “reproductive tissue” refers to male or female reproductive tissue and includes external and internal tissues, such as tissues of the penis, scrotum, testicles, vas deferens, prostate, urethra, seminiferous tubules, epididymis, seminal vesicle ducts, ejaculatory ducts, bulbourethral glands, uterus, ovaries, fallopian tubes, cervix and vagina.

As used herein, “extra-embryonic tissue” refers to a tissue derived from the fertilized egg, that enclose or otherwise contribute to the support of the developing embryo, and which are not retained by after parturition, such as the placenta, extraembryonic membranes, and umbilical cord.

### Conjugated Oligonucleotides

In a first aspect, the present disclosure provides conjugated oligonucleotides represented by formula (I):

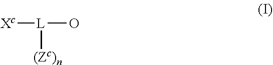

wherein O represents one or more oligonucleotides; L represents a linker; X^{c} represents a hydrophobic moiety; and Z^{c} represents a headgroup.

In another aspect, the present disclosure provides multivalent conjugated oligonucleotides represented by formula (I_{A}):

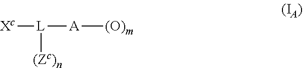

wherein O represents an oligonucleotide; m is at least two; L represents a linker; A represents one or more branching points, one or more spacers, or a combination thereof; X^{c} represents a hydrophobic moiety; and Z^{c} represents a headgroup.

### Variable Z^{c}

In certain embodiments, a conjugated oligonucleotide of formula (I) or multivalent conjugated oligonucleotide of formula (I_{A}) includes a headgroup (Z^{c}) having the structure of formula (II):

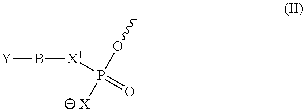

wherein Y includes a hydrophilic moiety, bridge group (B) can be a hydrocarbon chain, optionally having one or more carbon atoms replaced by a heteroatom, and optionally including one or more substituents; X¹ can be absent, an oxygen atom, or NH; X can be oxygen, sulfur or borane; and n can be 1 or 2. In certain embodiments, when n is 1, headgroup (Z^{c}) is not phosphocholine, phosphoserine, or phosphoethanolamine.

In certain embodiments, Y includes —[NR₃]⁺, —[SR₂]⁺, —NR₂, and —CH(NH₂)—CO₂H. At each occurrence, R can be, independently, selected from the group consisting of hydrogen, and optionally substituted C1-C9 alkyl, C1-C9 heteroalkyl, C1-C9 alkenyl, C1-C9 heteroalkenyl, C1-C1 alkynyl, and C1-C9 heteroalkynyl groups.

In certain embodiments, Y can be represented by formula (III):

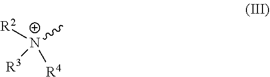

wherein R², R³, and R⁴ can be, independently, selected from a hydrogen atom and substituted or unsubstituted C1-C10 alkyl, alkenyl, alkynyl, heteroalkyl, heteroalkenyl, or heteroalkynyl groups. Heteroalkyl, heteroalkenyl, or heteroalkynyl can include more than one heteroatom. At each occurrence, a heteroatom can be an oxygen, nitrogen, silicon, sulfur, or halogen atom. In certain embodiments, substituents for R², R³, and R⁴ can be, independently, selected from the group consisting of alkyl groups, substituted alkyl, alkenyl, substituted alkenyl, halides, —OR′, —NR′R″, —CF₃, —CN, —NO₂, —C₂R′, —SR′, —N₃, —C(═O)NR′R″, —NR′C(O)R″, —C(═O)R′—C(═O)OR′—OC(O)R′, —O(CR′R″)ᵣC(═O)R′, —O(CR′R″)ᵣNR″C(O)R′, —O(CR′R′)ᵣNR″SO₂R′, —OC(O)NR′R′, —NR′C(O)OR″, —SO₂R′, —SO₂NR′R″, and —NR′SO₂R″. R′ and R″ individually can be hydrogen, C1-C9 alkyl groups, cycloalkyl groups, heterocyclyl groups, aryl groups, or arylalkyl groups, and r is an integer from 1 to 6. In some embodiments, R², R³, and R⁴, independently, can be a hydrogen atom or an unsubstituted C1-C9 alkyl group, such as a methyl, ethyl, propyl, butyl (e.g., t-butyl) groups, or the like.

In one embodiment, B can be any bridge group connecting the phosphorus-containing functional group and variable Y of head group Z^{c}. In certain embodiments, B can be a substituted or unsubstituted C2-C9 alkyl, C2-C9 heteroalkyl, C2-C9 alkenyl, C2-C9 heteroalkenyl, C2-C9 alkynyl, or C2-C9 heteroalkynyl group. In certain embodiments, B includes one or more substituents, independently, selected from the group consisting of alkyl, substituted alkyl, alkenyl, substituted alkenyl, halides, —OR′, —NR′R″, —CF3, —CN, —NO2, —C2R′, —SR′, —N3, —C(═O)NR′R″, —NR′C(O)R″, —C(═O)R′—C(═O)OR′—OC(O)R′, —OC(O)NR′R″, —NR′C(O)OR″, —SO2R′, —SO2NR′R″, and —NR′SO2R″, wherein R′ and R″ are individually hydrogen or C1-C10 alkyl groups.

In certain embodiments, a conjugated oligonucleotide of formula (I) or multivalent conjugated oligonucleotide of formula (I_{A}) includes one of the following headgroups (Z^{c}):

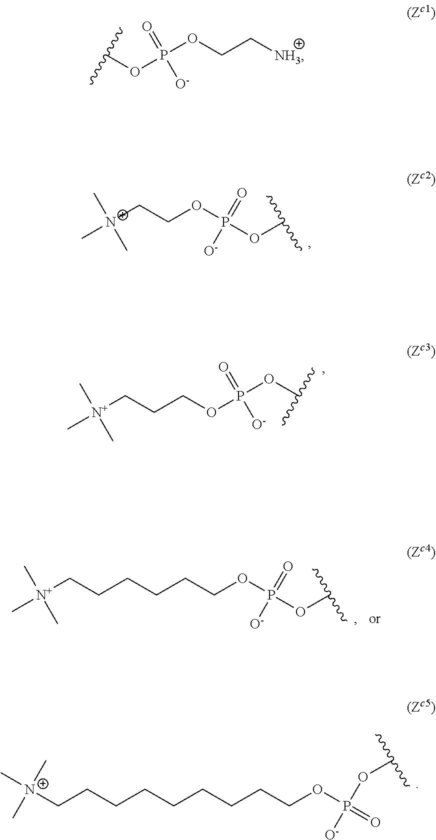

For example, a conjugated oligonucleotide for targeting extrahepatic tissues such as spleen, adrenal gland, pancreas, kidney, lung, heart, kidney, and urinary bladder can include a phosphoethanolamine (Z^{c1}) headgroup. In some embodiments, a hydrophobically conjugated oligonucleotide comprising one or more Z^{c2}, Z^{c3}, Z^{c4}, or Z^{c5} headgroups can be used to improve accumulation and/or efficacy of an oligonucleotide in liver, heart, muscle, and/or lung tissue as compared with a corresponding hydrophobically conjugated oligonucleotide lacking a headgroup or having a single phosphocholine headgroup.

### Variable L

In one embodiment, L comprises an optionally substituted hydrocarbon chain, optionally including one or more heteroatoms. For example, L can be an ethylene glycol chain, an alkyl chain, a peptide, RNA, DNA, a phosphodiester, a phosphorothioate, a phosphoramidate, an amide, a carbamate, or a combination thereof. L can be trivalent or tetravalent. In an embodiment, the trivalent or tetravalent linker is selected from the group consisting of:

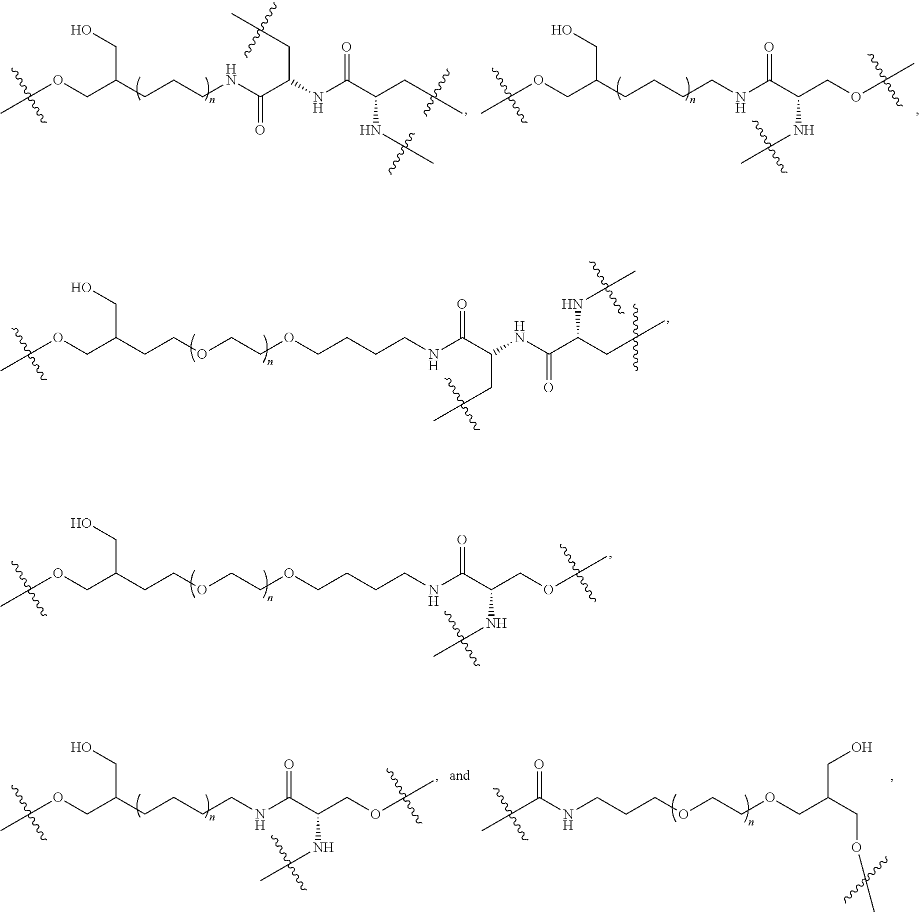

wherein n is 0, 1, 2, 3, 4, or 5.

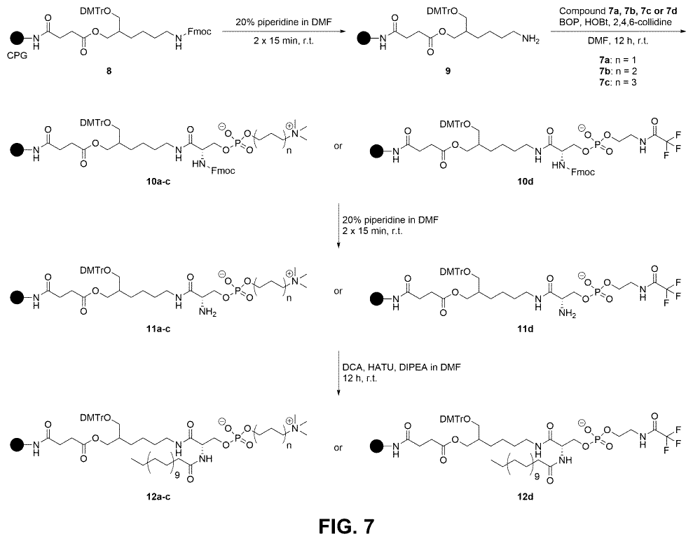

In a particular embodiment, L is the trivalent linker L1 (also referred to herein as C7 (FIG. 7)):

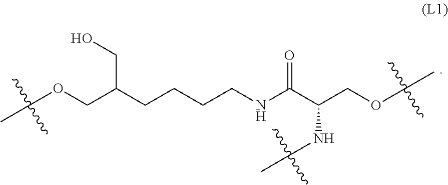 [SMILES: *N[C@@H](CO*)C(=O)NCCCCC(CO)CO*]

In another particular embodiment, L is the tetravalent linker L2:

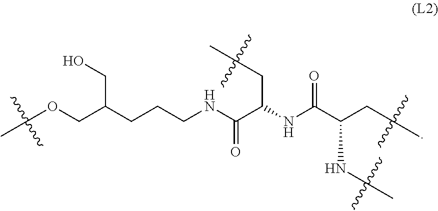

In certain embodiments, L is connected to O, or X^{c}, or Z^{c} via one or more branching points and/or spacers (A). A branching (or brach) point can be any polyvalent organic species or derivative thereof. For example, a branching point can be selected from the group consisting of triols, tetrols, tri-carboxylic acids, tetra-carboxylic acids, tertiary amines, triamines, tetramines, and amino acids. In some cases, L connects three or more compounds via 1 to 3 branching points. A spacer can have the same or similar structure as L. For example, S can be an ethylene glycol chain, an alkyl chain, a peptide, RNA, DNA, a phosphate, a phosphonate, a phosphoramidate, an ester, an amide, a triazole, or include a combination thereof.

### Variable X^{c}

A hydrophobic moiety X^{c} can include any moiety that can be chemically conjugated to an oligonucleotide: to enhance the hydrophobic character of the conjugate. In one embodiment, X^{c} has an affinity for low density lipoprotein and/or intermediate density lipoprotein. In a related embodiment, X^{c} is a saturated or unsaturated moiety having fewer than three double bonds. In another embodiment, X^{c} has an affinity for high density lipoprotein. In a related embodiment, X^{c} is a polyunsaturated moiety having at three or more double bonds (e.g., having three, four, five, six, seven, eight, nine or ten double bonds). In a particular embodiment, X^{c} is a polyunsaturated moiety having three double bonds. In a particular embodiment, X^{c} is a polyunsaturated moiety having four double bonds. In a particular embodiment, X^{c} is a polyunsaturated moiety having five double bonds. In a particular embodiment, X^{c} is a polyunsaturated moiety having six double bonds. In another embodiment, X^{c} is selected from the group consisting of fatty acids, steroids, secosteroids, lipids, gangliosides and nucleoside analogs, and endocannabinoids.

In another embodiment, X^{c} is a neuromodulatory lipid, e.g., an endocannabinoid. Non-limiting examples of endocannabinoids include Anandamide, Arachidonoylethanolamine, 2-Arachidonyl glyceryl ether (noladin ether), 2-Arachidonyl glyceryl ether (noladin ether), 2-Arachidonoylglycerol, and N-Arachidonoyl dopamine.

In another embodiment, X^{c} is an omega-3 fatty acid. Non-limiting examples of omega-3 fatty acids include Hexadecatrienoic acid (HTA), Alpha-linolenic acid (ALA), Stearidonic acid (SDA), Eicosatrienoic acid (ETE), Eicosatetraenoic acid (ETA), Eicosapentaenoic acid (EPA, Timnodonic acid), Heneicosapentaenoic acid (HPA), Docosapentaenoic acid (DPA, Clupanodonic acid), Docosahexaenoic acid (DHA, Cervonic acid), Tetracosapentaenoic acid, and Tetracosahexaenoic acid (Nisinic acid).

In another embodiment, X^{c} is an omega-6 fatty acid. Non-limiting examples of omega-6 fatty acids include Linoleic acid, Gamma-linolenic acid (GLA), Eicosadienoic acid, Dihomo-gamma-linolenic acid (DGLA), Arachidonic acid (AA), Docosadienoic acid, Adrenic acid, Docosapentaenoic acid (Osbond acid), Tetracosatetraenoic acid, and Tetracosapentaenoic acid.

In another embodiment, X^{c} is an omega-9 fatty acid. Non-limiting examples of omega-9 fatty acids include Oleic acid, Eicosenoic acid, Mead acid, Erucic acid, and Nervonic acid.

In another embodiment, X^{c} is a conjugated linolenic acid. Non-limiting examples of conjugated linolenic acids include α-Calendic acid, β-Calendic acid, Jacaric acid, α-Eleostearic acid, β-Eleostearic acid, Catalpic acid, and Punicic acid.

In another embodiment, X^{c} is a saturated fatty acid. Non-limiting examples of saturated fatty acids include Caprylic acid, Capric acid, Docosanoic acid (DCA), Lauric acid, Myristic acid, Palmitic acid, Stearic acid, Arachidic acid, Behenic acid, Lignoceric acid, and Cerotic acid.

In another embodiment, X^{c} is an unsaturated fatty acid. Non-limiting examples unsaturated fatty acids include Rumelenic acid, α-Parinaric acid, β-Parinaric acid, Bosseopentaenoic acid, Pinolenic acid, and Podocarpic acid.

In another embodiment, X^{c} is selected from the group consisting of docosanoic acid (DCA), docosahexaenoic acid (DHA), and eicosapentaenoic acid (EPA). In a particular embodiment, X^{c} is docosanoic acid (DCA). In another particular embodiment, X^{c} is DHA. In another particular embodiment, X^{c} is EPA.

In another embodiment, X^{c} is a secosteroid. In a particular embodiment, X^{c} is calciferol, Vitamin D, Vitamin D₂, or Vitamin D₃.

In another embodiment, X^{c} is a steroid. In a particular embodiment, X^{c} is a steroid other than cholesterol.

In a particular embodiment, X^{c} is not cholesterol.

In another embodiment, X^{c} is an alkyl chain, a vitamin, a peptide, or a bioactive conjugate (including but not limited to glycosphingolipids, polyunsaturated fatty acids, secosteroids, steroid hormones, sterol lipids).

In another embodiment of the oligonucleotide, X^{c} can be characterized by a clogP value in a range selected from: −10 to −9, −9 to −8, −8 to −7, −7 to −6, −6 to −5, −5 to −4, −4 to −3, −3 to −2, −2 to −1, −1 to 0, 0 to 1, 1 to 2, 2 to 3, 3 to 4, 4 to 5, 5 to 6, 6 to 7, 7 to 8, 8 to 9, and 9 to 10.

### Variable O

A conjugated oligonucleotide of formula (I) or (I_{A}) can include DNA, siRNA, shRNA, antagomiR, miRNA, antisense molecules, gapmers, mixmers, and guide RNA. An oligonucleotide can be single-stranded or double-stranded. Each strand can have a 5′ end and a 3′ end. A oligonucleotide can be conjugated via the 5′ end or the 3′ end.

Conjugation can be accomplished by methods known in the art, e.g., using the methods of Lambert et al., Drug Deliv. Rev.: 47(1), 99-112 (2001) (describes nucleic acids loaded to polyalkylcyanoacrylate (PACA) nanoparticles); Fattal et al., J. Control Release 53(1-3):137-43 (1998) (describes nucleic acids bound to nanoparticles); Schwab et al., Ann. Oncol. 5 Suppl. 4:55-8 (1994) (describes nucleic acids linked to intercalating agents, hydrophobic groups, polycations or PACA nanoparticles); and Godard et al., Eur. J. Biochem. 232(2):404-10 (1995) (describes nucleic acids linked to nanoparticles).

In some embodiments, a conjugated oligonucleotide includes an siRNA molecule. An siRNA molecule of the present disclosure is a duplex consisting of a sense strand and complementary antisense strand, the antisense strand having sufficient complementary to a target to mediate RNAi. Preferably, the siRNA molecule has a length from about 10-50 or more nucleotides, i.e., each strand comprises 10-50 nucleotides (or nucleotide analogs). More preferably, the siRNA molecule has a length from about 16-30, e.g., 16, 17, 18, 19, 20, 21, 22, 23, 24, 25, 26, 27, 28, 29, or 30 nucleotides in each strand, wherein one of the strands is sufficiently complementary to a target region. Preferably, the strands are aligned such that there are at least 1, 2, or 3 bases at the end of the strands which do not align (i.e., for which no complementary bases occur in the opposing strand) such that an overhang of 1, 2 or 3 residues occurs at one or both ends of the duplex when strands are annealed. Preferably, the siRNA molecule has a length from about 10-50 or more nucleotides, i.e., each strand comprises 10-50 nucleotides (or nucleotide analogs). More preferably, the siRNA molecule has a length from about 16-30, e.g., 16, 17, 18, 19, 20, 21, 22, 23, 24, 25, 26, 27, 28, 29, or 30 nucleotides in each strand, wherein one of the strands is substantially complementary to a target sequence, and the other strand is identical or substantially identical to the first strand.

Generally, siRNAs can be designed by using any method known in the art, for instance, by using the following protocol:

1. An siRNA should be specific for a target sequence. In one embodiment, the is found in a mutant huntingtin (htt) allele, but not a wild-type huntingtin allele. In another embodiment, a target sequence is found in both a mutant huntingtin (htt) allele, and a wild-type huntingtin allele. In another embodiment, a target sequence is found in a wild-type huntingtin allele. The first strand should be complementary to the target sequence, and the other strand is substantially complementary to the first strand. In one embodiment, the target sequence is outside the expanded CAG repeat of the mutant huntingin (htt) allele. In another embodiment, the target sequence is outside a coding region of the target gene. Exemplary target sequences are selected from the 5′ untranslated region (5′-UTR) or an intronic region of a target gene. Cleavage of mRNA at these sites should eliminate translation of corresponding mutant protein. Target sequences from other regions of the htt gene are also suitable for targeting. A sense strand is designed based on the target sequence. Further, siRNAs with lower G/C content (35-55%) may be more active than those with G/C content higher than 55%. Thus in one embodiment, the present disclosure provides nucleic acid molecules having 35-55% G/C content.

2. The sense strand of the siRNA is designed based on the sequence of the selected target site. Preferably the sense strand includes about 19 to 25 nucleotides, e.g., 19, 20, 21, 22, 23, 24 or 25 nucleotides. More preferably, the sense strand includes 21, 22 or 23 nucleotides. The skilled artisan will appreciate, however, that siRNAs having a length of less than 19 nucleotides or greater than 25 nucleotides can also function to mediate RNAi. Accordingly, siRNAs of such length are also within the scope of the present disclosure provided that they retain the ability to mediate RNAi. Longer oligonucleotides agents have been demonstrated to elicit an interferon or Protein Kinase R (PKR) response in certain mammalian cells which may be undesirable. Preferably the oligonucleotides of the present disclosure do not elicit a PKR response (i.e., are of a sufficiently short length). However, longer oligonucleotides may be useful, for example, in cell types incapable of generating a PRK response or in situations where the PKR response has been down-regulated or dampened by alternative means. The siRNA molecules of the present disclosure have sufficient complementarity with the target sequence such that the siRNA can mediate RNAi. A sense strand of siRNA can be designed have to have a sequence sufficiently identical to a portion of the target. For example, the sense strand may have 100% identity to the target site. However, 100% identity is not required. Greater than 80% identity, e.g., 80%, 81%, 82%, 83%, 84%, 85%, 86%, 87%, 88%, 89%, 90%, 91%, 92%, 93%, 94%, 95%, 96%, 97%, 98%, 99% or even 100% identity, between the sense strand and the target RNA sequence is preferred. The oligonucleotides of the present disclosure have the advantage of being able to tolerate certain sequence variations to enhance efficiency and specificity of RNAi. In one embodiment, the sense strand has 4, 3, 2, 1, or 0 mismatched nucleotide(s) with a target region, such as a target region that differs by at least one base pair between a wild-type and mutant allele, e.g., a target region comprising the gain-of-function mutation, and the other strand is identical or substantially identical to the first strand. Moreover, siRNA sequences with small insertions or deletions of 1 or 2 nucleotides may also be effective for mediating RNAi. Alternatively, siRNA sequences with nucleotide analog substitutions or insertions can be effective for inhibition. In one embodiment, the anti-sense strand is complementary to a sequence having 4, 3, 2, 1, or 0 mismatched nucleotide(s) with a target region, such as a target region that differs by at least one base pair between a wild-type and mutant allele, e.g., a target region comprising the gain-of-function mutation, and the sense strand is identical or substantially identical to the antisense strand. Sequence identity may be determined by sequence comparison and alignment algorithms known in the art. To determine the percent identity of two nucleic acid sequences (or of two amino acid sequences), the sequences are aligned for optimal comparison purposes (e.g., gaps can be introduced in the first sequence or second sequence for optimal alignment). The nucleotides (or amino acid residues) at corresponding nucleotide (or amino acid) positions are then compared. When a position in the first sequence is occupied by the same residue as the corresponding position in the second sequence, then the molecules are identical at that position. The percent identity between the two sequences is a function of the number of identical positions shared by the sequences (i.e., percent (%) homology=number of identical positions/total number of positions×100), optionally penalizing the score for the number of gaps introduced and/or length of gaps introduced.

The comparison of sequences and determination of percent identity between two sequences can be accomplished using a mathematical algorithm. In one embodiment, the alignment generated over a certain portion of the sequence aligned having sufficient identity but not over portions having low degree of identity (i.e., a local alignment). A non-limiting example of a local alignment algorithm utilized for the comparison of sequences is the algorithm of Karlin and Altschul (1990) Proc. Natl. Acad. Sci. USA 87:2264-68, modified as in Karlin and Altschul (1993) Proc. Natl. Acad. Sci. USA 90:5873-77. Such an algorithm is incorporated into the BLAST programs (version 2.0) of Altschul, et al. (1990) J. Mol. Biol. 215:403-10. In another embodiment, the alignment is optimized by introducing appropriate gaps and percent identity is determined over the length of the aligned sequences (i.e., a gapped alignment). To obtain gapped alignments for comparison purposes, Gapped BLAST can be utilized as described in Altschul et al., (1997) Nucleic Acids Res. 25(17):3389-3402. In another embodiment, the alignment is optimized by introducing appropriate gaps and percent identity is determined over the entire length of the sequences aligned (i.e., a global alignment). A non-limiting example of a mathematical algorithm utilized for the global comparison of sequences is the algorithm of Myers and Miller, CABIOS (1989). Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

3. The antisense or guide strand of the siRNA is routinely the same length as the sense strand and includes complementary nucleotides. In one embodiment, the guide and sense strands are fully complementary, i.e., the strands are blunt-ended when aligned or annealed. In another embodiment, the strands of the siRNA can be paired in such a way as to have a 3′ overhang of 1 to 4, e.g., 2, nucleotides. Overhangs can comprise (or consist of) nucleotides corresponding to the target gene sequence (or complement thereof). Alternatively, overhangs can comprise (or consist of) deoxyribonucleotides, for example dTs, or nucleotide analogs, or other suitable non-nucleotide material. Thus in another embodiment, the nucleic acid molecules may have a 3′ overhang of 2 nucleotides, such as TT. The overhanging nucleotides may be either RNA or DNA. As noted above, it is desirable to choose a target region wherein the mutant:wild type mismatch is a purine:purine mismatch.

4. Using any method known in the art, compare the potential targets to the appropriate genome database (human, mouse, rat, etc.) and eliminate from consideration any target sequences with significant homology to other coding sequences. One such method for such sequence homology searches is known as BLAST, which is available at National Center for Biotechnology Information website.

5. Select one or more sequences that meet the predetermined criteria for evaluation. Further general information about the design and use of siRNA may be found in “The siRNA User Guide,” available at The Max-Plank-Institut fur Biophysikalishe Chemie website.

Alternatively, the siRNA may be defined functionally as a nucleotide sequence (or oligonucleotide sequence) that is capable of hybridizing with the target sequence (e.g., 400 mM NaCl, 40 mM PIPES pH 6.4, 1 mM EDTA, 50° C. or 70° C. hybridization for 12-16 hours; followed by washing). Additional hybridization conditions can include hybridization at 70° C. in 1×SSC or 50° C. in 1×SSC, 50% formamide followed by washing at 70° C. in 0.3×SSC or hybridization at 70° C. in 4×SSC or 50° C. in 4×SSC, 50% formamide followed by washing at 67° C. in 1×SSC. The hybridization temperature for hybrids anticipated to be less than 50 base pairs in length should be 5-10° C. less than the melting temperature (Tm) of the hybrid, where Tm is determined according to the following equations. For hybrids less than 18 base pairs in length, Tm(° C.)=2(# of A+T bases)+4(# of G+C bases). For hybrids between 18 and 49 base pairs in length, Tm(° C.)=81.5+16.6(log 10[Na+])+0.41(% G+C)−(600/N), where N is the number of bases in the hybrid, and [Na+] is the concentration of sodium ions in the hybridization buffer ([Na+] for 1×SSC=0.165 M). Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, N.Y., chapters 9 and 11, and Current Protocols in Molecular Biology, 1995, F. M. Ausubel et al., eds., John Wiley & Sons, Inc., sections 2.10 and 6.3-6.4, incorporated herein by reference.

Negative control siRNAs should have the same nucleotide composition as the selected siRNA, but without significant sequence complementarity to the appropriate genome. Such negative controls may be designed by randomly scrambling the nucleotide sequence of the selected siRNA. A homology search can be performed to ensure that the negative control lacks homology to any other gene in the appropriate genome. In addition, negative control siRNAs can be designed by introducing one or more base mismatches into the sequence.

6. To validate the effectiveness by which siRNAs destroy target mRNAs (e.g., wild-type or mutant huntingtin mRNA), the siRNA may be incubated with target cDNA (e.g., huntingtin cDNA) in a Drosophila-based in vitro mRNA expression system. Radiolabeled with ³²P, newly synthesized target mRNAs (e.g., huntingtin mRNA) are detected autoradiographically on an agarose gel. The presence of cleaved target mRNA indicates mRNA nuclease activity. Suitable controls include omission of siRNA and use of non-target cDNA. Alternatively, control siRNAs are selected having the same nucleotide composition as the selected siRNA, but without significant sequence complementarity to the appropriate target gene. Such negative controls can be designed by randomly scrambling the nucleotide sequence of the selected siRNA. A homology search can be performed to ensure that the negative control lacks homology to any other gene in the appropriate genome. In addition, negative control siRNAs can be designed by introducing one or more base mismatches into the sequence.

siRNAs may be designed to interact with a target sequence. Said siRNAs comprise an antisense strand which is sufficiently complementary with the target sequence to mediate silencing of the target sequence. In certain embodiments, the oligonucleotide is a siRNA. Sites of siRNA-mRNA complementation are selected which result in optimal mRNA specificity and maximal mRNA cleavage.

siRNA-Like Molecules

siRNA-like molecules of the present disclosure have a sequence (i.e., have a strand having a sequence) that is “substantially complementary” to a target sequence of an mRNA (e.g., htt mRNA). A substantially complementary sequence is sufficiently complementary to direct gene silencing either by RNAi or translational repression. A siRNA-like molecule is designed in the same way as siRNA molecules, but the degree of sequence identity between the sense strand and target RNA approximates that observed between an miRNA and its target. In general, as the degree of sequence identity between a miRNA sequence and the corresponding target gene sequence is decreased, the tendency to mediate post-transcriptional gene silencing by translational repression rather than RNAi is increased. Therefore, in an alternative embodiment, where post-transcriptional gene silencing by translational repression of the target gene is desired, the miRNA sequence has partial complementarity with the target gene sequence. In certain embodiments, the miRNA sequence has partial complementarity with one or more short sequences (complementarity sites) dispersed within the target mRNA (e.g., within the 3′-UTR of the target mRNA) (Hutvagner and Zamore, Science, 2002; Zeng et al., Mol. Cell, 2002; Zeng et al., RNA, 2003; Doench et al., Genes & Dev., 2003). Since the mechanism of translational repression is cooperative, multiple complementarity sites (e.g., 2, 3, 4, 5, or 6) may be targeted in certain embodiments.

The capacity of a siRNA-like duplex to mediate RNAi or translational repression may be predicted by the distribution of non-identical nucleotides between the target gene sequence and the nucleotide sequence of the silencing agent at the site of complementarity. In one embodiment, where gene silencing by translational repression is desired, at least one non-identical nucleotide is present in the central portion of the complementarity site so that duplex formed by the miRNA guide strand and the target mRNA contains a central “bulge” (Doench J G et al., Genes & Dev., 2003). In another embodiment 2, 3, 4, 5, or 6 contiguous or non-contiguous non-identical nucleotides are introduced. The non-identical nucleotide may be selected such that it forms a wobble base pair (e.g., G:U) or a mismatched base pair (G:A, C:A, C:U, G:G, A:A, C:C, U:U). In a further embodiment, the “bulge” is centered at nucleotide positions 12 and 13 from the 5′ end of the miRNA molecule.

In one embodiment, O comprises one or more double stranded (ds) RNA compounds. The dsRNA can include a sense strand of at least 15 contiguous nucleotides, said sense strand having a 5′ end, a 3′ end, and homology with a target. The sense strand can be conjugated at the 3′ end to -L described above. The dsRNA can include an antisense strand of at least 16 contiguous nucleotides, said antisense strand having a 5′ end, a 3′ end and complementarity to a target. In one embodiment, the antisense strand has sufficient complementarity to the target to hybridize. In certain embodiments, the complementarity is >95%, >90%, >85%, >80%, >75%, >70%, >65%, >60%, >55% or >50%. In one embodiment, an antisense strand of variable O has perfect complementarity to the target. In one embodiment, the sense strand has complete homology with the target. In a particular embodiment, the target is mammalian or viral mRNA. In another particular embodiment, the target is an intronic region of said mRNA.

### Modified Oligonucleotides

In certain aspects of the present disclosure, an oligonucleotide (or any portion thereof) of the present disclosure as described above may be modified such that the activity is further improved. For example, an oligonucleotide, such as a dsRNA or siRNA described above may be modified with any of the modifications 1-5 described below. The modifications can, in part, serve to further enhance target discrimination, to enhance stability of the agent (e.g., to prevent degradation), to promote cellular uptake, to enhance the target efficiency, to improve efficacy in binding (e.g., to the targets), to improve patient tolerance to the agent, and/or to reduce toxicity.

In certain embodiments, a dsRNA of the present disclosure may be substituted with a destabilizing nucleotide to enhance single nucleotide target discrimination (see U.S. application Ser. No. 11/698,689, filed Jan. 25, 2007, and U.S. Provisional Application No. 60/762,225 filed Jan. 25, 2006, both of which are incorporated herein by reference). Such a modification may be sufficient to abolish the specificity of a dsRNA for a non-target mRNA (e.g. wild-type mRNA), without appreciably affecting the specificity of a dsRNA for a target mRNA (e.g. gain-of-function mutant mRNA).

A dsRNA of the present disclosure can be modified by the introduction of at least one universal nucleotide in the antisense strand thereof. Universal nucleotides comprise base portions that are capable of base pairing indiscriminately with any of the four conventional nucleotide bases (e.g. A, G, C, U). A universal nucleotide will have a relatively minor effect on the stability of the RNA duplex or the duplex formed by the guide strand of the dsRNA and the target mRNA. Exemplary universal nucleotide include those having an inosine base portion or an inosine analog base portion selected from the group consisting of deoxyinosine (e.g. 2′-deoxyinosine), 7-deaza-2′-deoxyinosine, 2′-aza-2′-deoxyinosine, PNA-inosine, morpholino-inosine, LNA-inosine, phosphoramidate-inosine, 2′-O-methoxyethyl-inosine, and 2′-OMe-inosine. In certain embodiments, the universal nucleotide is an inosine residue or a naturally occurring analog thereof.

An oligonucleotide of the present disclosure can be modified by the introduction of at least one destabilizing nucleotide within 5 nucleotides from a specificity-determining nucleotide (i.e., the nucleotide which recognizes the disease-related polymorphism). For example, the destabilizing nucleotide may be introduced at a position that is within 5, 4, 3, 2, or 1 nucleotide(s) from a specificity-determining nucleotide. In exemplary embodiments, the destabilizing nucleotide is introduced at a position which is 3 nucleotides from the specificity-determining nucleotide (i.e., such that there are 2 stabilizing nucleotides between the destabilizing nucleotide and the specificity-determining nucleotide). In an oligonucleotides having two strands or strand portions (e.g. siRNAs and shRNAs), the destabilizing nucleotide may be introduced in the strand or strand portion that does not contain the specificity-determining nucleotide. The destabilizing nucleotide can be introduced in the same strand or strand portion that contains the specificity-determining nucleotide.

In certain embodiments, a dsRNA of the present disclosure may be altered to facilitate enhanced efficacy and specificity in mediating RNAi according to asymmetry design rules (see U.S. Pat. Nos. 8,309,704, 7,750,144, 8,304,530, 8,329,892 and 8,309,705). Such alterations facilitate entry of the antisense strand of an siRNA (e.g., an siRNA designed using the methods of the present disclosure or an siRNA produced from a shRNA) into RISC in favor of the sense strand, such that the antisense strand preferentially guides cleavage or translational repression of a target mRNA, and thus increasing or improving the efficiency of target cleavage and silencing. Preferably the asymmetry of a dsRNA is enhanced by lessening the base pair strength between the antisense strand 5′ end (AS 5′) and the sense strand 3′ end (S 3′) of a dsRNA relative to the bond strength or base pair strength between the antisense strand 3′ end (AS 3′) and the sense strand 5′ end (S '5) of the dsRNA.

In one embodiment, the asymmetry of a dsRNA of the present disclosure may be enhanced such that there are fewer G:C base pairs between the 5′ end of the first or antisense strand and the 3′ end of the sense strand portion than between the 3′ end of the first or antisense strand and the 5′ end of the sense strand portion. In another embodiment, the asymmetry of a dsRNA of the present disclosure may be enhanced such that there is at least one mismatched base pair between the 5′ end of the first or antisense strand and the 3′ end of the sense strand portion. Preferably, the mismatched base pair is selected from the group consisting of G:A, C:A, C:U, G:G, A:A, C:C and U:U. In another embodiment, the asymmetry of a dsRNA of the present disclosure may be enhanced such that there is at least one wobble base pair, e.g., G:U, between the 5′ end of the first or antisense strand and the 3′ end of the sense strand portion. In another embodiment, the asymmetry of a dsRNA of the present disclosure may be enhanced such that there is at least one base pair comprising a rare nucleotide, e.g., inosine (I). Preferably, the base pair is selected from the group consisting of an I:A, I:U and I:C. In yet another embodiment, the asymmetry of a dsRNA of the present disclosure may be enhanced such that there is at least one base pair comprising a modified nucleotide. The modified nucleotide can be selected from the group consisting of 2-amino-G, 2-amino-A, 2,6-diamino-G, and 2,6-diamino-A.

An oligonucleotide of the present disclosure can be modified to improve stability in serum or in growth medium for cell cultures. In order to enhance the stability, the 3′-residues may be stabilized against degradation, e.g., they may be selected such that they consist of purine nucleotides, particularly adenosine or guanosine nucleotides. Alternatively, substitution of pyrimidine nucleotides by modified analogues, e.g., substitution of uridine by 2′-deoxythymidine is tolerated and does not affect the efficiency of RNA interference.

A dsRNA molecules that includes first and second strands wherein the second strand and/or first strand can be modified by the substitution of internal nucleotides with modified nucleotides, such that in vivo stability is enhanced as compared to a corresponding unmodified oligonucleotide. As defined herein, an “internal” nucleotide is one occurring at any position other than the 5′ end or 3′ end of nucleic acid molecule, polynucleotide or oligonucleotide. An internal nucleotide can be within a single-stranded molecule or within a strand of a duplex or double-stranded molecule. In one embodiment, the sense strand and/or antisense strand is modified by the substitution of at least one internal nucleotide. In another embodiment, the sense strand and/or antisense strand is modified by the substitution of at least 2, 3, 4, 5, 6, 7, 8, 9, 10, 11, 12, 13, 14, 15, 16, 17, 18, 19, 20, 21, 22, 23, 24, 25 or more internal nucleotides. In another embodiment, the sense strand and/or antisense strand is modified by the substitution of at least 5%, 10%, 15%, 20%, 25%, 30%, 35%, 40%, 45%, 50%, 55%, 60%, 65%, 70%, 75%, 80%, 85%, 90%, 95% or more of the internal nucleotides. In yet another embodiment, the sense strand and/or antisense strand is modified by the substitution of all of the internal nucleotides.

An oligonucleotide may contain at least one modified nucleotide analogue. The nucleotide analogues may be located at positions where the target-specific silencing activity, e.g., the RNAi mediating activity or translational repression activity is not substantially effected, e.g., in a region at the 5′-end and/or the 3′-end of the siRNA molecule. The ends may be stabilized by incorporating modified nucleotide analogues.

Exemplary nucleotide analogues include sugar- and/or backbone-modified ribonucleotides (i.e., include modifications to the phosphate-sugar backbone). For example, the phosphodiester linkages of natural RNA may be modified to include at least one of a nitrogen or sulfur heteroatom. In exemplary backbone-modified ribonucleotides, the phosphoester group connecting to adjacent ribonucleotides is replaced by a modified group, e.g., of phosphothioate group. In exemplary sugar-modified ribonucleotides, the 2′ OH-group is replaced by a group selected from H, OR, R, halo, SH, SR, NH₂, NHR, NR₂ or ON, wherein R is C₁-C₆ alkyl, alkenyl or alkynyl and halo is F, Cl, Br or I.

In particular embodiments, the modifications are 2′-fluoro, 2′-amino and/or 2′-thio modifications. Non-limiting modifications include 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine, 2,6-diaminopurine, 4-thio-uridine, and/or 5-amino-allyl-uridine. In a particular embodiment, the 2′-fluoro ribonucleotides are every uridine and cytidine. Additional exemplary modifications include 5-bromo-uridine, 5-iodo-uridine, 5-methyl-cytidine, ribo-thymidine, 2-aminopurine, 2′-amino-butyryl-pyrene-uridine, 5-fluoro-cytidine, and 5-fluoro-uridine. 2′-deoxy-nucleotides and 2′-Ome nucleotides can also be used within modified RNA-silencing agents moieties of the present disclosure. Additional modified residues include, deoxy-abasic, inosine, N3-methyl-uridine, N6,N6-dimethyl-adenosine, pseudouridine, purine ribonucleoside and ribavirin. In certain embodiments, the 2′ moiety is a methyl group such that the linking moiety is a 2′-O-methyl oligonucleotide.

An oligonucleotide of the present disclosure can comprise Locked Nucleic Acids (LNAs). LNAs comprise sugar-modified nucleotides that resist nuclease activities (are highly stable) and possess single nucleotide discrimination for mRNA (Elmen et al., Nucleic Acids Res., (2005), 33(1): 439-447; Braasch et al. (2003) Biochemistry 42:7967-7975, Petersen et al. (2003) Trends Biotechnol 21:74-81). These molecules have 2′-O,4′-C-ethylene-bridged nucleic acids, with possible modifications such as 2′-deoxy-2″-fluorouridine. Moreover, LNAs increase the specificity of oligonucleotides by constraining the sugar moiety into the 3′-endo conformation, thereby pre-organizing the nucleotide for base pairing and increasing the melting temperature of the oligonucleotide by as much as 10° C. per base.

An oligonucleotide of the present disclosure can comprise Peptide Nucleic Acids (PNAs). PNAs comprise modified nucleotides in which the sugar-phosphate portion of the nucleotide is replaced with a neutral 2-amino ethylglycine moiety capable of forming a polyamide backbone which is highly resistant to nuclease digestion and imparts improved binding specificity to the molecule (Nielsen, et al., Science, (2001), 254: 1497-1500).

An oligonucleotide of the present disclosure can comprise nucleobase-modified ribonucleotides, i.e., ribonucleotides, containing at least one non-naturally occurring nucleobase instead of a naturally occurring nucleobase. Bases may be modified to block the activity of adenosine deaminase. Exemplary modified nucleobases include, but are not limited to, uridine and/or cytidine modified at the 5-position, e.g., 5-(2-amino)propyl uridine, 5-bromo uridine; adenosine and/or guanosines modified at the 8 position, e.g., 8-bromo guanosine; deaza nucleotides, e.g., 7-deaza-adenosine; 0- and N-alkylated nucleotides, e.g., N6-methyl adenosine are suitable. Above modifications can be combined in an oligonucleotide.

Oligonucleotide ends can be stabilized by incorporating a modified nucleotide analogue as a capping group. In certain embodiments, an oligonucleotide can be modified with a capping group selected from:

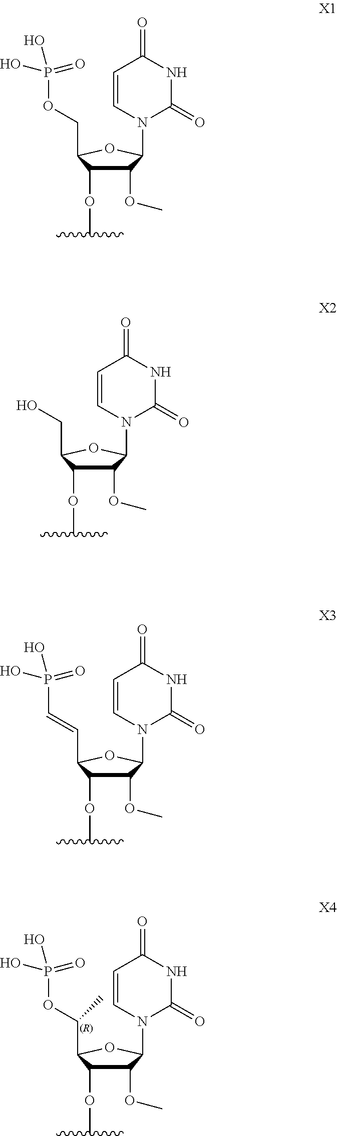

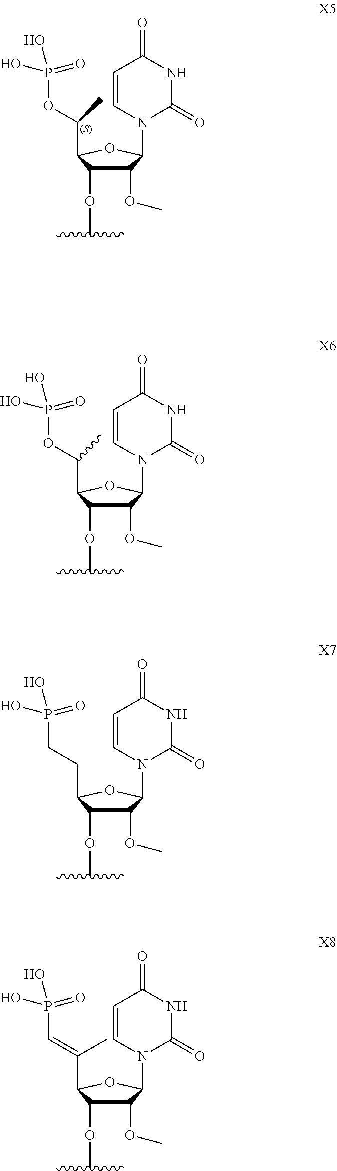

Other exemplary modifications include: (a) 2′ modification, e.g., provision of a 2′ OMe moiety on a U in a sense or antisense strand, but especially on a sense strand, or provision of a 2′ OMe moiety in a 3′ overhang, e.g., at the 3′ terminus (3′ terminus means at the 3′ atom of the molecule or at the most 3′ moiety, e.g., the most 3′ P or 2′ position, as indicated by the context); (b) modification of the backbone, e.g., with the replacement of an 0 with an S, in the phosphate backbone, e.g., the provision of a phosphorothioate modification, on the U or the A or both, especially on an antisense strand; e.g., with the replacement of a P with an S; (c) replacement of the U with a C5 amino linker; (d) replacement of an A with a G (sequence changes are preferred to be located on the sense strand and not the antisense strand); and (d) modification at the 2′, 6′, 7′, or 8′ position. Exemplary embodiments are those in which one or more of these modifications are present on the sense but not the antisense strand, or embodiments where the antisense strand has fewer of such modifications. Yet other exemplary modifications include the use of a methylated P in a 3′ overhang, e.g., at the 3′ terminus; combination of a 2′ modification, e.g., provision of a 2′ O Me moiety and modification of the backbone, e.g., with the replacement of a P with an S, e.g., the provision of a phosphorothioate modification, or the use of a methylated P, in a 3′ overhang, e.g., at the 3′ terminus; modification with a 3′ alkyl; modification with an abasic pyrrolidone in a 3′ overhang, e.g., at the 3′ terminus; modification with naproxen, ibuprofen, or other moieties which inhibit degradation at the 3′ terminus.

In one embodiment, the one or more dsRNA compounds comprises one or more chemically-modified nucleotides. In a particular embodiment, the at least one strand of the dsRNA comprises alternating 2′-methoxy-nucleotides and 2′-fluoro-nucleotides. In another particular embodiment, the nucleotides at positions 1 and 2 from the 3′ end of at least one strand of the dsRNA are connected to adjacent nucleotides via phosphorothioate linkages. In yet another particular embodiment, the nucleotides at positions 1 and 2 from the 3′ end of the at least one strand of the dsRNA and the nucleotides at positions 1 and 2 from the 5′ end of the at least one strand of the dsRNA are connected to adjacent nucleotides via phosphorothioate linkages.

In one embodiment, an antisense strand of variable O includes at least 16 contiguous nucleotides, a 5′ end, a 3′ end, has complementarity to a target, and comprises alternating 2′-methoxy-nucleotides and 2′-fluoro-nucleotides. Further, in some cases, the nucleotides at positions 2 and 14 from the 5′ end of the antisense strand are not 2′-methoxy-nucleotides; the nucleotides are connected via phosphodiester or phosphorothioate linkages; and/or the nucleotides at positions 1-6 from the 3′ end, or positions 1-7 from the 3′ end, of the antisense strand are connected to adjacent nucleotides via phosphorothioate linkages.

The dsRNA compounds can be modified to improve stability in serum or in growth medium for cell cultures. In order to enhance the stability, the 3′-residues can be stabilized against degradation, e.g., they can be selected such that they consist of purine nucleotides, such as adenosine or guanosine nucleotides. Alternatively, substitution of pyrimidine nucleotides by modified analogues, e.g., substitution of uridine by 2′-deoxythymidine is tolerated and does not affect the efficiency of RNA interference.

In some embodiments, the present application features dsRNA that include first and second strands wherein the second strand and/or first strand is modified by the substitution of internal nucleotides with modified nucleotides, such that in vivo stability is enhanced as compared to a corresponding unmodified oligonucleotide. As defined herein, an “internal” nucleotide is one occurring at any position other than the 5′ end or 3′ end of nucleic acid molecule, polynucleotide or oligonucleotide. In one embodiment, the sense strand and/or antisense strand is modified by the substitution of at least one internal nucleotide. In another embodiment, the sense strand and/or antisense strand is modified by the substitution of at least 2, 3, 4, 5, 6, 7, 8, 9, 10, 11, 12, 13, 14, 15, 16, 17, 18, 19, 20, 21, 22, 23, 24, 25 or more internal nucleotides. In another embodiment, the sense strand and/or antisense strand is modified by the substitution of at least 5%, 10%, 15%, 20%, 25%, 30%, 35%, 40%, 45%, 50%, 55%, 60%, 65%, 70%, 75%, 80%, 85%, 90%, 95% or more of the internal nucleotides. In yet another embodiment, the sense strand and/or antisense strand is modified by the substitution of all of the internal nucleotides.

In one aspect, the present application features dsRNA compounds that are at least 80% chemically modified. In certain embodiments, the dsRNA compounds can be fully chemically modified, i.e., 100% of the nucleotides are chemically modified. In another aspect, the dsRNA compounds comprise 2′-OH ribose groups that are at least 80% chemically modified. In certain embodiments, the dsRNA compounds comprise 2′-OH ribose groups that are about 80%, 85%, 90%, 95%, or 100% chemically modified.

In certain embodiments, the dsRNA compounds can contain at least one modified nucleotide analogue. The nucleotide analogues can be located at positions where the target-specific silencing activity, such as the RNAi mediating activity or translational repression activity is not substantially affected (e.g., in a region at the 5′-end and/or the 3′-end of the siRNA molecule.

Exemplary nucleotide analogues include sugar- and/or backbone-modified ribonucleotides (i.e., include modifications to the phosphate-sugar backbone). For example, the phosphodiester linkages of natural RNA can be modified to include at least one of a nitrogen or sulfur heteroatom. In exemplary backbone-modified ribonucleotides, the phosphoester group connecting to adjacent ribonucleotides is replaced by a modified group, e.g., of phosphothioate group. In exemplary sugar-modified ribonucleotides, the 2′ OH-group is replaced by a group selected from H, OR, R, halo, SH, SR, NH₂, NHR, NR₂ or ON, wherein R is C1-C6 alkyl, alkenyl or alkynyl and halo is F, Cl, Br or I.

In certain embodiments, the modifications are 2′-fluoro, 2′-amino and/or 2′-thio modifications. Modifications include 2′-fluoro-cytidine, 2′-fluoro-uridine, 2′-fluoro-adenosine, 2′-fluoro-guanosine, 2′-amino-cytidine, 2′-amino-uridine, 2′-amino-adenosine, 2′-amino-guanosine, 2,6-diaminopurine, 4-thio-uridine, and/or 5-amino-allyl-uridine. In a certain embodiment, the 2′-fluoro ribonucleotides are every uridine and cytidine. Additional exemplary modifications include 5-bromo-uridine, 5-iodo-uridine, 5-methyl-cytidine, ribo-thymidine, 2-aminopurine, 2′-amino-butyryl-pyrene-uridine, 5-fluoro-cytidine, and 5-fluoro-uridine. 2′-deoxy-nucleotides and 2′-Ome nucleotides can also be used within modified RNA-silencing agents moieties of the instant invention. Additional modified residues include, deoxy-abasic, inosine, N3-methyl-uridine, N6,N6-dimethyl-adenosine, pseudouridine, purine ribonucleoside and ribavirin. In a certain embodiment, the 2′ moiety is a methyl group such that the linking moiety is a 2′-O-methyl oligonucleotide.

### Heavily Modified Oligonucleotides

In certain embodiments, the dsRNA compound, or a strand thereof, comprises at least 80% chemically modified nucleotides. In certain embodiments, the dsRNA compound is fully chemically modified, i.e., 100% of the nucleotides are chemically modified.

In certain embodiments, the dsRNA compound is 2′-O-methyl rich, i.e., comprises greater than 50% 2′-O-methyl content. In certain embodiments, the dsRNA compound comprises at least about 55%, 60%, 65%, 70%, 75%, 80%, 85%, 90%, 95%, or 100% 2′-O-methyl nucleotide content. In certain embodiments, dsRNA compound comprises at least about 70% 2′-O-methyl nucleotide modifications. In certain embodiments, the dsRNA compound comprises between about 70% and about 90% 2′-O-methyl nucleotide modifications. In certain embodiments, the dsRNA compound comprises an antisense strand and sense strand, and the antisense strand comprises at least about 70% 2′-O-methyl nucleotide modifications. In certain embodiments, the antisense strand comprises between about 70% and about 90% 2′-O-methyl nucleotide modifications. In certain embodiments, the sense strand comprises at least about 70% 2′-O-methyl nucleotide modifications. In certain embodiments, the sense strand comprises between about 70% and about 90% 2′-O-methyl nucleotide modifications. In certain embodiments, the sense strand comprises between 100% 2′-O-methyl nucleotide modifications.

2′-O-methyl rich dsRNA compounds and specific chemical modification patterns are further described in U.S. Ser. No. 16/550,076 (filed Aug. 23, 2019) and U.S. Ser. No. 62/891,185 (filed Aug. 23, 2019), each of which is incorporated herein by reference.

### Internucleotide Linkage Modifications

In certain embodiments, at least one internucleotide linkage, intersubunit linkage, or nucleotide backbone is modified in the oligonucleotide. In certain embodiments, all of the internucleotide linkages in the oligonucleotide are modified. In certain embodiments, the modified internucleotide linkage comprises a phosphorothioate internucleotide linkage. In certain embodiments, the oligonucleotide comprise 1, 2, 3, 4, 5, 6, 7, 8, 9, 10, 11, 12, 13, 14, 15, 16, 17, 18, 19, 20, 21, 22, 23, 24, or 25 phosphorothioate internucleotide linkages. In certain embodiments, the oligonucleotide comprises 4-16 phosphorothioate internucleotide linkages. In certain embodiments, the oligonucleotide comprises 8-13 phosphorothioate internucleotide linkages. In certain embodiments, the oligonucleotide is a dsRNA comprising an antisense strand and a sense strand, each comprising a 5′ end and a 3′ end. In certain embodiments, the nucleotides at positions 1 and 2 from the 5′ end of sense strand are connected to adjacent ribonucleotides via phosphorothioate internucleotide linkages. In certain embodiments, the nucleotides at positions 1 and 2 from the 3′ end of sense strand are connected to adjacent ribonucleotides via phosphorothioate internucleotide linkages. In certain embodiments, the nucleotides at positions 1 and 2 from the 5′ end of antisense strand are connected to adjacent ribonucleotides via phosphorothioate internucleotide linkages. In certain embodiments, the nucleotides at positions 1-2 to 1-8 from the 3′ end of antisense strand are connected to adjacent ribonucleotides via phosphorothioate internucleotide linkages. In certain embodiments, the nucleotides at positions 1-2, 1-3, 1-4, 1-5, 1-6, 1-7, or 1-8 from the 3′ end of antisense strand are connected to adjacent ribonucleotides via phosphorothioate internucleotide linkages. In certain embodiments, the nucleotides at positions 1-2 to 1-7 from the 3′ end of antisense strand are connected to adjacent ribonucleotides via phosphorothioate internucleotide linkages.

In certain embodiments, an oligonucleotide can include an internucleotide linkage represented by formula (IV):

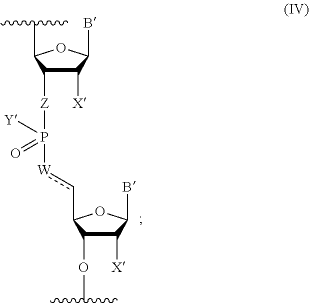

wherein:
B′ is a base pairing moiety;
W is selected from the group consisting of O, OCH₂, OCH, CH₂, and CH, or W is O or O(CH₂)ₙ;
X′ is selected from the group consisting of H, halo, hydroxy, OR, F, SH, SR, NR²₂ and C1-C6-alkoxy;
Y′ is selected from the group consisting of O⁻, OH, OR, OR², NH⁻, NH₂, NR²₂, BH₃, S⁻, R¹, and SH;
Z is selected from the group consisting of O, CH₂ or O(CH);
R is a protecting group;
R¹ is alkyl, allyl or aryl; and
R² is alkyl, allyl or aryl,
n is 1 to 10, and
[symbol] is an optional double bond.

Suitable protecting groups are known in the art. In certain embodiments, R can be selected from the group consisting of dimethoxytrityl (DMTr), succinate, tert-butyl dimethylsilyl (TBDMS), benzoyl (Bz), benzyl (Bn), methoxyethoxymethyl ether (MOM), methoxybenzyl ether (PMB), methylthiomethyl ether, pivaloyl (Piv), tetrahydropyranyl (THP), tetrahydrofuranyl (THF), trityl (Trt), triisopropylsilyl (TIPS), tert-butyldiphenylsilyl (TBDPS), and acetate.

### Tethered Ligands

The present disclosure also includes an oligonucleotide which is tethered to another moiety (e.g. other than a hydrophobic moiety, such as a non-nucleic acid moiety (e.g., a peptide), an organic compound (e.g., a dye), or the like). For example, a ligand can be tethered to an conjugated oligonucleotide to improve stability, hybridization thermodynamics with a target nucleic acid, targeting to a particular tissue or cell-type, or cell permeability, e.g., by an endocytosis-dependent or -independent mechanism. Ligands and associated modifications can also increase sequence specificity and consequently decrease off-site targeting. A tethered ligand can include one or more modified bases or sugars that can function as intercalators. These are preferably located in an internal region, such as in a bulge of oligonucleotide/target duplex. The intercalator can be an aromatic, e.g., a polycyclic aromatic or heterocyclic aromatic compound. A polycyclic intercalator can have stacking capabilities, and can include systems with 2, 3, or 4 fused rings. The universal bases described herein can be included on a ligand. In one embodiment, the ligand can include a cleaving group that contributes to target gene inhibition by cleavage of the target nucleic acid. The cleaving group can be, for example, a bleomycin (e.g., bleomycin-A5, bleomycin-A2, or bleomycin-B2), pyrene, phenanthroline (e.g., O-phenanthroline), a polyamine, a tripeptide (e.g., lys-tyr-lys tripeptide), or metal ion chelating group. The metal ion chelating group can include, e.g., an Lu(III) or EU(III) macrocyclic complex, a Zn(II) 2,9-dimethylphenanthroline derivative, a Cu(II) terpyridine, or acridine, which can promote the selective cleavage of target RNA at the site of the bulge by free metal ions, such as Lu(III). In some embodiments, a peptide ligand can be tethered to a conjugated oligonucleotide to promote cleavage of the target RNA, e.g., at the bulge region. For example, 1,8-dimethyl-1,3,6,8,10,13-hexaazacyclotetradecane (cyclam) can be conjugated to a peptide (e.g., by an amino acid derivative) to promote target RNA cleavage. A tethered ligand can be an aminoglycoside ligand, which can cause an oligonucleotide to have improved hybridization properties or improved sequence specificity. Exemplary aminoglycosides include glycosylated polylysine, galactosylated polylysine, neomycin B, tobramycin, kanamycin A, and acridine conjugates of aminoglycosides, such as Neo-N-acridine, Neo-S-acridine, Neo-C-acridine, Tobra-N-acridine, and KanaA-N-acridine. Use of an acridine analog can increase sequence specificity. For example, neomycin B has a high affinity for RNA as compared to DNA, but low sequence-specificity. An acridine analog, neo-5-acridine has an increased affinity for the HIV Rev-response element (RRE). In some embodiments the guanidine analog (the guanidinoglycoside) of an aminoglycoside ligand is tethered to a conjugated oligonucleotide. In a guanidinoglycoside, the amine group on the amino acid is exchanged for a guanidine group. Attachment of a guanidine analog can enhance cell permeability of a conjugated oligonucleotide. A tethered ligand can be a poly-arginine peptide, peptoid or peptidomimetic, which can enhance the cellular uptake of a conjugated oligonucleotide.

Exemplary ligands are coupled, preferably covalently, either directly or indirectly via an intervening tether, to a ligand-conjugated carrier. In exemplary embodiments, the ligand is attached to the carrier via an intervening tether. In exemplary embodiments, a ligand alters the distribution, targeting or lifetime of a conjugated oligonucleotide into which it is incorporated. In exemplary embodiments, a ligand provides an enhanced affinity for a selected target, e.g., molecule, cell or cell type, compartment, e.g., a cellular or organ compartment, tissue, organ or region of the body, as, e.g., compared to a species absent such a ligand.

Exemplary ligands can improve transport, hybridization, and specificity properties and can improve nuclease resistance of the resultant natural or modified oligonucleotide, or a. Ligands in general can include therapeutic modifiers, e.g., for enhancing uptake; diagnostic compounds or reporter groups e.g., for monitoring distribution; cross-linking agents; nuclease-resistance conferring moieties; and natural or unusual nucleobases. General examples include lipophiles, lipids, steroids (e.g., uvaol, hecigenin, diosgenin), terpenes (e.g., triterpenes, e.g., sarsasapogenin, Friedelin, epifriedelanol derivatized lithocholic acid), vitamins (e.g., folic acid, vitamin A, biotin, pyridoxal), carbohydrates, proteins, protein binding agents, integrin targeting molecules, polycationics, peptides, polyamines, and peptide mimics. Ligands can include a naturally occurring substance, (e.g., human serum albumin (HSA), low-density lipoprotein (LDL), or globulin); carbohydrate (e.g., a dextran, pullulan, chitin, chitosan, inulin, cyclodextrin or hyaluronic acid); amino acid, or a lipid. The ligand can also be a recombinant or synthetic molecule, such as a synthetic polymer, e.g., a synthetic polyamino acid. Examples of polyamino acids include polyamino acid is a polylysine (PLL), poly L-aspartic acid, poly L-glutamic acid, styrene-maleic acid anhydride copolymer, poly(L-lactide-co-glycolide) copolymer, divinyl ether-maleic anhydride copolymer, N-(2-hydroxypropyl)methacrylamide copolymer (HMPA), polyethylene glycol (PEG), polyvinyl alcohol (PVA), polyurethane, poly(2-ethylacrylic acid), N-isopropylacrylamide polymers, or polyphosphazine. Example of polyamines include: polyethylenimine, polylysine (PLL), spermine, spermidine, polyamine, pseudopeptide-polyamine, peptidomimetic polyamine, dendrimer polyamine, arginine, amidine, protamine, cationic lipid, cationic porphyrin, quaternary salt of a polyamine, or an alpha helical peptide.

Ligands can also include targeting groups, e.g., a cell or tissue targeting agent, e.g., a lectin, glycoprotein, lipid or protein, e.g., an antibody, that binds to a specified cell type such as a kidney cell. A targeting group can be a thyrotropin, melanotropin, lectin, glycoprotein, surfactant protein A, mucin carbohydrate, multivalent lactose, multivalent galactose, N-acetyl-galactosamine, N-acetyl-glucosamine, multivalent mannose, multivalent fucose, glycosylated polyaminoacids, multivalent galactose, transferrin, bisphosphonate, polyglutamate, polyaspartate, a lipid, cholesterol, a steroid, bile acid, folate, vitamin B12, biotin, or an RGD peptide or RGD peptide mimetic. Other examples of ligands include dyes, intercalating agents (e.g. acridines and substituted acridines), cross-linkers (e.g. psoralene, mitomycin C), porphyrins (TPPC4, texaphyrin, Sapphyrin), polycyclic aromatic hydrocarbons (e.g., phenazine, dihydrophenazine, phenanthroline, pyrenes), lys-tyr-lys tripeptide, aminoglycosides, guanidium aminoglycodies, artificial endonucleases (e.g. EDTA), lipophilic molecules, e.g., cholesterol (and thio analogs thereof), cholic acid, cholanic acid, lithocholic acid, adamantane acetic acid, 1-pyrene butyric acid, dihydrotestosterone, glycerol (e.g., esters (e.g., mono, bis, or tris fatty acid esters, e.g., C10, C11, C12, C13, C14, C15, C16, C17, C18, C19, or C20 fatty acids) and ethers thereof, e.g., C10, C11, C12, C13, C14, C15, C16, C17, C18, C19, or C20 alkyl; e.g., 1,3-bis-O(hexadecyl)glycerol, 1,3-bis-O(octaadecyl)glycerol), geranyloxyhexyl group, hexadecylglycerol, borneol, menthol, 1,3-propanediol, heptadecyl group, palmitic acid, stearic acid (e.g., glyceryl distearate), oleic acid, myristic acid, 03-(oleoyl)lithocholic acid, 03-(oleoyl)cholenic acid, dimethoxytrityl, or phenoxazine) and peptide conjugates (e.g., antennapedia peptide, Tat peptide), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, naproxen, vitamin E, folic acid), synthetic ribonucleases (e.g., imidazole, bisimidazole, histamine, imidazole clusters, acridine-imidazole conjugates, Eu³⁺ complexes of tetraazamacrocycles), dinitrophenyl, HRP or AP.

Ligands can be proteins, e.g., glycoproteins, or peptides, e.g., molecules having a specific affinity for a co-ligand, or antibodies e.g., an antibody, that binds to a specified cell type such as a cancer cell, endothelial cell, or bone cell. Ligands can also include hormones and hormone receptors. They can also include non-peptidic species, such as lipids, lectins, carbohydrates, vitamins, cofactors, multivalent lactose, multivalent galactose, N-acetyl-galactosamine, N-acetyl-glucosamine multivalent mannose, or multivalent fucose. The ligand can be, for example, a lipopolysaccharide, an activator of p38 MAP kinase, or an activator of NF-κB.

The ligand can be a substance, e.g., a drug, which can increase the uptake of the conjugated oligonucleotide into the cell, for example, by disrupting the cell's cytoskeleton, e.g., by disrupting the cell's microtubules, microfilaments, and/or intermediate filaments. The drug can be, for example, taxon, vincristine, vinblastine, cytochalasin, nocodazole, japlakinolide, latrunculin A, phalloidin, swinholide A, indanocine, or myoservin. The ligand can increase the uptake of the conjugated oligonucleotide into the cell by activating an inflammatory response, for example. Exemplary ligands that would have such an effect include tumor necrosis factor alpha (TNFα), interleukin-1 beta, or gamma interferon. In one aspect, the ligand is a lipid or lipid-based molecule. Such a lipid or lipid-based molecule preferably binds a serum protein, e.g., human serum albumin (HSA).

In another aspect, the ligand is a moiety, e.g., a vitamin, which is taken up by a target cell, e.g., a proliferating cell. These are particularly useful for treating disorders characterized by unwanted cell proliferation, e.g., of the malignant or non-malignant type, e.g., cancer cells. Exemplary vitamins include vitamin A, E, and K. Other exemplary vitamins include are B vitamin, e.g., folic acid, B12, riboflavin, biotin, pyridoxal or other vitamins or nutrients taken up by cancer cells.

In another aspect, the ligand is a cell-permeation agent, preferably a helical cell-permeation agent. Preferably, the agent is amphipathic. An exemplary agent is a peptide such as tat or antennopedia. If the agent is a peptide, it can be modified, including a peptidylmimetic, invertomers, non-peptide or pseudo-peptide linkages, and use of D-amino acids. The helical agent is preferably an alpha-helical agent, which preferably has a lipophilic and a lipophobic phase.

The ligand can be a peptide or peptidomimetic. A peptidomimetic (also referred to herein as an oligopeptidomimetic) is a molecule capable of folding into a defined three-dimensional structure similar to a natural peptide. The attachment of peptide and peptidomimetics to oligonucleotide agents can affect pharmacokinetic distribution of the conjugated oligonucleotide, such as by enhancing cellular recognition and absorption. The peptide or peptidomimetic moiety can be about 5-50 amino acids long, e.g., about 5, 10, 15, 20, 25, 30, 35, 40, 45, or 50 amino acids long. A peptide or peptidomimetic can be, for example, a cell permeation peptide, cationic peptide, amphipathic peptide, or hydrophobic peptide (e.g., consisting primarily of Tyr, Trp or Phe). The peptide moiety can be a dendrimer peptide, constrained peptide or crosslinked peptide. The peptide moiety can be an L-peptide or D-peptide. In another alternative, the peptide moiety can include a hydrophobic membrane translocation sequence (MTS). A peptide or peptidomimetic can be encoded by a random sequence of DNA, such as a peptide identified from a phage-display library, or one-bead-one-compound (OBOC) combinatorial library (Lam et al., Nature 354:82-84, 1991). In exemplary embodiments, the peptide or peptidomimetic tethered to a conjugated oligonucleotide via an incorporated monomer unit is a cell targeting peptide such as an arginine-glycine-aspartic acid (RGD)-peptide, or RGD mimic. A peptide moiety can range in length from about 5 amino acids to about 40 amino acids. The peptide moieties can have a structural modification, such as to increase stability or direct conformational properties. Any of the structural modifications described below can be utilized.

### Pharmaceutical Compositions and Methods of Administration

In one aspect, a pharmaceutical composition can include a therapeutically effective amount of one or more conjugates as described herein, and a pharmaceutically acceptable carrier. In one embodiment, the pharmaceutical composition comprises one or more conjugated oligonucleotides as described herein, and a pharmaceutically acceptable carrier. In a particular embodiment, the pharmaceutical composition comprises one conjugated oligonucleotide as described herein, and a pharmaceutically acceptable carrier. In another particular embodiment, the pharmaceutical composition comprises a combination of two conjugated oligonucleotides as described herein, and a pharmaceutically acceptable carrier.

It is understood that certain internucleotide linkages provided herein, including, e.g., phosphodiester and phosphorothioate, comprise a formal charge of −1 at physiological pH, and that the formal charge can be balanced by a cationic moiety, e.g., an alkali metal such as sodium or potassium, an alkali earth metal such as calcium or magnesium, or an ammonium or guanidinium ion.

The present disclosure provides uses of the above-described agents for prophylactic and/or therapeutic treatments. Accordingly, the conjugated oligonucleotides of the present disclosure can be incorporated as active agents into pharmaceutical compositions suitable for administration. Such compositions typically comprise the conjugate and a pharmaceutically acceptable carrier. The use of such media and agents for pharmaceutically active substances is well known in the art. Except insofar as any conventional media or agent is incompatible with the conjugated oligonucleotides, use thereof in the compositions is contemplated. Supplementary active compounds can also be incorporated into the compositions.

A pharmaceutical composition of the present disclosure is formulated to be compatible with its intended route of administration. Examples of routes of administration include parenteral, e.g., intravenous (IV), intradermal, subcutaneous (SC or SQ), intraperitoneal, intramuscular, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates and agents for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor ELM (BASF, Parsippany, N.J.) or phosphate buffered saline (PBS). The composition can be sterile and fluid for syringability. The composition must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the conjugate in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, the preferred methods of preparation are vacuum drying and freeze-drying which yields a powder of the active ingredient plus any additional desired ingredient from a previously sterile-filtered solution thereof.

Toxicity and therapeutic efficacy of a conjugated oligonucleotide can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀.

The data obtained from the cell culture assays and animal studies can be used in formulating a range of dosage for use in humans. The dosage of such compounds lies preferably within a range of circulating concentrations that include the ED₅₀ with little or no toxicity. The dosage can vary within this range depending upon the dosage form employed and the route of administration utilized. For any conjugated oligonucleotide used in the method of the present disclosure, the therapeutically effective dose can be estimated initially from cell culture assays. A dose can be formulated in animal models to achieve a circulating plasma concentration range that includes the EC₅₀ (i.e., the concentration of the test compound which achieves a half-maximal response) as determined in cell culture. Such information can be used to determine useful doses in humans more accurately. Levels in plasma can be measured, for example, by high performance liquid chromatography.

### Methods of Treatment

In one aspect, provided herein is a method for selectively delivering a conjugated oligonucleotide of formula (I) or (I_{A}), or a disclosed embodiment thereof, to a particular organ in a subject (e.g., a patient), comprising administering said compound to the subject, wherein the conjugated oligonucleotide selectively accumulates in one or more extrahepatic tissues or organs. In certain embodiments, a conjugated oligonucleotide selectively accumulates in one or more of heart, kidneys, muscle, lung, urinary bladder, pancreas, duodenum, spleen, adrenal gland, reproductive, extra-embryonic and fat tissue in the subject. In certain embodiments, a conjugated oligonucleotide of the present disclosure selectively accumulates in at least one of heart, muscle or lung tissue.

A conjugated oligonucleotide can be administered at a unit dose less than about 1.4 mg per kg of bodyweight, or less than 10, 5, 2, 1, 0.5, 0.1, 0.05, 0.01, 0.005, 0.001, 0.0005, 0.0001, 0.00005 or 0.00001 mg per kg of bodyweight, and less than 200 nmole of conjugated oligonucleotide (e.g., about 4.4×10¹⁶ copies) per kg of bodyweight, or less than 1500, 750, 300, 150, 75, 15, 7.5, 1.5, 0.75, 0.15, 0.075, 0.015, 0.0075, 0.0015, 0.00075, 0.00015 nmole of the conjugated oligonucleotide per kg of bodyweight. The unit dose, for example, can be administered by injection (e.g., intravenous or intramuscular, intrathecally, or directly into the brain), an inhaled dose, or a topical application. Particularly preferred dosages are less than 2, 1, or 0.1 mg/kg of body weight.

Delivery of a conjugated oligonucleotide can include systemic administration and local administration (e.g., the skin, central nervous system, eye or lung). Delivery of a conjugated oligonucleotide directly to an organ (e.g., directly to the brain, spinal column, placenta, liver and/or kidneys) can be at a dosage on the order of about 0.00001 mg to about 3 mg per organ, or preferably about 0.0001-0.001 mg per organ, about 0.03-3.0 mg per organ, about 0.1-3.0 mg per eye or about 0.3-3.0 mg per organ. The dosage can be an amount effective to treat or prevent a neurological disease or disorder (e.g., Huntington's disease) or a liver-, kidney- or pregnancy-related disease or disorder (e.g., PE, postpartum PE, eclampsia and/or HELLP). In one embodiment, the unit dose is administered less frequently than once a day, e.g., less than every 2, 4, 8 or 30 days. In another embodiment, the unit dose is not administered with a frequency (e.g., not a regular frequency). For example, the unit dose can be administered a single time. In one embodiment, the effective dose is administered with other traditional therapeutic modalities.

In one embodiment, a subject is administered an initial dose, and one or more maintenance doses of a conjugated oligonucleotide. The maintenance dose or doses are generally lower than the initial dose, e.g., one-half less of the initial dose. A maintenance regimen can include treating the subject with a dose or doses ranging from 0.01 μg to 1.4 mg/kg of body weight per day, e.g., 10, 1, 0.1, 0.01, 0.001, or 0.00001 mg per kg of bodyweight per day. The maintenance doses are preferably administered no more than once every 5, 10, or 30 days. Further, the treatment regimen can last for a period of time which will vary depending upon the nature of the particular disease, its severity and the overall condition of the patient. The dosage can be delivered no more than once per day, e.g., no more than once per 24, 36, 48, or more hours, e.g., no more than once every 5 or 8 days. Following treatment, the patient can be monitored for changes in his condition and for alleviation of the symptoms of the disease state. The dosage of the conjugate can either be increased in the event the patient does not respond significantly to current dosage levels, or the dose can be decreased if an alleviation of the symptoms of the disease state is observed, if the disease state has been ablated, or if undesired side-effects are observed.

### EXAMPLES

### Materials and Methods

### General Methods for Oligonucleotide Synthesis, Deprotection and Purification

Oligonucleotides were synthesized using standard phosphoramidite based solid-phase synthesis, deprotected and purified by HPLC. Custom made lipid functionalized controlled pore glass (CPG) was used for the synthesis of sense strands (see supplemental information). Antisense strands were synthesized on UnyLinker functionalized CPG (ChemGenes, Co.). Standard deprotection and cleavage conditions specific to sense strands or antisense strands were used. All siRNA synthesized were fully modified with 2′-O-methyl or 2′-fluoro groups, phosphorothioate backbones in the termini and 5′-(E)-vinylphosphonate on the antisense strand. See Table 1 for the sequence and modifications of the antisense and sense strands used in this study. Oligonucleotides were purified by standard HPLC methods specific to sense and antisense strands. The identity of custom synthesized lipid conjugates precursors was verified using NMR and LC-MS and the identity and quality of synthesized oligonucleotides were verified using LC-MS.

All chemical reactions were performed under argon atmosphere using anhydrous freshly distilled solvents unless otherwise stated. Dichloromethane (DCM), acetonitrile (ACN) and dimethylformamide (DMF) were dried using a PureSolv MD 5× Channel Solvent Purification System, tested with Karl Fisher titration and stored on molecular sieves. The NMR spectra were recorded using Brucker 500 MHz spectrometer. ¹H, ¹³C, and ³¹P NMR spectra were recorded at 500 MHz (¹H NMR, 500 MHz; ¹³C-NMR, 126 MHz; ³¹P-NMR, 202 MHz). The chemical shifts were measured from tetramethylsilane (0 ppm), CDCl₃ (7.26 ppm), DMSO-d₆ (2.49 ppm) and CD₃CN-d₃ (1.93 ppm) for ¹H-NMR spectra, CDCl₃ (77.0 ppm), DMSO-d₆ (39.7 ppm) and CD₃CN-d₃ (1.30 ppm) for ¹³C-NMR spectra, and 85% H₃PO₄ for ³¹P-NMR spectra as external standards. High-resolution electrospray ionization mass spectrometry (HR-ESI-MS) analysis for the compounds were performed on a Thermo Scientific Orbitrap Velos Pro mass spectrometer in the positive ion mode. The mass analysis of oligonucleotides was conducted by LC-MS on an Agilent 6530 accurate-mass Q-TOF LC/MS (Agilent technologies, Santa Clara, CA). Flash chromatography was performed using Teledyne Isco CombiFlash Rf system and prepacked (silica gel, 40-60 μm) columns purchased from Bonna-Agela Technologies (Tianjin, China). Analytical thin-layer chromatography (TLC) was performed using silica gel 60 F₂₅₄ using UV light as the visualizing agent. The synthesis of modified oligonucleotides was performed using MerMaid-12 DNA/RNA synthesizer (Bio automation, USA). Analytical reverse phase HPLC were performed on Agilent 1260 Infinity Analytical SFC System combined with an Agilent 1100 series quaternary pump with a degasser. Purified oligonucleotides were desalted by Sephadex G-25 (GE Healthcare). N⁻-Fmoc-L-serine tert-butyl ester and choline p-toluenesulfonate were from Tokyo Chemical Industry (Portland, OR), Alfa Aesar (Ward Hill, MA) respectively. The rest of the chemicals and solvents were obtained from either Sigma (St. Louis, MO) or Fisher Scientific (Waltham, MA). RNA synthesis reagents, 2′-cyanoethyl-N,N-diisopropylchlorophosphoramidite and controlled pore glass (CPG) functionalized by a C7 linker were obtained from ChemGenes (Wilmington, MA).

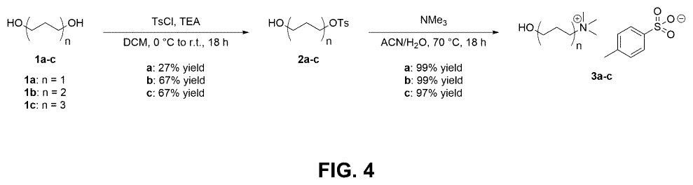

1. Synthesis of Choline p-toluenesulfonate Derivatives 3a-c (Scheme S1 (FIG. 4)):

3-hydroxypropyl p-toluenesulfonate 2a. 1,3-propanediol (22.7 mL, 314.7 mmol, 5.0 equiv.) was dissolved in CH₂Cl₂ (125.7 mL) and then TEA (9.66 mL, 69.3 mmol, 1.1 equiv.) was added to the solution under argon. This mixture was placed in an ice bath. Separately, tosyl chloride (12 g, 62.9 mmol, 1.0 equiv.) was dissolved in CH₂Cl₂ (94.2 mL). The tosyl chloride solution was then dripped into the above 1,3-propanediol mixture over 30 min, and the reaction was stirred overnight for 18 h, warming to room temperature. After reaching completion, the solvent was removed by rotary evaporation. The crude was then purified by column chromatography on silica gel using AcOEt/Hexane (6/4) as eluent, to afford 2a as a viscous oil (3.93 g, 17.1 mmol, yield 27%).

6-hydroxyhexyl p-toluenesulfonate 2b. 1,6-hexanediol (12.4 g, 104.9 mmol, 5.0 equiv.) was dissolved in CH₂Cl₂ (41.9 mL) and then TEA (3.22 mL, 23.1 mmol, 1.1 equiv.) was added to the solution under argon. This mixture was placed in an ice bath. Separately, tosyl chloride (4 g, 21.0 mmol, 1.0 equiv.) was dissolved in CH₂Cl₂ (31.4 mL). The tosyl chloride solution was then dripped into the above 1,6-hexaediol mixture over 30 min, and the reaction was stirred overnight for 18 h, warming to room temperature. After reaching completion, the solvent was removed by rotary evaporation. The crude was then purified by column chromatography on silica gel using AcOEt/Hexane (2/8) as eluent, to afford 2b as a viscous oil (3.85 g, 14.1 mmol, yield 67%).

9-hydroxynonyl p-toluenesulfonate 2c. 1,9-nonanediol (21.0 g, 131.1 mmol, 5.0 equiv.) was dissolved in CH₂Cl₂ (52.5 mL) and then TEA (4.02 mL, 28.9 mmol, 1.1 equiv.) was added to the solution under argon. This mixture was placed in an ice bath. Separately, tosyl chloride (5 g, 26.2 mmol, 1.0 equiv.) was dissolved in CH₂Cl₂ (39.3 mL). The tosyl chloride solution was then dripped into the above 1,9-nonanediol mixture over 30 min, and the reaction was stirred overnight for 18 h, warming to room temperature. After reaching completion, the reaction mixture was washed with water and brine. The aqueous was extracted with DCM. The organic phase was dried on magnesium sulfate, filtrated and evaporated under vacuum. The crude was then purified by column chromatography on silica gel using a gradient of AcOEt in Hexane (50-60%) as eluent, to afford 2c as a viscous oil (5.56 g, 17.7 mmol, yield 67%).

3-hydroxy-n,n,n-trimethylpropane-1-ammonium p-toluenesulfonate 3a. To a solution of 2a (2.76 g, 12.0 mmol, 1.0 equiv.) in ACN (119.9 mL) was added trimethylamine (45% in water) (3.15 mL, 24.0 mmol, 2.0 equiv.). After 18 h at 70° C., the solvent was removed under vacuum and the crude was dried by co-evaporation with dry toluene and dry ACN, to afford 3a as a white solid (3.42 g, 11.8 mmol, yield 99%).

6-hydroxy-n,n,n-trimethylhexane-1-ammonium p-toluenesulfonate 3b. To a solution of 2b (3.85 g, 14.1 mmol, 1.0 equiv.) in ACN (141.4 mL) was added trimethylamine (45% in water) (3.71 mL, 28.3 mmol, 2.0 equiv.). After 18 h at 70° C., the solvent was removed under vacuum and the crude was dried by co-evaporation with dry toluene and dry ACN, to afford 3b as a white solid (4.63 g, 14.0 mmol, yield 99%):

¹H NMR (500 MHz, DMSO-d₆) σ_{H} (ppm) 7.47 (d, J=5.0 Hz, 2H, Ar tosylate); 7.11 (d, J=5.0 Hz, 1H, Ar tosylate); 4.42-4.34 (t, J=5.0 Hz, 1H, OH); 3.44-3.36 (m, 2H, CH₂); 3.29-3.21 (m, 2H, CH₂); 3.03 (s, 9H, 3CH₃); 2.29 (s, 3H, CH₃ tosylate); 1.72-1.62 (m, 2H, CH₂); 1.47-1.40 (m, 2H, CH₂); 1.38-1.31 (m, 2H, CH₂); 1.30-1.23 (m, 2H, CH₂). ¹³C NMR (100 MHz, DMSO-d₆) σ_{c} (ppm) 146.33, 137.98 (Cq tosylate); 128.48 (CH Ar tosylate); 125.95 (CH Ar tosylate); 65.74 (CH₂); 60.94 (CH₂); 52.59 (3CH₃); 32.63 (CH₂); 25.48 (CH₂); 26.10 (CH₂); 22.53 (CH₂); 21.24 (CH₃ tosylate). HRMS (ESI⁺) m/z for calculated C₉H₂₂NO⁺ [M]⁺160.1696;

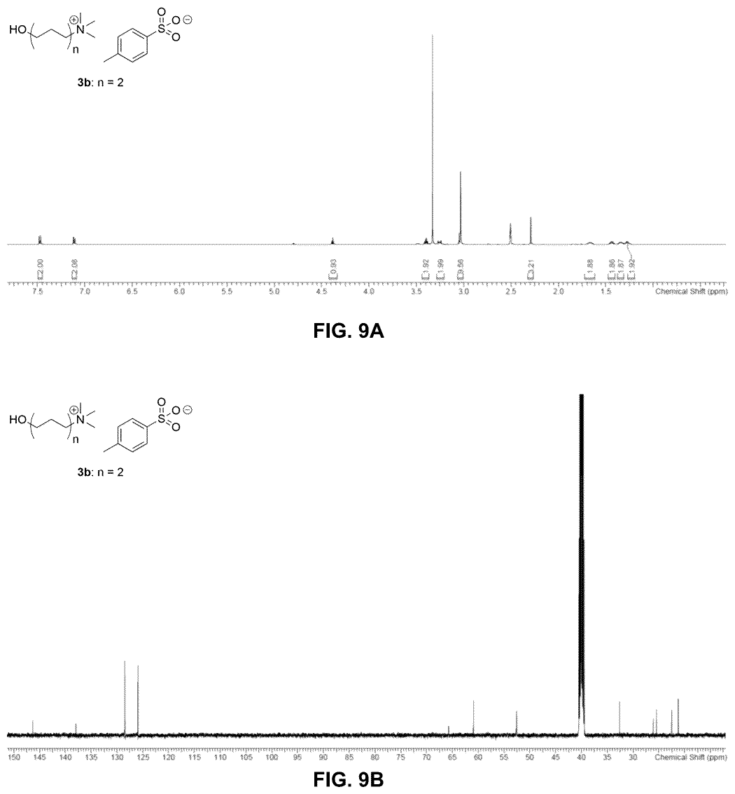

Found 160.1687. NMR spectra shown in FIGS. 9A-B.

9-hydroxy-n,n,n-trimethylnonane-1-ammonium p-toluenesulfonate 3c. To a solution of 2c (5.56 g, 17.7 mmol, 1.0 equiv.) in ACN (176.8 mL) was added trimethylamine (45% in water) (4.65 mL, 35.4 mmol, 2.0 equiv.). After 18 h at 70° C., the solvent was removed under vacuum and the crude was dried by co-evaporation with dry toluene and dry ACN, to afford 3c as a white solid (6.42 g, 17.2 mmol, yield 97%):

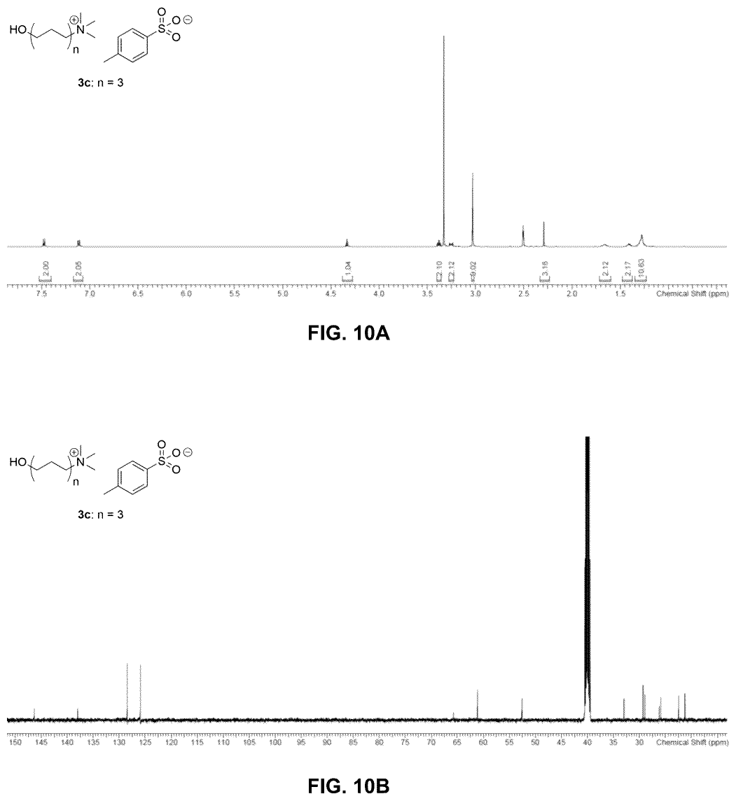

¹H NMR (500 MHz, DMSO-d₆) σ_{H} (ppm) 7.46 (d, J=5.0 Hz, 2H, Ar tosylate); 7.12 (d, J=5.0 Hz, 1H, Ar tosylate); 4.38-4.27 (t, J=5.0 Hz, 1H, OH); 3.40-3.36 (m, 2H, CH₂); 3.28-3.23 (m, 2H, CH₂); 3.03 (s, 9H, 3CH₃); 2.28 (s, 3H, CH₃ tosylate); 1.72-1.60 (m, 2H, CH₂); 1.47-1.37 (m, 2H, CH₂); 1.34-1.23 (m, 10H, 5CH₂). ¹³C NMR (100 MHz, DMSO-d₆) σ_{c} (ppm) 146.33, 137.97 (Cq tosylate); 128.48 (CH Ar tosylate); 125.95 (CH Ar tosylate); 65.76 (CH₂); 61.14 (CH₂); 52.58 (3CH₃); 32.96 (2*CH₂); 29.30, 28.91, 26.20, 25.92 (4*CH₂); 22.47 (CH₂); 21.24 (CH₃ tosylate). HRMS (ESI⁺) m/z for calculated C₁₂H₂₈NO⁺ [M]⁺202.2165; Found 202.2155. NMR spectra shown in FIGS. 10A-B.

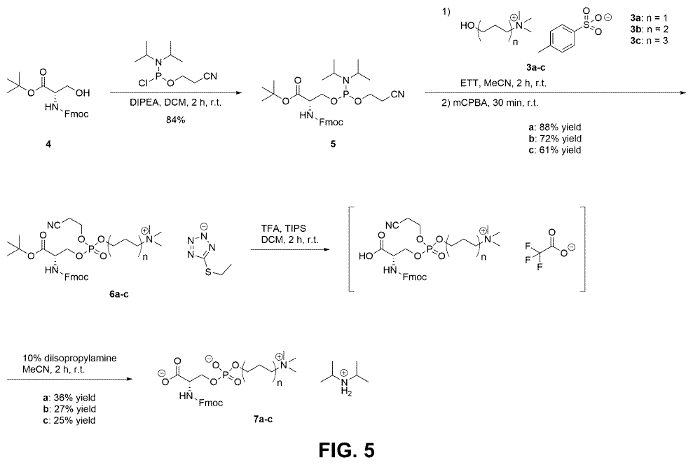

### 2. Synthesis of Phosphatidylcholine Moiety Derivatives 7a-c is Shown in FIG. 5 (Scheme 2)

Compound 5. Compound 4 (15.0 g, 39.1 mmol, 1.0 equiv.) was first dried by co-evaporation with dry toluene and dry DCM. Dry DCM (97.8 mL) and DIPEA (11.6 mL, 66.5 mmol, 1.7 equiv.) were added under argon and 2′-cyanoethyl-N,N-diisopropylchlorophosphoramidite (11.3 mL, 50.9 mmol, 1.3 equiv.) was added slowly via a syringe. The reaction mixture was stirred 2 h at room temperature. After reaching completion, the reaction mixture was washed with a solution of sodium bicarbonate and brine. The aqueous phase was extracted with DCM. The organic phase was dried on magnesium sulfate, filtrated and evaporated under vacuum. The crude mixture was then purified by column chromatography on silica gel using a gradient of AcOEt in Hexane (10-20%) as eluent, to afford 5 as a viscous oil (19.2 g, 32.9 mmol, yield 84%).

Compound 6a. Compound 5 (3.91 g, 6.70 mmol, 1.0 equiv.) and compound 3a (2.13 g, 7.36 mmol, 1.1 equiv.) was first dried by co-evaporation with dry toluene and dry ACN. Dry ACN was added under argon and ETT (0.25 M in ACN) (26.8 mL, 6.70 mmol, 1.0 equiv.) was added slowly with a syringe. The mixture was stirred 2 h at room temperature. After reaching completion, the reaction mixture was quenched with methanol (540 μL). Meta-chloroperoxybenzoic acid (mCPBA) (2.31 g, 13.4 mmol, 2.0 equiv.) was added by portion to the mixture. After 30 min of stirring, the mixture was reduced under vacuum. The crude was then purified by column chromatography on silica gel using a gradient of MeOH in DCM (0-30%) as eluent, to afford 6a as a mixture of tetrazolium (major counter anion) and tosylate salts (4.40 g, 5.90 mmol, yield 88%).

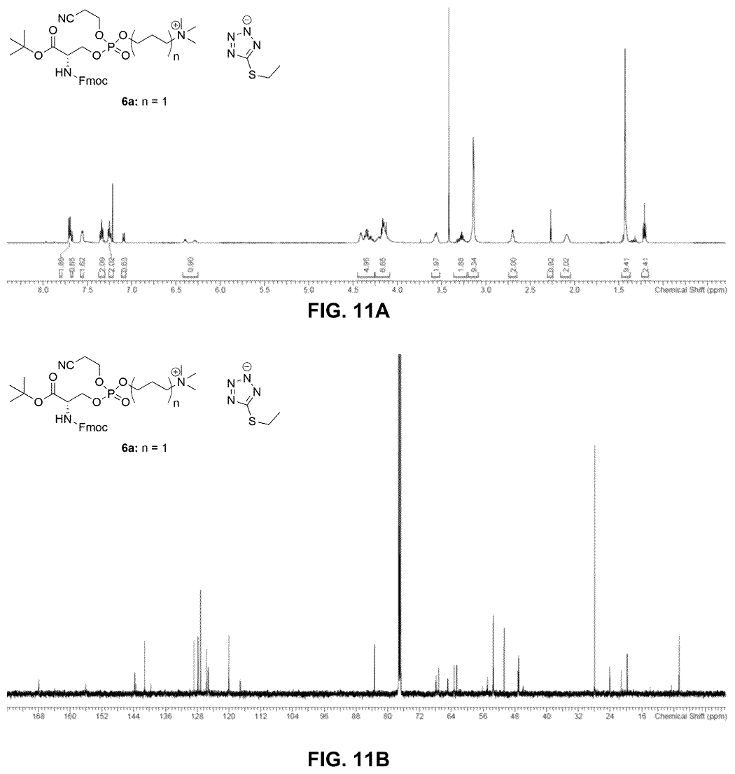

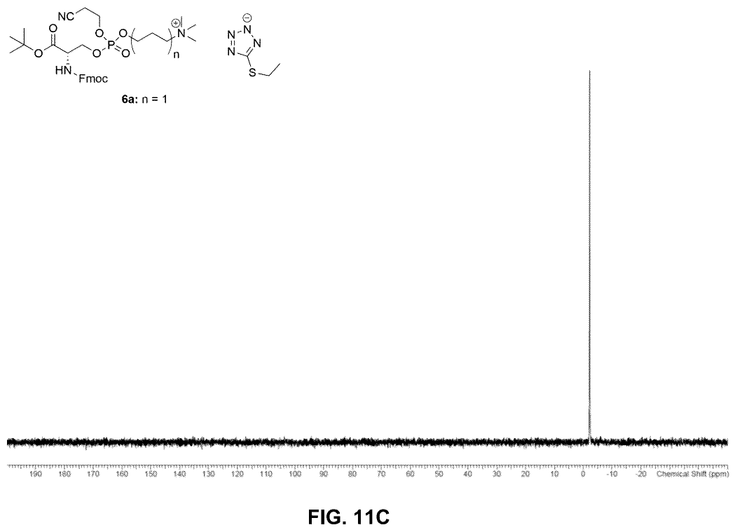

¹H NMR (500 MHz, CDCl₃) σ_{H} (ppm) 7.70 (d, J=10.0 Hz, 2H, Ar Fmoc); 7.68 (d, J=10.0 Hz, 1H, Ar tosylate); 7.56 (m, 2H, Ar Fmoc); 7.34 (t, J=10.0 Hz, 2H, Ar Fmoc); 7.26 (t, J=10.0 Hz, 2H, Ar Fmoc); 7.09 (d, J=10.0 Hz, 1H, Ar tosylate); 6.42-6.25 (dd, J=55.0 Hz, J=5.0 Hz, 1H, NH); 4.45-4.26 (m, 5H, CH Fmoc+CH₂+CH); 4.25-4.09 (m, 7H, 2*CH Fmoc+CH₂ C3 carbon chain+CH₂ CE); 3.61-3.52 (m, 2H, CH₂ C3 carbon chain); 3.36-3.21 (m, 2H, CH₂ tetrazolium); 3.14 (s, 9H, 3CH₃); 2.74-2.65 (m, 2H, CH₂ CE); 2.27 (s, 1H, CH₃ tosylate); 2.16-2.04 (m, 2H, CH₂ C3 carbon chain); 1.43 (s, 9H, CH₃ tBu); 1.24-1.17 (t, J=10.0 Hz, 2H, CH₃ tetrazolium). ¹³C NMR (100 MHz, CDCl₃) σ_{c} (ppm) 167.89 (C═O); 163.90 (Cq tetrazolium); 156.11 (C═O); 143.71, 141.25 (Cq Fmoc); 143.38, 139.70 (Cq tosylate); 128.82, 125.76 (CH Ar tosylate); 127.81, 127.17, 125.12, 120.04 (CH Ar Fmoc); 117.13 (Cq CE); 83.39 (Cq tBu); 67.90 (CH₂); 67.22 (CH₂ Fmoc); 64.89, 63.34 (CH₂ C3 carbon chain); 62.71 (CH₂ CE); 54.95 (CH); 53.48 (CH₃); 47.22 (CH₂ tetrazolium); 47.05 (CH Fmoc); 27.97 (CH₃ tBu); 24.12 (CH₂ C3 carbon chain); 21.28 (CH₃ tosylate); 19.72 (CH₂ CE); 6.68 (CH₃ tetrazolium). ³¹P NMR (161 MHz, CDCl₃) σ_{P} (ppm) −2.19. HRMS (ESI⁺) m/z for calculated C₃₁H₄₃N₃O₈P⁺ [M]⁺ 616.2782; Found 616.2757. NMR spectra shown in FIGS. 11A-C.

Compound 6b. Compound 5 (1.77 g, 3.03 mmol, 1.0 equiv.) and compound 3b (1.21 g, 3.65 mmol, 1.2 equiv.) was first dried by co-evaporation with dry toluene and dry ACN. Dry ACN was added under argon and ETT (0.25 M in ACN) (12.1 mL, 3.03 mmol, 1.0 equiv.) was added slowly with a syringe. The mixture was stirred 2 h at room temperature. After reaching completion, the reaction mixture was quenched with methanol (257 μL). Meta-chloroperoxybenzoic acid (mCPBA) (1.05 g, 6.08 mmol, 2.0 equiv.) was added by portion to the mixture. After 30 min of stirring, the mixture was reduced under vacuum. The crude was then purified by column chromatography on silica gel using a gradient of MeOH in DCM (0-30%) as eluent, to afford 6b as a mixture of tetrazolium (major counter anion) and tosylate salts (1.72 g, 2.18 mmol, yield 72%).

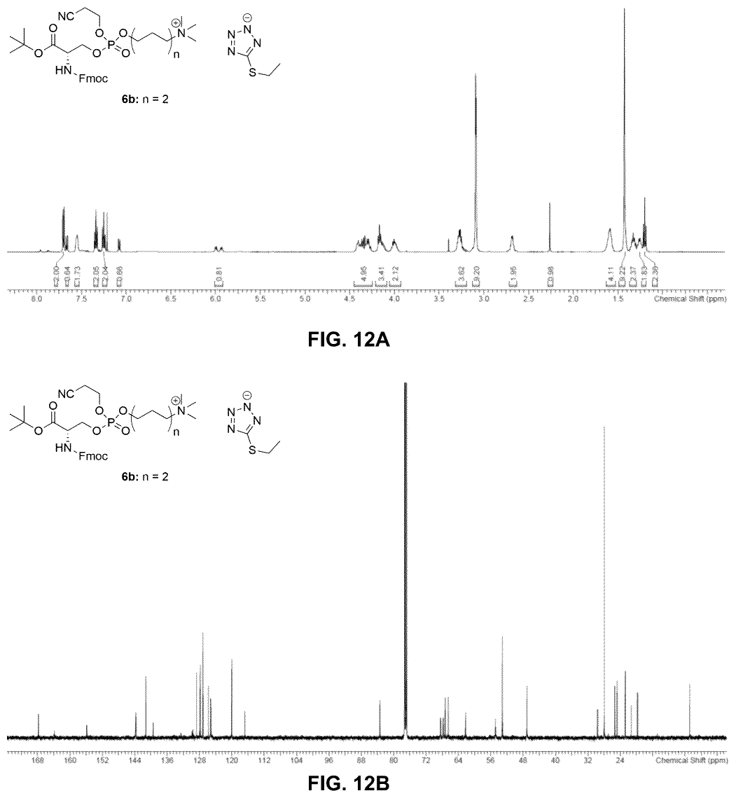

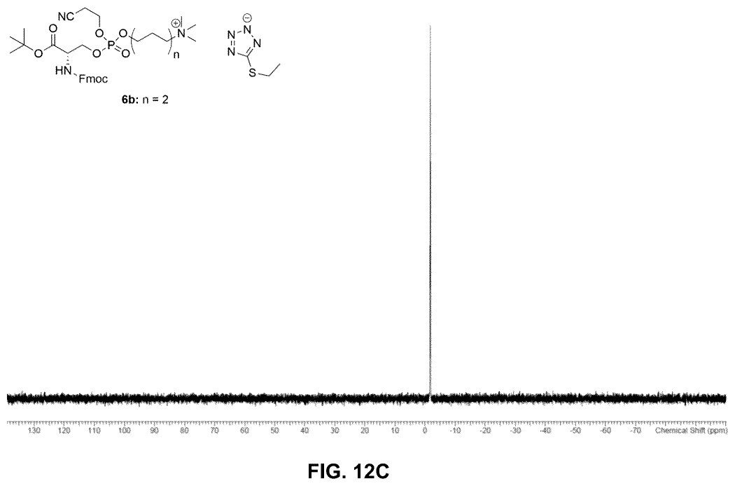

¹H NMR (500 MHz, CDCl₃) σ_{H} (ppm) 7.70 (d, J=10.0 Hz, 2H, Ar Fmoc); 7.66 (d, J=10.0 Hz, 1H, Ar tosylate); 7.55 (m, 2H, Ar Fmoc); 7.34 (t, J=10.0 Hz, 2H, Ar Fmoc); 7.25 (t, J=10.0 Hz, 2H, Ar Fmoc); 7.08 (d, J=10.0 Hz, 1H, Ar tosylate); 6.01-5.92 (dd, J=30.0 Hz, J=5.0 Hz, 1H, NH); 4.45-4.25 (m, 5H, 2*CH Fmoc+CH₂+CH); 4.22-4.08 (m, 3H, CH Fmoc+CH₂ CE); 4.06-3.93 (m, 2H, CH₂ C6 carbon chain); 3.32-3.19 (m, 3H, CH₂ C6 carbon chain+CH₂ tetrazolium); 3.09 (d, J=3.36 Hz, 9H, 3CH₃); 2.72-2.63 (m, 2H, CH₂ CE); 2.26 (s, 1H, CH₃ tosylate); 1.64-1.53 (m, 4H, 2*CH₂ C6 carbon chain); 1.43 (s, 9H, CH₃ tBu); 1.37-1.29 (m, 2H, CH₂ C6 carbon chain); 1.29-1.23 (m, 2H, CH₂ C6 carbon chain); 1.23-1.17 (t, J=10.0 Hz, 2H, CH₃ tetrazolium). ¹³C NMR (100 MHz, CDCl₃) σ_{c} (ppm) 167.81 (C═O); 163.92 (Cq tetrazolium); 155.90, 155.85 (C═O); 143.78, 141.74, 143.72, 143.69 (Cq Fmoc); 141.26, 139.46 (Cq tosylate); 128.73, 125.78 (CH Ar tosylate); 127.81, 127.16, 125.21, 125.16, 120.04 (CH Ar Fmoc); 116.84, 116.82 (Cq CE); 83.42 (Cq tBu); 68.45 (CH₂ C6 carbon chain); 67.78 (CH₂); 67.28 (CH₂ Fmoc); 66.49 (CH₂ C6 carbon chain); 62.22 (CH₂ CE); 54.87 (CH); 53.17 (CH₃); 47.10 (CH₂ tetrazolium); 47.04 (CH Fmoc); 29.65, 29.60 (CH₂ C6 carbon chain); 27.96 (CH₃ tBu); 25.37, 24.78, 22.76 (CH₂ C6 carbon chain); 21.28 (CH₃ tosylate); 19.76, 19.70 (CH₂ CE); 6.85 (CH₃ tetrazolium). ³¹P NMR (161 MHz, CDCl₃) σ_{P} (ppm) −1.69, −1.72. HRMS (ESI⁺) m/z for calculated C₃₄H₄₉N₃O₈P⁺ [M]⁺ 658.3252; Found 658.3227. NMR spectra shown in FIGS. 12A-C.

Compound 6c. Compound 5 (1.69 g, 2.90 mmol, 1.0 equiv.) and compound 3c (1.30 g, 3.48 mmol, 1.2 equiv.) was first dried by co-evaporation with dry toluene and dry ACN. Dry ACN was added under argon and ETT (0.25 M in ACN) (11.6 mL, 2.90 mmol, 1.0 equiv.) was added slowly with a syringe. The mixture was stirred 2 h at room temperature. After reaching completion, the reaction mixture was quenched with methanol (246 μL). Meta-chloroperoxybenzoic acid (mCPBA) (1.00 g, 5.79 mmol, 2.0 equiv.) was added by portion to the mixture. After 30 min of stirring, the mixture was reduced under vacuum. The crude was then purified by column chromatography on silica gel using a gradient of MeOH in DCM (0-30%) as eluent, to afford 6c as a mixture of tetrazolium (major counter anion) and tosylate salts (1.46 g, 1.76 mmol, yield 61%).

¹H NMR (500 MHz, CDCl₃) σ_{H} (ppm) 7.70 (d, J=10.0 Hz, 2H, Ar Fmoc); 7.68 (d, J=10.0 Hz, 1H, Ar tosylate); 7.55 (m, 2H, Ar Fmoc); 7.34 (t, J=10.0 Hz, 2H, Ar Fmoc); 7.26 (t, J=10.0 Hz, 2H, Ar Fmoc); 7.08 (d, J=10.0 Hz, 1H, Ar tosylate); 5.92-5.81 (dd, J=15.0 Hz, J=10.0 Hz, 1H, NH); 4.47-4.23 (m, 5H, 2*CH Fmoc+CH₂+CH); 4.21-4.09 (m, 3H, CH Fmoc+CH₂ CE); 4.07-3.94 (m, 2H, CH₂ C9 carbon chain); 3.34-3.20 (m, 4H, CH₂ C9 carbon chain+CH₂ tetrazolium); 3.12 (d, J=2.44 Hz, 9H, 3CH₃); 2.73-2.61 (m, 2H, CH₂ CE); 2.27 (s, 1H, CH₃ tosylate); 1.66-1.52 (m, 4H, 2*CH₂ C9 carbon chain); 1.44 (d, J=1.22 Hz, 9H, CH₃ tBu); 1.34-1.15 (m, 13H, 5*CH₂ C9 carbon chain+CH₃ tetrazolium). ¹³C NMR (100 MHz, CDCl₃) σ_{c} (ppm) 167.77 (C═O); 163.93 (Cq tetrazolium); 155.83 (C═O); 143.79, 143.72, 143.69 (Cq Fmoc); 141.27, 139.40 (Cq tosylate); 128.71, 125.80 (CH Ar tosylate); 127.80, 127.13, 125.19, 125.16, 120.03 (CH Ar Fmoc); 116.56 (Cq CE); 83.42 (Cq tBu); 68.81 (CH₂ C9 carbon chain); 67.82 (CH₂); 67.36, 67.32 (CH₂ Fmoc); 66.84 (CH₂ C9 carbon chain); 62.08 (CH₂ CE); 54.83 (CH); 53.17 (CH₃); 47.14 (CH Fmoc); 47.05 (CH₂ tetrazolium); 30.04, 29.99, 28.97, 28.81, 28.70 (CH₂ C9 carbon chain); 27.96 (CH₃ tBu); 25.93, 25.15, 22.97 (CH₂ C9 carbon chain); 21.30 (CH₃ tosylate); 19.72, 19.67 (CH₂ CE); 6.82 (CH₃ tetrazolium). ³¹P NMR (161 MHz, CDCl₃) σ_{P} (ppm) −1.60, −1.65. HRMS (ESI⁺) m/z for calculated C₃₇H₅₅N₃O₈P⁺ [M]⁺700.3721;

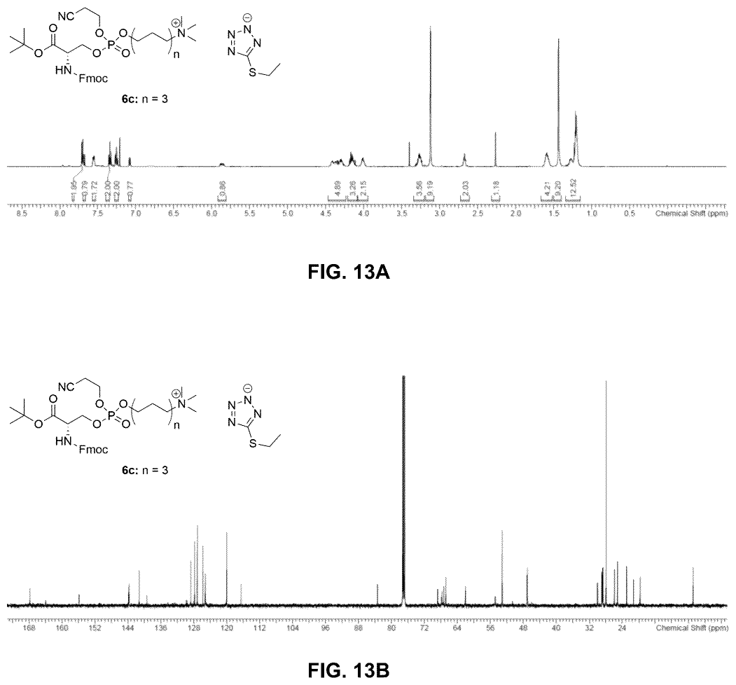

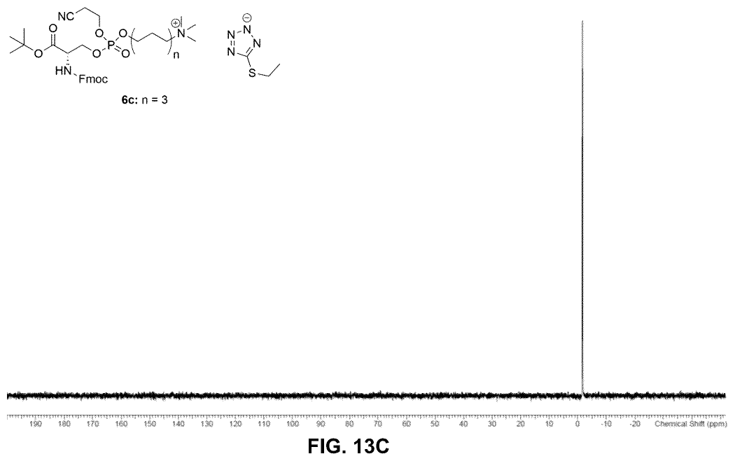

Found 700.3692. NMR spectra shown in FIGS. 13A-C.

Compound 7a. Compound 6a (4.40 g, 5.90 mmol, 1.0 equiv.) was dissolved in 101.8 mL of (1:1) solution of TFA: dry DCM. Triisopropylsilene (4.47 mL, 21.8 mmol, 3.7 equiv.) was added and the mixture was stirred at room temperature for 2 h. The solvent and TFA were evaporated and the residue was purified by reverse phase HPLC (C₁₈, Buffer A=Water, Buffer B=ACN, Gradient=5-65% of B in 12 min). The ACN was removed under vacuum and the aqueous solution was freeze-dried. The lyophilized powder was dissolved in 10% diisopropylamine (16.6 mL) in ACN (149.6 mL) and the mixture was stirred at room temperature for 2 h. The solvent was evaporated under vacuum and the crude was purified by reverse phase HPLC (C₁₈, Buffer A=Water, Buffer B=ACN, Gradient=5-65% of B in 12 min). The ACN was removed under vacuum and the aqueous solution was freeze-dried to afford 7a as diisopropylammonium salt (0.72 g, 1.18 mmol, yield 36%).

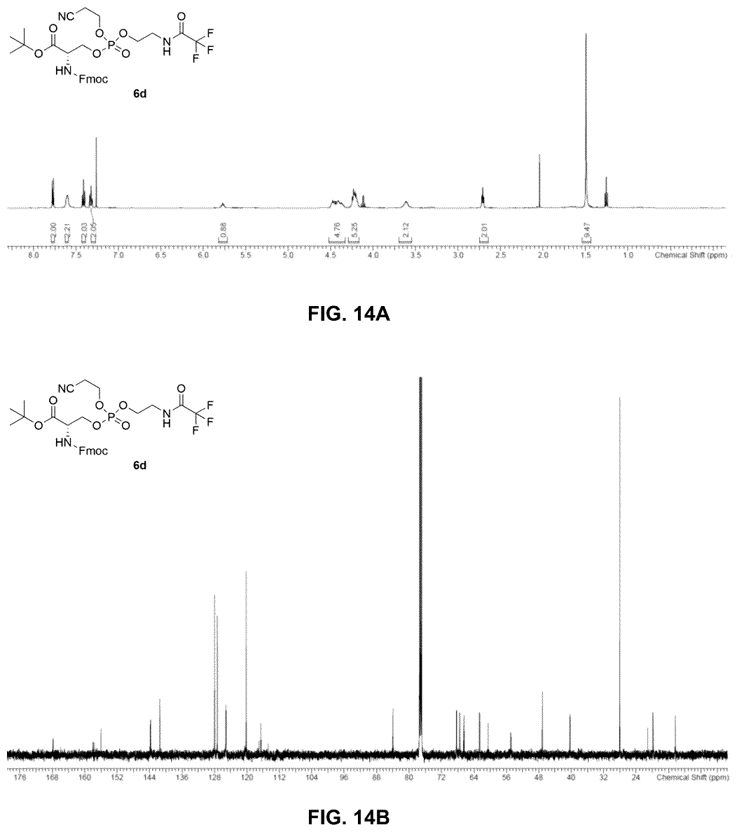

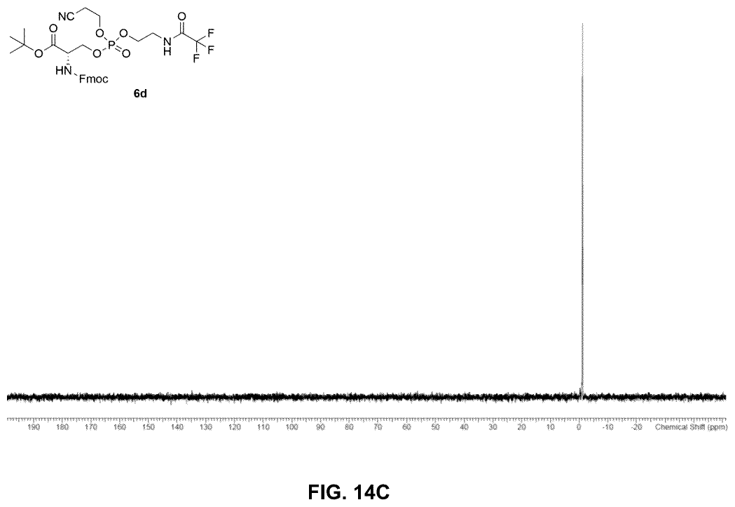

¹H NMR (500 MHz, MeOD) σ_{H} (ppm) 7.69 (t, J=10.0 Hz, 2H, Ar Fmoc); 7.64-7.57 (m, 2H, Ar Fmoc); 7.31-7.26 (m, 2H, Ar Fmoc); 7.26-7.18 (m, 2H, Ar Fmoc); 6.03 (s, 1H, NH); 4.29-4.22 (m, 1H, CH); 4.21-4.05 (m, 4H, 2*CH Fmoc+CH₂); 3.93-3.79 (m, 2H, CH₂ C3 carbon chain); 3.65-3.57 (m, 1H, CH Fmoc); 3.41-3.34 (m, 2H, CH₂ C3 carbon chain); 3.31-3.23 (m, 2H, 2CH diisopropylamine); 3.03 (d, J=15.0 Hz, 9H, 3CH₃); 2.05-1.90 (m, 2H, CH₂ C3 carbon chain); 1.17 (d, J=5.0 Hz, 12H, 4CH₃ diisopropylamine). ¹³C NMR (100 MHz, MeOD) ac (ppm) 174.75 (C═O); 156.91 (C═O); 144.30, 144.09, 143.73, 141.27, 140.17, 138.07 (Cq Fmoc); 128.63, 127.51, 126.95, 125.22, 125.09, 120.79, 119.62, 119.42 (CH Ar Fmoc); 66.75 (CH₂); 66.52 (CH); 64.62 (CH Fmoc); 64.24 (CH₂ C3 carbon chain); 62.12, 61.79 (CH₂ C3 carbon chain); 57.15, 57.08, 55.53, 55.47 (CH₂ Fmoc); 52.36 (CH₃); 46.81 (CH diisopropylamine); 24.22, 24.16 (CH₂ C3 carbon chain); 18.57 (CH₃ diisopropylamine). ³¹P NMR (161 MHz, MeOD) σ_{P} (ppm) 0.17. HRMS (ESI⁻) m/z for calculated C₂₄H₃₀N₂O₈P⁻ [M]⁻ 505.1745; Found 505.1737. NMR spectra shown in FIGS. 14A-C.

Compound 7b. Compound 6b (1.72 g, 2.18 mmol, 1.0 equiv.) was dissolved in 36.4 mL of (1:1) solution of TFA: dry DCM. Triisopropylsilene (1.65 mL, 8.08 mmol, 3.7 equiv.) was added and the mixture was stirred at room temperature for 2 h. The solvent and TFA were evaporated and the residue was purified by reverse phase HPLC (C₁₈, Buffer A=Water, Buffer B=ACN, Gradient=10-65% of B in 12 min). The ACN was removed under vacuum and the aqueous solution was freeze-dried. The lyophilized powder was dissolved in 10% diisopropylamine (5.88 mL) in ACN (52.8 mL) and the mixture was stirred at room temperature for 2 h. The solvent was evaporated under vacuum and the crude was purified by reverse phase HPLC (C₁₈, Buffer A=Water, Buffer B=ACN, Gradient=10-65% of B in 12 min). The ACN was removed under vacuum and the aqueous solution was freeze-dried to afford 7b as diisopropylammonium salt (0.20 g, 0.31 mmol, yield 27%).

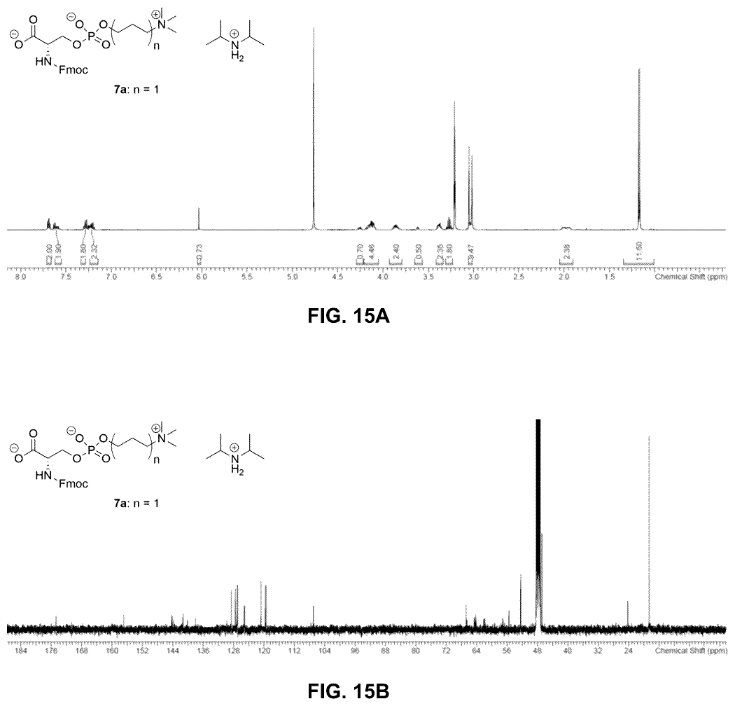

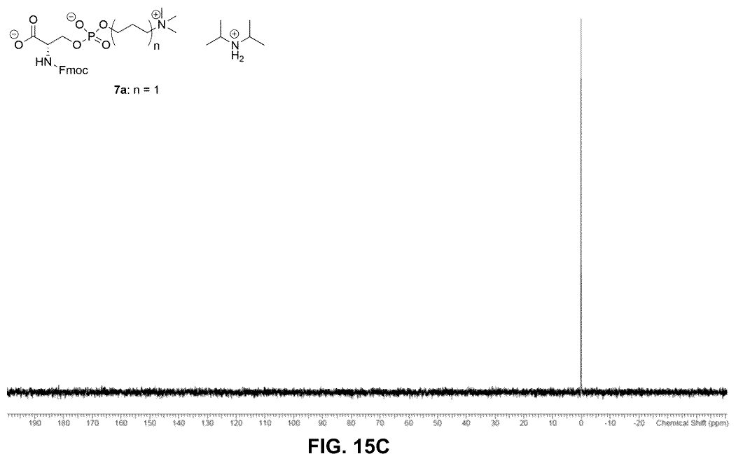

¹H NMR (500 MHz, DMSO) σ_{H} (ppm) 7.91-7.82 (m, 2H, Ar Fmoc); 7.72-7.68 (m, 2H, Ar Fmoc); 7.44-7.39 (m, 2H, Ar Fmoc); 7.37-7.30 (m, 2H, Ar Fmoc); 6.28 (s, 1H, NH); 4.27-4.13 (m, 3H, CH+CH₂); 4.01-3.78 (m, 2H, 2*CH Fmoc); 3.68-3.55 (m, 2H, CH₂ C6 carbon chain); 3.28-3.24 (m, 3H, CH Fmoc+CH₂ C6 carbon chain); 3.08-3.00 (m, 11H, 3CH₃+2CH diisopropylamine); 1.72-1.60 (m, 2H, C6 carbon chain); 1.55-1.43 (m, 2H, C6 carbon chain); 1.38-1.22 (m, 4H, 2*CH₂ C6 carbon chain); 1.18-1.01 (m, 12H, 4CH₃ diisopropylamine). ¹³C NMR (100 MHz, CDCl₃) c, (ppm) 171.31 (C═O); 155.32 (C═O); 144.00, 140.66, 139.41, 137.42, 128.94 (Cq Fmoc); 127.60, 127.30, 127.13, 125.32, 125.27, 121.40, 120.08, 120.04 (CH Ar Fmoc); 65.52, 65.15 (CH+CH Fmoc); 63.50 (CH₂ C6 carbon chain); 57.34 (CH Fmoc); 55.96 (CH₂ C6 carbon chain); 52.07 (CH₃); 46.70 (CH₂); 45.07 (CH diisopropylamine); 29.86, 29.81 (CH₂ C6 carbon chain); 25.26, 24.78 (CH₂ C6 carbon chain); 21.86 (CH₂ C6 carbon chain); 20.46 (CH₃ diisopropylamine). ³¹P NMR (161 MHz, DMSO) σ_{P} (ppm) 1.64, 1.57, −0.05, −0.23. HRMS (ESI⁻) m/z for calculated C₂₇H₃₆N₂O₈P⁻ [M]⁻ 547.2215; Found 547.2191. NMR spectra shown in FIGS. 15A-C.

Compound 7c. Compound 6c (1.46 g, 1.76 mmol, 1.0 equiv.) was dissolved in 29.4 mL of (1:1) solution of TFA: dry DCM. Triisopropylsilene (1.33 mL, 6.51 mmol, 3.7 equiv.) was added and the mixture was stirred at room temperature for 2 h. The solvent and TFA were evaporated and the residue was purified by reverse phase HPLC (C₁₈, Buffer A=Water, Buffer B=ACN, Gradient=15-70% of B in 12 min). The ACN was removed under vacuum and the aqueous solution was freeze-dried. The lyophilized powder was dissolved in 10% diisopropylamine (4.88 mL) in ACN (43.9 mL) and the mixture was stirred at room temperature for 2 h. The solvent was evaporated under vacuum and the crude was purified by reverse phase HPLC (C₁₈, Buffer A=Water, Buffer B=ACN, Gradient=15-70% of B in 12 min). The ACN was removed under vacuum and the aqueous solution was freeze-dried to afford 7c as diisopropylammonium salt (0.17 g, 0.25 mmol, yield 25%).

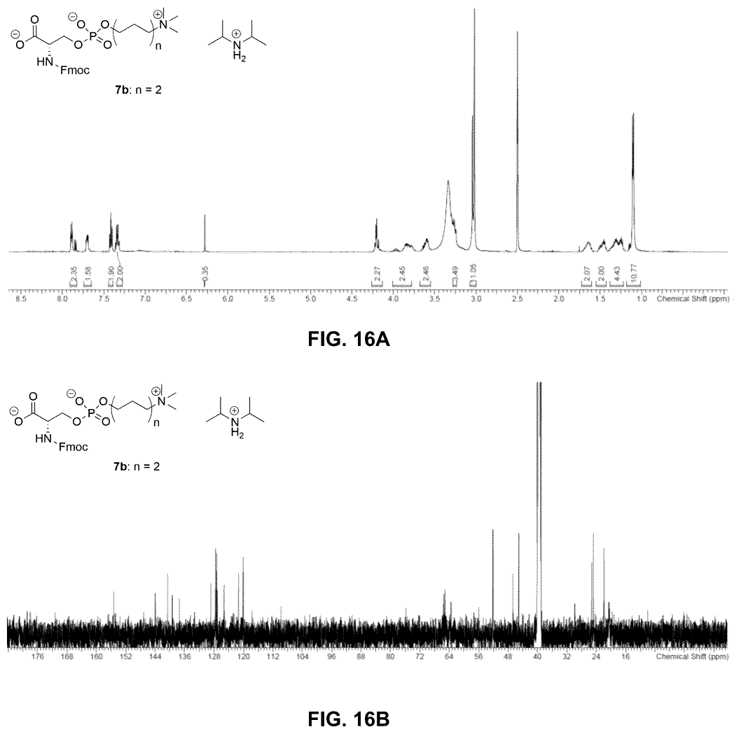

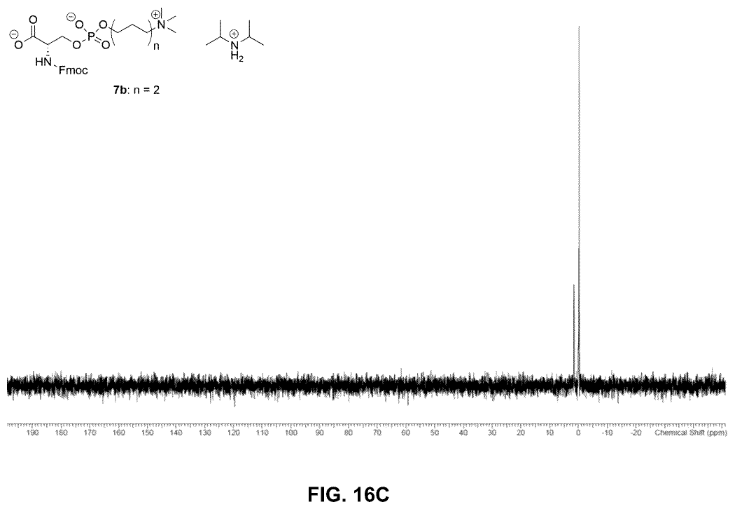

¹H NMR (500 MHz, DMSO) σ_{H} (ppm) 7.90-7.89, 7.85-7.83 (m, 2H, Ar Fmoc); 7.88-7.87, 7.72-7.68 (m, 2H, Ar Fmoc); 7.44-7.39 (m, 2H, Ar Fmoc); 7.37-7.30 (m, 2H, Ar Fmoc); 6.28 (s, 1H, NH); 4.01-3.90 (m, 1H, CH Fmoc); 3.86-3.74 (m, 2H, CH+CH Fmoc); 3.64-3.58 (m, 2H, CH₂ C9 carbon chain); 3.28-3.23 (m, 5H, CH₂+CH Fmoc+CH₂ C9 carbon chain); 3.04 (s, 9H, 3CH₃); 2.97-2.87 (m, 2H, 2CH diisopropylamine); 1.71-1.62 (m, 2H, C9 carbon chain); 1.49-1.43 (m, 2H, C9 carbon chain); 1.33-1.25 (m, 10H, 5*CH₂ C9 carbon chain); 1.01 (d, J=10.0 Hz, 12H, 4CH₃ diisopropylamine). ¹³C NMR (100 MHz, CDCl₃) σ_{c} (ppm) 174.27 (C═O); 156.88 (C═O); 139.41, 137.42 (Cq Fmoc); 128.94, 127.30, 121.40, 120.04 (CH Ar Fmoc); 65.19 (CH₂ C9 carbon chain); 63.92 (CH+CH Fmoc+CH₂ Fmoc+CH₂ C9 carbon chain); 56.19 (CH₂); 52.12 (CH₃); 44.78 (CH diisopropylamine); 30.24, 28.53, 28.27, 28.14, 25.62, 25.16 (CH₂ C9 carbon chain); 21.93 (CH₂ C9 carbon chain+CH₃ diisopropylamine). ³¹P NMR (161 MHz, DMSO) σ_{P} (ppm) 1.52, −0.08. HRMS (ESI⁻) m/z for calculated C₃₀H₄₂N₂O₈P⁻ [M]⁻ 589.2684; Found 589.2657. NMR spectra shown in FIGS. 16A-C.

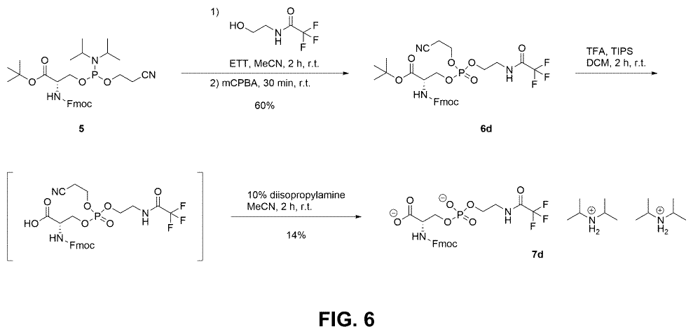

### 3. Synthesis of Ethanolamine Moiety 7d is Shown in FIG. 6.

Compound 6d. Compound 5 (3.41 g, 5.84 mmol, 1.0 equiv.) was first dried by co-evaporation with dry toluene and dry ACN. Dry ACN was added under argon and N-(2-hydroxyethyl)trifluoroacetamide (3.25 mL, 29.2 mmol, 5.0 equiv.) and ETT (0.25 M in ACN) (23.4 mL, 5.84 mmol, 1.0 equiv.) was added slowly with a syringe. The mixture was stirred 2 h at room temperature. After reaching completion, the reaction mixture was quenched with methanol (470 μL). Meta-chloroperoxybenzoic acid (mCPBA) (2.02 g, 11.7 mmol, 2.0 equiv.) was added by portion to the mixture. After 30 min of stirring, the mixture was reduced under vacuum. The crude was then purified by column chromatography on silica gel using a gradient of AcOEt in Hexane (40-90%) as eluent, to afford 6d as a viscous oil (2.30 g, 3.51 mmol, yield 60%).

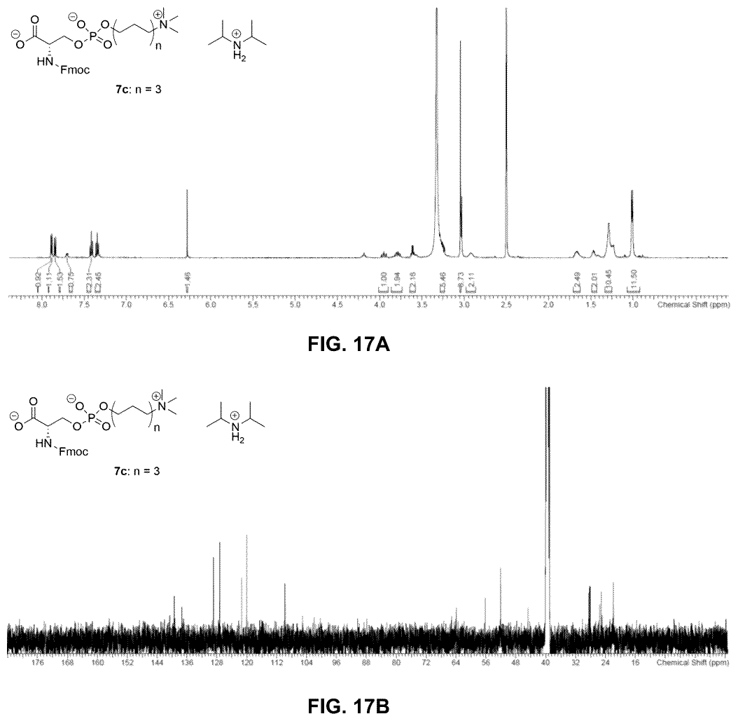

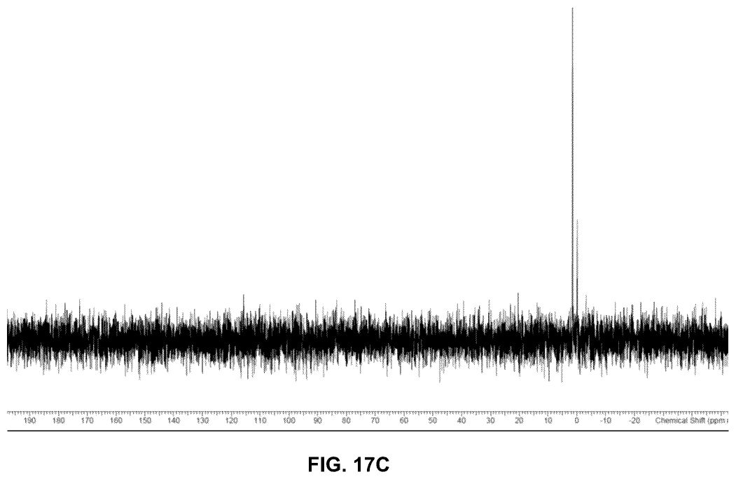

¹H NMR (500 MHz, CDCl₃) σ_{H} (ppm) 7.77 (d, J=10.0 Hz, 2H, Ar Fmoc); 7.62-7.59 (m, 2H, Ar Fmoc); 7.40 (t, J=10.0 Hz, 2H, Ar Fmoc); 7.35-7.30 (m, 2H, Ar Fmoc); 5.82-5.72 (m, 1H, NH Fmoc); 4.53-4.33 (m, 5H, CH₂ Fmoc+CH₂+CH); 4.29-4.16 (m, 5H, CH Fmoc, CH₂ CE, CH₂ ethanolamine carbon chain); 3.70-3.55 (m, 2H, CH₂ ethanolamine carbon chain); 2.75-2.64 (m, 2H, CH₂ CE); 1.50 (s, 9H, CH₃ tBu); ¹³C NMR (100 MHz, CDCl₃) σ_{c} (ppm) 167.80 (C═O); 157.97, 157.67 (C═O); 156.00 (C═O); 143.85, 143.72, 141.47, 141.43 (Cq Fmoc); 127.97, 127.26, 125.22, 125.13, 120.21 (CH Ar Fmoc); 116.58 (Cq CE); 114.76 (CF₃); 84.00 (Cq tBu); 68.28 (CH₂); 67.48 (CH); 66.47 (CH₂ ethanolamine carbon chain); 62.66, 62.62, 62.59, 62.55 (CH₂ CE); 54.93 (CH₂ Fmoc); 47.14 (CH Fmoc); 40.35, 40.31 (CH₂ ethanolamine carbon chain); 28.05 (CH₃ tBu); 19.87, 19.84, 19.81, 19.79 (CH₂ CE). ³¹P NMR (161 MHz, CDCl₃) σ_{P} (ppm) −1.14, −1.18. HRMS (ESI⁺) m/z for calculated C₂₉H₃₃F₃N₃O₉P [M+H]⁺ 656.1979; Found 656.1960. NMR spectra shown in FIGS. 17A-C.

Compound 7d. Compound 6d (2.30 g, 3.51 mmol, 1.0 equiv.) was dissolved in 58.5 mL of (1:1) solution of TFA: dry DCM. Triisopropylsilene (2.66 mL, 13.0 mmol, 3.7 equiv.) was added and the mixture was stirred at room temperature for 2 h. The solvent and TFA were evaporated and the residue was purified by column chromatography on silica gel using a gradient of MeOH in DCM (0-30%) as eluent, to afford the crude compound. The crude compound was dissolved in 10% diisopropylamine (3.14 mL) in ACN (95.7 mL) and the mixture was stirred at room temperature for 2 h. The solvent was evaporated under vacuum and the crude was purified by column chromatography on silica gel using a gradient of MeOH in DCM (0-30%) as eluent, to afford 7d as a white solid (0.38 g, 0.51 mmol, yield 14%) (FIG. 6)

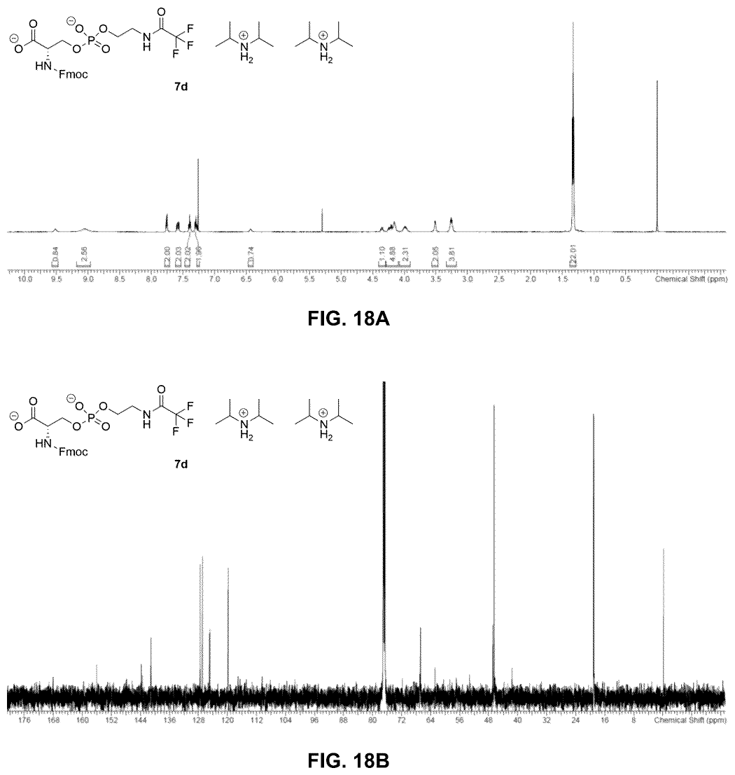

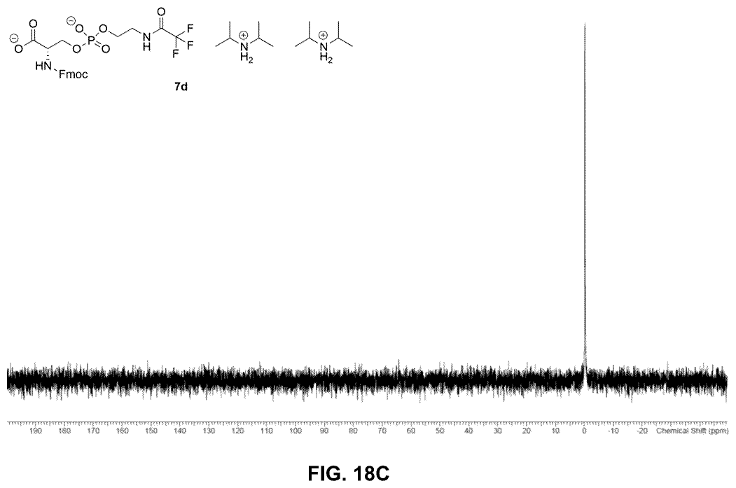

¹H NMR (500 MHz, CDCl₃) σ_{H} (ppm) 9.52 (s, 1H, NH trifluoroacetyl group), 9.18-8.96 (m, 3H, NH₂⊕ diisopropylamine); 7.76 (d, J=10.0 Hz, 2H, Ar Fmoc); 7.62-7.53 (m, 2H, Ar Fmoc); 7.40 (t, J=10.0 Hz, 2H, Ar Fmoc); 7.30 (t, J=10.0 Hz, 2H, Ar Fmoc); 6.43 (s, 1H, NH Fmoc); 4.41-4.30 (m, 1H, CH); 4.28-4.09 (m, 5H, CH Fmoc+CH₂ Fmoc+CH₂); 4.07-3.91 (m, 2H, CH₂ ethanolamine carbon chain); 3.56-3.46 (m, 2H, CH₂ ethanolamine carbon chain); 3.34-3.18 (m, 4H, 2×2CH diisopropylamine); 1.33 (t, J=6.18 Hz, 22H, 8CH₃ diisopropylamine). ¹³C NMR (100 MHz, CDCl₃) σ_{c} (ppm) 168.05 (C═O); 159.70 (C═O); 156.09 (C═O); 143.83, 143.51, 141.13, 141.09 (Cq Fmoc); 127.61, 126.93, 124.98, 124.87, 119.85 (CH Ar Fmoc); 110.46 (CF₃); 66.99 (CH₂); 66.82 (CH); 62.87, 62.82 (CH₂ ethanolamine carbon chain); 56.93 (CH Fmoc); 46.91 (CH₂ Fmoc); 46.55 (CH diisopropylamine); 41.58 (CH₂ ethanolamine carbon chain); 19.13, 19.01 (CH₃ diisopropylamine). ³¹P NMR (161 MHz, CDCl₃) σ_{P} (ppm) −0.20. HRMS (ESI⁻) m/z for calculated C₂₂H₂₀F₃N₂O₉P²⁻ [M+H]⁻ 545.0942; Found 545.0916. NMR spectra shown in FIGS. 18A-C.

4. Synthesis of Lipid Functionalized Solid Support for the Preparation of Conjugated siRNAs

Lipid moieties were directly attached via a peptide bond to a controlled pore glass (CPG) functionalized by a C7 linker, as scheme S4 (FIG. 7).

Compound 7a and DCA-functionalized CPG 12a. First, the Fmoc group of functionalized CPG 8 (0.78 g, loading of 50.7 μmol/g, 39.5 μmol,) was removed using a solution of 20% piperidine in DMF (7.8 mL) for 15 minutes. This procedure was repeated twice to ensure complete deprotection of the Fmoc group. The amine-bearing CPG 9 was filtered off and washed successively with DMF (13 mL), DCM (13 mL), ACN (13 mL) and ether (13 mL), and dried under vacuum. Next, compound 7a (0.12 g, 0.20 mmol, 5.00 equiv.) was dissolved in DMF (11 mL). (Benzotriazol-1-yloxy)tris(dimethylamino)phosphoniumn hexafluorophosphate (BOP) (0.086 g, 0.20 mmol, 5.00 equiv.) and hydroxybenzotriazol (HOBt) (0.027 g, 0.20 mmol, 5.00 equiv.) were added and stirred until the solution went clear. 2,4,6-collidine (62.7 μL, 0.47 mmol, 12.00 equiv.) was added followed by CPG 9 (0.78 g, loading of 50.7 μmol/g, 39.5 μmol, 1.00 equiv.) and the suspension was mixed overnight on a rotary mixer. The CPG was filtered off and washed with DMF (12 mL), DCM (12 mL), ACN (12 mL) and ether (12 mL) and dried under vacuum. The CPG was capped with 10% N-methylimidazole in THF (CAP A) (6 mL) and acetic anhydride:pyridine:THF (1:2:2, v/v/v) (CAP B) (6 mL) for 1 h and was washed with DMF (12 mL), DCM (12 mL), ACN (12 mL) and ether (12 mL) and dried under vacuum.

Furthermore, CPG 10a (0.78 g, loading of 50.7 μmol/g, 39.5 μmol, 1.00 equiv.) was first treated with 20% piperidine in DMF (7.8 mL) for 15 minutes. This procedure was repeated twice to ensure complete deprotection of the Fmoc group. The amine-bearing CPG 11a was filtered off and washed successively with DMF (13 mL), DCM (13 mL), ACN (13 mL) and ether (13 mL), and dried under vacuum. Then, the CPG 11a was mixed with a mixture of DCA (0.081 g, 0.24 mmol, 6.0 equiv.), HATU (0.089 g, 0.23 mmol, 5.9 equiv.) and DIPEA (76.2 μL, 0.47 mmol, 11.8 equiv.) in DMF (5.5 mL). The suspension was mixed on a rotary mixer for 24 h. The CPG was then filtered off and washed with DMF (13 mL), DCM (13 mL), ACN (13 mL) and ether (13 mL), and dried under vacuum. The CPG was capped with 10% N-methylimidazole in THF (CAP A) (6 mL) and acetic anhydride:pyridine:THF (1:2:2, v/v/v) (CAP B) (6 mL) during 15 min and was washed with DMF (13 mL), DCM (13 mL), ACN (13 mL) and ether (13 mL) and dried under vacuum.

Compound 7b-d and DCA-functionalized CPG 12b-d. These CPGs 12b-d were synthesized using a protocol similar to CPG 12a above (See FIG. 7).

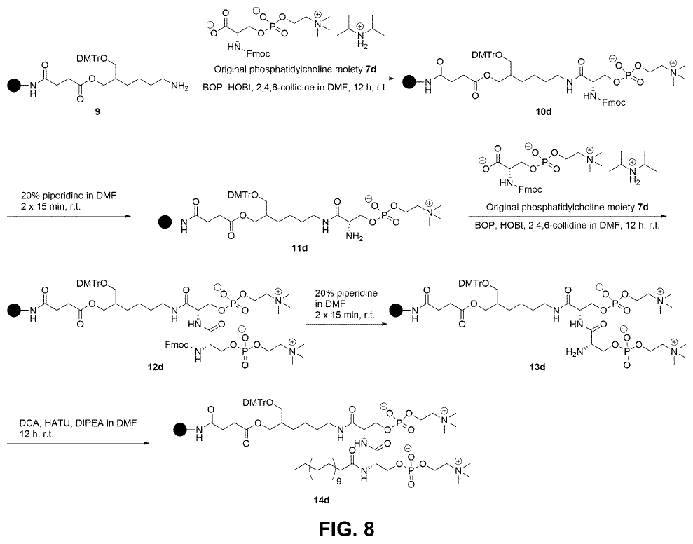

PC-PC-DCA immobilized CPG 14d. CPG 14d with two positive charge was synthesized by multi-coupling of original phosphatidylcholine moiety 7d as shown in FIG. 8.

### 5. Oligonucleotide Synthesis

Oligonucleotides were synthesized on an MerMaid-12 DNA/RNA synthesizer following standard solid phase oligonucleotide synthesis protocols. Oligonucleotides were synthesized at a 5-μmol scale using compound 7a-d and DCA-conjugated CPG 12a-d for the sense strand or at a 10-μmol scale using a Unylinker® terminus (ChemGenes, Wilmington, MA) for the antisense strand. Phosphoroamidites were prepared as 0.1 M solutions for 2′-O-methyl (ChemGenes, Wilmington, MA) and 2′-fluoro (BioAutomation, Irving, Texas) in ACN. 5-(Benzylthio)-1H-tetrazole (BTT) 0.25 M in ACN was used as coupling activator. Detritylations were performed using 3% dichloroacetic acid (DCA) in DCM for 80 s and capping was done with a 16% N-methylimidazole in THF (CAP A) and THF:acetic anhydride:2,6-lutidine, (80:10:10, v/v/v) (CAP B) for 15 s. Sulfurizations were carried out with 0.1 M solution of DDTT in ACN for 3 minutes. Oxidation was performed using 0.02 M iodine in THF:pyridine:water (70:20:10, v/v/v) for 80 s. Phosphoramidite coupling time was 250 s for all phosphonamidites.

### 6. Deprotection and Purification of Oligonucleotides

Sense strands were cleaved and deprotected using the solution of 50% NH₄OH and 50% MeNH₂ at room temperature for 2 h. Antisense strands were also cleaved and deprotected using the solution of conc. NH₄OH (3% DEA) at 45° C. for 16 h. The oligonucleotide solutions were then cooled in a freezer for a few minutes and dried under vacuum in a Speedvac. The resulting pellets were suspended in water. The final purification if oligonucleotides was performed on an Agilent Prostar System (Agilent, Santa Clara, CA). Sense strands were purified over a Hamilton HxSil C18 column (150×21.2) in a continuous gradient of sodium acetate: 90% Buffer A1 (50 mM sodium acetate in 5% acetonitrile), 10% Buffer B1 (acetonitrile) to 10% Buffer A1, 90% Buffer B1 at a flow rate of 10 mL/min for 18 min at 60° C. Antisense strands were purified over a Dionex NucleoPac PA-100 (9×250) in a continuous gradient of sodium perchlorate: 100% Buffer A2 (10 mM sodium perchlorate in 20% acetonitrile) to 20% Buffer A2, 80% Buffer B2 (1 M sodium perchlorate, 20% acetonitrile) at a flow rate of 10 ml/min for 30 min at 60° C. Purified oligonucleotides were collected, desalted by size-exclusion chromatography using a Sephadex G25 column (GE Healthcare Life Sciences, Marborough, MA) and lyophilized.

### 7. LC-MS Analysis of Oligonucleotides

The identify of oligonucleotides was established by LC-MS analysis on as Agilent 6530 accurate-mass Q-TOF LC/MS (Agilent technologies, Santa Clara, CA) using the following conditions: buffer A (9 mM triethylamine/100 mM hexafluoroisopropanol in water), buffer B (9 mM triethylamine/100 mM hexafluoroisopropanol in MeOH), column: Agilent advancebio oligonucleotides (Agilent technologies, Santa Clara, CA), 2.1×50 mm, gradient for sense strand: 0-2 min (1% B-40% B), 2-10.5 min (40% B-100% B), gradient for the antisense strand: 0-2 min (0% B-12% B), 2-10.5 min (12% B-30% B), 10.5-11 min (30% B-100% B).

TABLE 1
Sequence and observed mass of antisense and sense strands used in the
study.

| | | Calcd. | |
|---|---|---|---|
| | | Mass | Observed |
| Name | Sequence (5′→3′)^{a} | [M − H]⁻ | Mass |
| Antisense | V(mU)#(fU)#(mA)(fA)(fU)(fC)(mU)(fC)(mU)(fU)(mU)(fA) | 6615.4 | 6615.7 |
| (Htt) | (mC)#(fU)#(mG)#(fA)#(mU)#(mA)#(fU)#(mA) | | |
| Sense (Htt) | (mC)#(mA)#(fG)(mU)(fA)(mA)(fA)(mG)(fA)(mG)(mA)(mU) | 6184.6 | 6185.2 |
| Conjugate: | (fU)#(mA)#(mA)(dT)(dT)-DCA | | |
| DCA | | | |
| Sense (Htt) | (mC)#(mA)#(fG)(mU)(fA)(mA)(fA)(mG)(fA)(mG)(mA)(mU) | 6437.8 | 6437.3 |
| Conjugate: | (fU)#(mA)#(mA)(dT)(dT)-PC-DCA (C2) | | |
| PC-DCA (C2) | | | |
| Sense (Htt) | (mC)#(mA)#(fG)(mU)(fA)(mA)(fA)(mG)(fA)(mG)(mA)(mU) | 6451.9 | 6451.3 |
| Conjugate: | (fU)#(mA)#(mA)(dT)(dT)-PC-DCA (C3) | | |
| PC-DCA (C3) | | | |
| Sense (Htt) | (mC)#(mA)#(fG)(mU)(fA)(mA)(fA)(mG)(fA)(mG)(mA)(mU) | 6493.9 | 6493.4 |
| Conjugate: | (fU)#(mA)#(mA)(dT)(dT)-PC-DCA (C6) | | |
| PC-DCA (C6) | | | |
| Sense (Htt) | (mC)#(mA)#(fG)(mU)(fA)(mA)(fA)(mG)(fA)(mG)(mA)(mU) | 6536.0 | 6535.4 |
| Conjugate: | (fU)#(mA)#(mA)(dT)(dT)-PC-DCA (C9) | | |
| PC-DCA (C9) | | | |
| Sense (Htt) | (mC)#(mA)#(fG)(mU)(fA)(mA)(fA)(mG)(fA)(mG)(mA)(mU) | 6394.8 | 6395.3 |
| Conjugate: | (fU)#(mA)#(mA)(dT)(dT)-Ethanolamine-DCA | | |
| Ethanolamine- | | | |
| DCA | | | |
| Sense (Htt) | (mC)#(mA)#(fG)(mU)(fA)(mA)(fA)(mG)(fA)(mG)(mA)(mU) | 6691.1 | 6689.4 |
| Conjugate: | (fU)#(mA)#(mA)(dT)(dT)-PC-PC-DCA | | |
| PC-PC-DCA | | | |
| Antisense | V(mU)#(fU)#(mA)(fA)(fU)(fC)(mA)(fU)(mU)(fU)(mA)(fU) | 6952.6 | 6952.7 |
| (CD47) | (mG)(fU)#(mG)#(fA)#(mC)#(mU)#(mU)#(fU)#(mU) | | |
| Sense (CD47) | (mU)#(mC)#(mA)(fC)(mA)(fU)(mA)(fA)(mA)(fU)(mG)(mA) | 6425.8 | 6426.3 |
| Conjugate: | (mU)(fU)#(mA)#(mA)(dT)(dT)-DCA | | |
| DCA | | | |
| Sense (CD47) | (mU)#(mC)#(mA)(fC)(mA)(fU)(mA)(fA)(mA)(fU)(mG)(mA) | 6679.0 | 6678.3 |
| Conjugate: | (mU)(fU)#(mA)#(mA)(dT)(dT)-PC-DCA (C2) | | |
| PC-DCA (C2) | | | |
| Sense (CD47) | (mU)#(mC)#(mA)(fC)(mA)(fU)(mA)(fA)(mA)(fU)(mG)(mA) | 6693.0 | 6692.4 |
| Conjugate: | (mU)(fU)#(mA)#(mA)(dT)(dT)-PC-DCA (C3) | | |
| PC-DCA (C3) | | | |
| Sense (CD47) | (mU)#(mC)#(mA)(fC)(mA)(fU)(mA)(fA)(mA)(fU)(mG)(mA) | 6735.1 | 6734.4 |
| Conjugate: | (mU)(fU)#(mA)#(mA)(dT)(dT)-PC-DCA (C6) | | |
| PC-DCA (C6) | | | |
| Sense (CD47) | (mU)#(mC)#(mA)(fC)(mA)(fU)(mA)(fA)(mA)(fU)(mG)(mA) | 6777.2 | 6776.5 |
| Conjugate: | (mU)(fU)#(mA)#(mA)(dT)(dT)-PC-DCA (C9) | | |
| PC-DCA (C9) | | | |
| Sense (CD47) | (mU)#(mC)#(mA)(fC)(mA)(fU)(mA)(fA)(mA)(fU)(mG)(mA) | 6635.9 | 6636.3 |
| Conjugate: | (mU)(fU)#(mA)#(mA)(dT)(dT)-Ethanolamine-DCA | | |
| Ethanolamine- | | | |
| DCA | | | |
| Sense (CD47) | (mU)#(mC)#(mA)(fC)(mA)(fU)(mA)(fA)(mA)(fU)(mG)(mA) | 6932.2 | 6930.4 |
| Conjugate: | (mU)(fU)#(mA)#(mA)(dT)(dT)-PC-PC-DCA | | |
| PC-PC-DCA | | | |

^{a}V: 5′-E-VP; m: 2′-OMe; f: 2′-Fluoro; #: Phosphorothioate

### Animal Administrations:

All animal experiments were conducted in accordance with the guidelines of the University of Massachusetts Chan Medical school (UMass Chan) Institutional Animal Care and Use Committee (IACUC). All procedures were performed as approved under IACUC protocol #A-2411. Female wildtype FVB mice were purchased from The Jackson Laboratory (Bar Harbor, ME). Mice were 6-8 weeks old at the time of the experiments. siRNAs were formulated in phosphate buffered saline (PBS) and administered in a volume of 150 μL subcutaneously between the shoulder blades. The mice were housed at pathogen-free animal facilities at UMass Chan with a 12 h/12 h light/dark cycle, controlled temperature (23+/−1° C.), humidity (50% +/−20%) and ad libitum food and water.

### Peptide Nucleic Acid (PNA) Hybridization Assay:

Quantification of antisense strand accumulation in tissues was performed by the peptide nucleic acid (PNA) hybridization assay as previously described. Briefly, ˜0.2-7 mg of tissues were lysed in QuantiGene™ Homogenizing Solution (Invitrogen) containing 0.2 mg/mL Ambion™ Recombinant Proteinase K Solution (Invitrogen) using the TissueLyser™ II (Qiagen) and 3 mm Tungsten Carbide Beads (Qiagen) and incubated at 65° C. for half an hour. Following incubation, tissue lysates were treated with 3M potassium chloride to precipitate sodium dodecyl sulfate and pelleted by centrifugation at 4,000×g for 15 min. Supernatants were hybridized with Cy3-labelled PNA probes (PNABio, Thousand Oaks, CA, USA) fully complementary to the siRNA^{cd47} antisense strand and analyzed by HPLC (Agilent, Santa Clara, CA, USA) over a DNAPac PA100 anion-exchange column (Thermo Fisher Scientific). Cy3 fluorescence was monitored, and peaks integrated using a custom python script. Final concentrations of siRNA^{cd47} in tissue lysate were determined using a standard curve generated from samples containing known concentrations of siRNA^{cd47}.

QUANTIGENE Singleplex 2.0 (bDNA) Assay

Tissue lysates prepared in the same manner as above (see PNA hybridization assay) were used to measure htt and cd47 mRNA levels using the QuantiGene Singleplex assay kit (Thermo Fisher Scientific, Waltham, MA) with the following probe sets: mouse Htt (Huntingtin gene) (SB-14150 (Affymetrix)), mouse cd47 (SB-3029698 (Affymetrix)) and mouse Hprt (hypoxanthine-guanine phosphoribosyl transferase) (SB-15463 (Affymetrix)), according to the manufacturer's instructions.

### Example 1: Headgroup Variants Alter the Hydrophobicity of DCA-siRNA

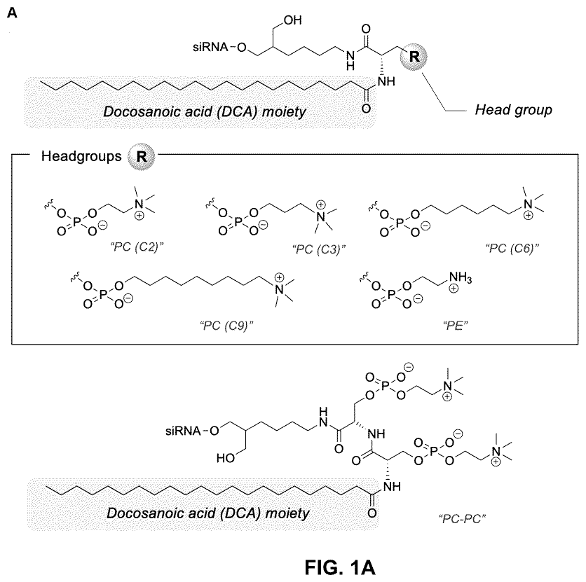

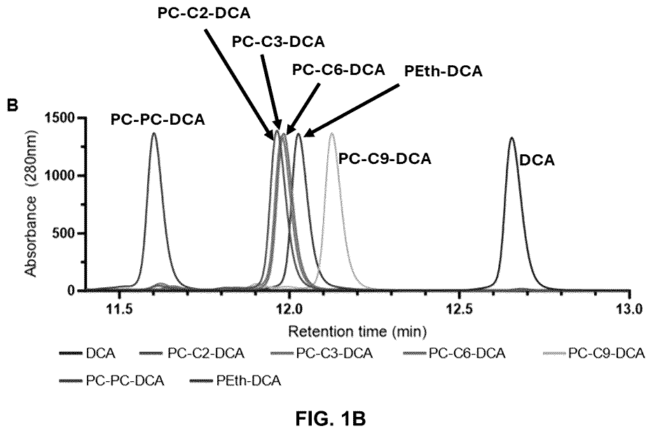

To evaluate the impact of chemical structural modulation of the headgroup on the properties of lipid conjugated siRNA, we synthesized DCA conjugated siRNA targeting mouse cd47 (siRNA^{cd47}) or mouse huntingtin (siRNA^{htt}) that have been previously published and characterized. We also synthesized conjugates functionalized with a single PC headgroup (PC-DCA) as previously reported (3, 14). Novel PC-DCA variants were synthesized with either ethyl (PC-C2-DCA), propyl (PC-C3-DCA), hexyl (PC-C6-DCA) or nonyl (PC-C9-DCA) bridge groups between the phosphate and ammonium moieties, named C2, C3, C6 and C9 respectively (FIG. 1A, and see Supplemental information). We also synthesized PC-PC-DCA, which is a single DCA functionalized with double PC headgroups, each with a C2 bridge group between the phosphate and the ammonium moiety. The final variant synthesized was DCA functionalized with phosphoethanolamine (PEth) which replaces the charged trimethylammonium group of PC with a non-substituted amino group connecting to the phosphate group by C2 bridge (FIG. 1A).

After synthesis, we measured the hydrophobicity of the passenger strands of siRNAhtt variants using reverse phase HPLC as previously published. In accordance with previous reports, addition of PC-C2 headgroup reduced the hydrophobicity compared to non-functionalized DCA variant. Increasing the bridge length from C2 to C3 or C6 did not significantly affect the hydrophobicity. However, C9 was clearly more hydrophobic than C2, C3 and C6 variants as evidenced by the shift in retention time on the reverse phase column. PEth was less hydrophobic than C9 and only slightly more hydrophobic than C2, C3 and C6. As expected, PC-PC was the least hydrophobic of all variants tested due to the presence of two PC headgroups (FIG. 1B).

### Example 2: Increasing Bridge Length Enhances Extrahepatic Tissue Accumulation

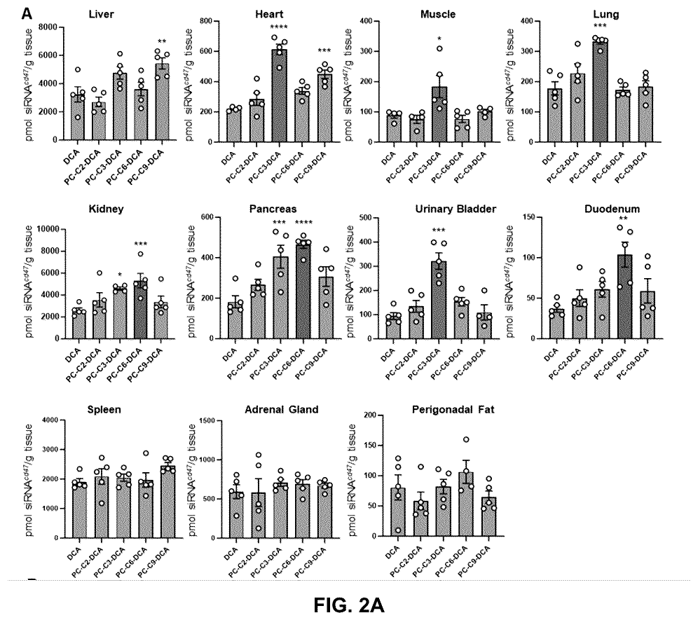

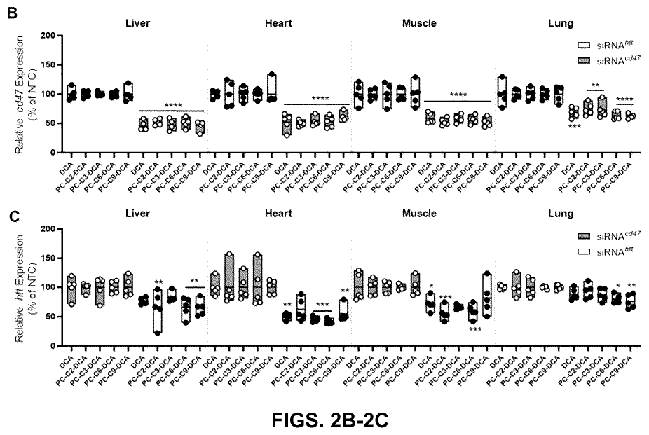

The panel of variants was tested in vivo in wildtype FVB mice. Briefly, mice were injected subcutaneously (intrascapular) with 20 mg/kg of siRNA variants and were sacrificed two weeks post injection. Eleven tissues were collected and the amount of siRNA^{cd47} in these tissues measured using the PNA assay. In almost all tissues tested, the addition of the PC headgroup significantly increased siRNA^{cd47} accumulation compared to the DCA conjugate (FIG. 2A). Increased accumulation was mediated by the bridge length with C3 variants showing higher accumulation that DCA in the heart (613.2 pmol/g vs 220.2 pmol/g, p<0.0001), muscle (183.5 pmol/g vs 88.02 pmol/g, p<0.05), lung (331.7 pmol/g vs 177.6 pmol/g, p<0.001) and urinary bladder (321 pmol/g vs 94 pmol/g, p<0.001). The C6 variant performed better than DCA in the kidney (5308 pmol/g vs 2605 pmol/g, p<0.001), pancreas (465.5 pmol/g vs 181.9 pmol/g, p<0.0001) and duodenum (103.7 pmol/g vs 36.9 pmol/g, p<0.01). The addition of the PC headgroup and changing the length of the bridge between the phosphate group and ammonium group did not affect siRNA^{cd47} accumulation in spleen, adrenal gland or fat.

The expression levels of cd47 and htt mRNA was measured in a subset of these tissues. All variants effectively and equally silenced cd47 gene expression in the liver, heart, muscle and lung with no significant differences between the linker variants tested (FIG. 2B). The silencing of htt gene expression was improved in certain variants, although these differences were not statistically significant (FIG. 2C). Specifically, in the liver, PC-C2-DCA (66% expression) was most efficacious and better than DCA control (76% expression). In the heart, C3 (46% expression) and C6 (42% expression) performed better than DCA (50% expression) and C2 (62% expression). In the muscle, C2 (55% expression) and C6 (59% expression) performed better than DCA (72% expression) and in the lung, C9 (76% silencing) was the best performing variant compared to DCA (88% expression) or C2 (93% expression).

### Example 3: Increasing the Valency of Linker Provides Tissue Specific Extrahepatic Accumulation

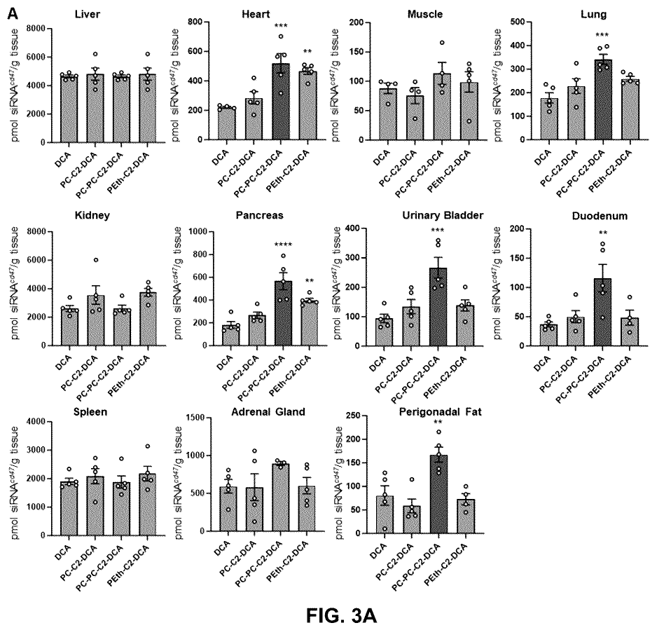

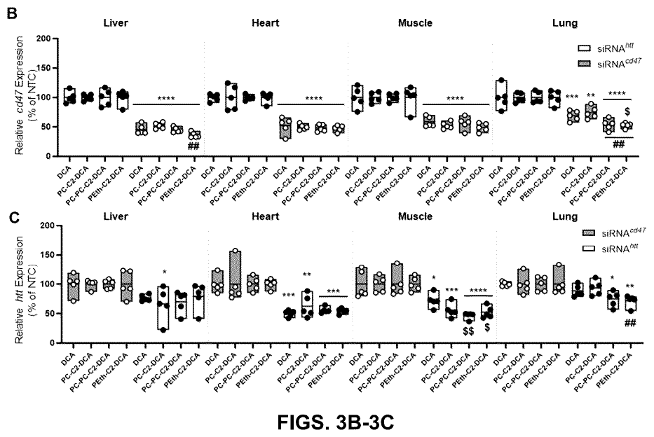

The effect of modifying the conjugate headgroup, either by increasing the valency of PC from one to two, or by changing the PC to PEth was also studied (FIG. 3A). The phosphatidylethanolamine head group is a primary alkyl amine and is commonly found in plasma membrane phospholipids. The pKa of phosphatidylethanolamine is known to be ˜8.5 suggesting that the amine exists mostly in its protonated charged form. This feature can provide insight about the impact of replacing PC with PEth in the lipid conjugate. The effect of the quaternary amine was also studied by increasing the valency of the headgroup using a PC-PC variant, as such variants differing in PC:lipid ratios may be found in nature and are relatively unexplored. The introduction of the double PC group did not affect siRNA^{cd47} accumulation in the liver, muscle, kidney, spleen, and adrenal gland. Interestingly significantly higher levels of siRNA^{cd47} was found in the PC-PC variant compared to the DCA variant in heart (519.8 pmol/g vs 220.2 pmol/g, p<0.001), lung (341.4 pmol/g vs 177.6 pmol/g, p<0.001), pancreas (565.9 pmol/g vs 181.9 pmol/g, p<0.0001), urinary bladder (266.5 pmol/g vs 94 pmol/g, p<0.001), duodenum (116.1 pmol/g vs 36.9 pmol/g, p<0.01) and perigonadal fat (167.2 pmol/g vs 80.5 pmol/g, p<0.01).

Similar to the linker variants, cd47 mRNA silencing was potent and similar across all headgroup variants compared to NTC treated animals in the liver, heart and muscle with no statistically significant differences between groups in heart and muscle (FIG. 3B). However, in the liver, PEth performed significantly better than PC-C2-DCA (36% expression vs 52% expression, p<0.01) and in the lung, PC-PC (54% expression, p<0.01) and PEth (53% expression, p<0.01) variants were significantly better than PC-C2-DCA (74% expression) in silencing cd47 mRNA. There was also significantly improved silencing for the PEth conjugate in the lung compared to the DCA conjugated siRNA (53% expression vs 69% expression, p<0.05). Relative htt mRNA expression was minimally affected in the liver with only PC-C2-DCA showing statistically significant silencing compared to NTC. Potent and significant silencing of htt mRNA was observed in the heart, muscle, and lung (FIG. 3C). In the muscle, PC-PC (47% expression, p<0.01) and PEth (52% expression, p<0.05) headgroups performed significantly better than DCA (72% expression). Silencing in the lung was also significantly improved with PEth compared to PC-C2-DCA (72% vs 93% expression, p<0.01).

### Discussion

The evolution of oligonucleotides as a clinically viable class of therapeutics has been preceded by incremental discoveries and innovations in chemistry that support the stability, safety, and tissue distribution of these molecules. In the field of siRNA therapeutics, the most clinically advanced delivery platform is the GalNAc conjugate, which provides receptor targeted delivery to liver hepatocytes and unprecedented levels and duration of silencing in humans (17-21). However, receptor mediated delivery targeted to extrahepatic organs is yet to be discovered. This lacuna in the siRNA toolbox is partially compensated for by the development of hydrophobic conjugates which while still delivering siRNAs primarily to the liver, allow for potentially therapeutic doses to accumulate in extrahepatic tissues like the placenta, skeletal and cardiac muscles after systemic administration and the skin, central nervous system, eye and lung after local administration (13, 14, 22-24).

The present disclosure and examples above further expand the hydrophobic conjugate toolbox by varying headgroup properties of PC-DCA conjugated siRNA. We chose PC-DCA conjugated siRNA to explore the relationship between headgroup properties and extrahepatic distribution as this is currently the most clinically advanced hydrophobic conjugate with phase I trials ongoing for the treatment of preeclampsia (16). As illustrated in the examples above, the extrahepatic distribution properties of DCA and PC-DCA can be improved by varying the size of the linker between the negatively charged phosphate and the positively charged choline moiety from C2 (conventional) to C3, C6 or C9. In particular, the data show that this increase in linker length can significantly enhance the accumulation of siRNA in the heart (˜2.8 fold vs DCA), muscle (˜2.1 fold vs DCA), lung (˜1.9 fold vs DCA), kidney (˜2 fold vs DCA), pancreas (˜2.6 fold vs DCA), urinary bladder (˜3.4 fold vs DCA) and duodenum (˜2.8 fold vs DCA) (FIG. 2A). The although the C9 linker did not enhance accumulation in any of the tissues in the mouse model, the C3 linker provided optimal heart, muscle, lung and urinary bladder accumulation and the C6 linker provided optimal kidney, pancreas and duodenum accumulation. Modulation of the valency (PC-PC-DCA (with two PC groups)) and type of headgroup (PEth-DCA (replacement of choline with ethanolamine)), also improved accumulation compared to the conventional PC-C2-DCA, with exceptional results for perigonadal fat (PC-PC-DCA was the best conjugate (˜2.1 fold vs DCA)) (FIG. 3A). Without being bound by theory, the molecular mechanism underlying the empirical evidence for the role of phosphate-choline bridge length in tissue accumulation may be due to the increased distance between the negatively charged phosphate group and the positively charged choline group in the net neutral PC headgroup. This charge separation may allow for local charge-based interactions with the extracellular matrix at each ionic pole and thereby increase residence time of the siRNA at the tissue site. The large differences in tissue accumulation among very minor structural variants such as between C2 and C3 PC-DCA could also be due to a difference in metabolic stability against endogenous phospholipases that are known to cleave natural PC-modified phospholipids (25).

Overall, these data provide evidence that chemical engineering of hydrophobically-modified oligonucleotides is critical for advancing the RNA therapeutic field and proof of principle that linker engineering is a viable path for improving extra-hepatic delivery and targeted silencing. The systematic modulation the DCA-conjugated oligonucleotide above illustrates this novel strategy for altering conjugate structure: modulation of the hydrophobic-conjugate linker moiety to enhance extrahepatic delivery.

Further embodiments can include replacing the charged choline group with a neutral butyl group and/or replacing the DCA with other hydrophobic conjugates that are in use like palmitic acid (C16) and myristic acid (C14), and adjusting the dose (e.g., >20 mg/kg), which can influence the tissue concentrations of siRNA and/or target silencing in extra-hepatic tissues.

### REFERENCES

1. A. Khvorova, J. K. Watts, The chemical evolution of oligonucleotide therapies of clinical utility. Nat Biotechnol 35, 238-248 (2017).
2. A. L. Jackson et al., Position-specific chemical modification of siRNAs reduces “off-target” transcript silencing. RNA 12, 1197-1205 (2006).
3. A. Biscans et al., Diverse lipid conjugates for functional extra-hepatic siRNA delivery in vivo. Nucleic Acids Res 47, 1082-1096 (2019).
4. M. F. Osborn, A. Khvorova, Improving siRNA Delivery In Vivo Through Lipid Conjugation. Nucleic Acid Ther 28, 128-136 (2018).
5. J. F. Alterman et al., A divalent siRNA chemical scaffold for potent and sustained modulation of gene expression throughout the central nervous system. Nat Biotechnol 37, 884-894 (2019).
6. V. N. Hariharan et al., Divalent siRNAs are bioavailable in the lung and efficiently block SARS-CoV-2 infection. Proc Natl Acad Sci USA 120, e2219523120 (2023).
7. R. A. Haraszti et al., 5′-Vinylphosphonate improves tissue accumulation and efficacy of conjugated siRNAs in vivo. Nucleic Acids Res 45, 7581-7592 (2017).
8. E. Elkayam et al., siRNA carrying an (E)-vinylphosphonate moiety at the 5′ end of the guide strand augments gene silencing by enhanced binding to human Argonaute-2. Nucleic Acids Res 45, 5008 (2017).
9. R. Parmar et al., 5′-(E)-Vinylphosphonate: A Stable Phosphate Mimic Can Improve the RNAi Activity of siRNA-GalNAc Conjugates. Chembiochem 17, 985-989 (2016).
10. C. Selvam, D. Mutisya, S. Prakash, K. Ranganna, R. Thilagavathi, Therapeutic potential of chemically modified siRNA: Recent trends. Chem Biol Drug Des 90, 665-678 (2017).
11. F. Eckstein, Phosphorothioates, essential components of therapeutic oligonucleotides. Nucleic Acid Ther 24, 374-387 (2014).
12. A. D. Springer, S. F. Dowdy, GalNAc-siRNA Conjugates: Leading the Way for Delivery of RNAi Therapeutics. Nucleic Acid Ther 28, 109-118 (2018).
13. A. Biscans et al., Docosanoic acid conjugation to siRNA enables functional and safe delivery to skeletal and cardiac muscles. Mol Ther 29, 1382-1394 (2021).
14. S. M. Davis et al., Chemical optimization of siRNA for safe and efficient silencing of placental sFLT1. Mol Ther Nucleic Acids 29, 135-149 (2022).
15. A. Sakowicz, M. Bralewska, M. Rybak-Krzyszkowska, M. Grzesiak, T. Pietrucha, New Ideas for the Prevention and Treatment of Preeclampsia and Their Molecular Inspirations. Int J Mol Sci 24 (2023).
16. B. Park (2023) Novel siRNA Therapy Fast Tracked for Preeclampsia Treatment. in MPR Monthly Prescribing Reference, p NA.
17. S. M. Hoy, Patisiran: First Global Approval. Drugs 78, 1625-1631 (2018).
18. L. J. Scott, Givosiran: First Approval. Drugs 80, 335-339 (2020).
19. L. J. Scott, S. J. Keam, Lumasiran: First Approval. Drugs 81, 277-282 (2021).
20. Y. N. Lamb, Inclisiran: First Approval. Drugs 81, 389-395 (2021).
21. A. Mullard, FDA approves fifth RNAi drug—Alnylam's next-gen hATTR treatment. Nat Rev Drug Discov 21, 548-549 (2022).
22. K. M. Brown et al., Expanding RNAi therapeutics to extrahepatic tissues with lipophilic conjugates. Nat Biotechnol 40, 1500-1508 (2022).
23. Q. Tang et al., Rational design of a JAK1-selective siRNA inhibitor for the modulation of autoimmunity in the skin. Nat Commun 14, 7099 (2023).
24. Q. Tang et al., RNAi-based modulation of IFN-gamma signaling in skin. Mol Ther 30, 2709-2721 (2022).
25. J. Gomez-Cambronero, Phospholipase D in cell signaling: from a myriad of cell functions to cancer growth and metastasis. J Biol Chem 289, 22557-22566 (2014).

1. A conjugated oligonucleotide of formula (I): 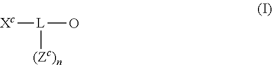
wherein:
O represents one or more oligonucleotides;
L represents a linker;
X^{c} represents a hydrophobic moiety; and
Z^{c} represents a headgroup having the structure of formula (II): 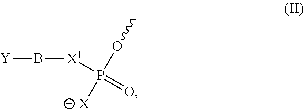
wherein:
Y is selected from the group consisting of —[NR₃]⁺, —[SR₂]⁺, —NR₂, and —CH(NH₂)—CO₂H, wherein each R is independently selected from the group consisting of hydrogen, and optionally substituted C1-C9 alkyl, C1-C9 heteroalkyl, C1-C9 alkenyl, C1-C9 heteroalkenyl, C1-C1 alkynyl, and C1-C9 heteroalkynyl groups;
B is a bridge group selected from the group consisting of substituted or unsubstituted C2-C9 alkyl, C2-C9 heteroalkyl, C2-C9 alkenyl, C2-C9 heteroalkenyl, C2-C9 alkynyl, and C2-C9 heteroalkynyl groups;
X¹ is absent, an oxygen atom, or NH;
X is oxygen, sulfur, or borane; and
n is 1 or 2, and
wherein when n is 1, Z^{c} is not phosphocholine, phosphoserine, or phosphoethanolamine.

2. The conjugated oligonucleotide of claim 1, wherein 13 includes one or more substituents independently selected from the group consisting of an alkyl, a substituted alkyl, an alkenyl, a substituted alkenyl, a halides, —OR′, —NR′R″, —CF₃, —CN, —NO₂, —C₂R′, —SR′, —N₃, —C(═O)NR′R″, —NR′C(O)R″, —C(═O)R′—C(═O)OR′—OC(O)R′, —OC(O)NR′R″, —NR′C(O)OR″, —SO₂R′, —SO₂NR′R″, and —NR′SO₂R″, wherein R′ and R″ are individually hydrogen or C1-C10 alkyl groups.

3. The conjugated oligonucleotide of claim 1, wherein Y is represented by formula (III) 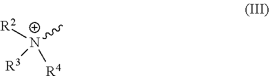
wherein R², R³, and R⁴ are independently selected from a hydrogen atom and substituted or unsubstituted C1-C10 alkyl, alkenyl, alkynyl, heteroalkyl, heteroalkenyl, or heteroalkynyl groups, wherein each heteroatom is independently selected from oxygen, nitrogen, silicon, sulfur, and halogen atoms.

4. The conjugated oligonucleotide of claim 1, wherein substituents for R², R³, and R⁴ are independently selected from the group consisting of an alkyl, a substituted alkyl, an alkenyl, a substituted alkenyl, halides, —OR′, —NR′R″, —CF₃, —CN, —NO₂, —C₂R′, —SR′, —N₃, —C(═O)NR′R″, —NR′C(O)R″, —C(═O)R′—C(═O)OR′—OC(O)R′, —O(CR′R″)ᵣC(═O)R′, —O(CR′R″)ᵣNR″C(O)R′, —O(CR′R″)ᵣNR″SO₂R′, —OC(O)NR′R″, —NR′C(O)OR″, —SO₂R′, —SO₂NR′R″, and —NR′SO₂R″, wherein R′ and R″ are individually hydrogen, an C1-C9 alkyl, a cycloalkyl, a heterocyclyl, an aryl, or an arylalkyl, and r is an integer from 1 to 6.

5. The conjugated oligonucleotide of claim 1, wherein R², R³, and R⁴ are independently a hydrogen atom or an unsubstituted C1-C9 alkyl group.

6. The conjugated oligonucleotide of claim 5, wherein R², R³, and R⁴ are methyl groups.

7. (canceled)

8. The conjugated oligonucleotide of claim 1, wherein L is connected to O via one or more branch points, and optionally one or more spacers, optionally:
wherein each of the one or more branch points is a polyvalent organic species or a derivative thereof; and each of the one or more spacers is independently selected from the group consisting of an ethylene glycol chain, an alkyl chain, a peptide, an RNA, a DNA, a phosphate, a phosphonate, a phosphoramidate, an ester, an amide, a triazole, and combinations thereof;
wherein the one or more branch points are selected from the group consisting of triols, tetrols, tri-carboxylic acids, tetra-carboxylic acids, tertiary amines, triamines, tetramines, and amino acids; and/or
comprising 1 to 3 branch points.

9-15. (canceled)

16. The conjugated oligonucleotide of claim 1, wherein X^{c} comprises one or more of eicosapentaenoic acid (EPA), docosahexaenoic acid (DHA), cholesterol, myristic acid, palmitic acid, and docosanoic acid (DCA).

17-23. (canceled)

24. The conjugated oligonucleotide of claim 1, wherein the one or more oligonucleotides is independently selected from the group consisting of a DNA, a siRNA, a shRNA, an antagomiR, a miRNA, antisense molecules, gapmers, mixmers, and a guide RNA.

25-29. (canceled)

30. The conjugated oligonucleotide of claim 1, wherein at least one of the one or more oligonucleotides has complementarity to a target.

31-37. (canceled)

38. The conjugated oligonucleotide of claim 1, wherein O is one or more double-stranded oligonucleotides, each comprising a sense strand and an antisense strand, wherein each of the sense strand and the antisense strand has a 5′ end and a 3′ end.

39-47. (canceled)

48. The conjugated oligonucleotide of claim 38, wherein the one or more double-stranded oligonucleotides comprises at least one modified internucleotide linkage of formula (IV): 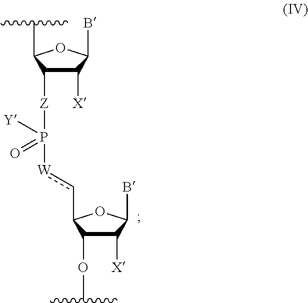
wherein:
B′ is a base pairing moiety;
W is selected from the group consisting of O, OCH₂, OCH, CH₂, and CH, or W is O or O(CH₂)ₙ;
X′ is selected from the group consisting of H, halo, hydroxy, OR, F, SH, SR, NR²₂ and a C1-C6-alkoxy;
Y′ is selected from the group consisting of O⁻, OH, OR, OR², NH⁻, NH₂, NR²₂, BH₃, S⁻, R¹, and SH; and
Z is selected from the group consisting of O, CH₂, or O(CH₂)ₙ,
wherein:
R is a protecting group;
R¹ is an alkyl, an allyl or an aryl;
R² is an alkyl, an allyl or an aryl;
n is an integer from 1 to 10; and [symbol]
is an optional double bond.

49-52. (canceled)

53. The conjugated oligonucleotide of claim 38, wherein:
(1) the antisense strand comprises at least 16 contiguous nucleotides, a 5′ end, a 3′ end and has complementarity to a target;
(2) the sense strand comprises at least 15 contiguous nucleotides, a 5′ end, a 3′ end, and has homology with a target; and
(3) a portion of the antisense strand is complementary to a portion of the sense strand.

54-56. (canceled)

57. The conjugated oligonucleotide of claim 38, wherein:
(1) the antisense strand comprises alternating 2′-methoxy-ribonucleotides and 2′-fluoro-ribonucleotides, wherein each nucleotide is a 2′-methoxy-ribonucleotide or a 2′-fluoro-ribonucleotide; and the nucleotides at positions 2 and 14 from the 5′ end of the antisense strand are not 2′-methoxy-ribonucleotides;
(2) the sense strand comprises alternating 2′-methoxy-ribonucleotides and 2′-fluoro-ribonucleotides, wherein each nucleotide is a 2′-methoxy-ribonucleotide or a 2′-fluoro-ribonucleotide; and the nucleotides at positions 2 and 14 from the 5′ end of the sense strand are 2′-methoxy-ribonucleotides;
(3) the nucleotides of the antisense strand are connected to adjacent nucleotides via phosphodiester or phosphorothioate linkages, wherein the nucleotides at positions 1-6 from the 3′ end, or positions 1-7 from the 3′ end are connected to adjacent nucleotides via phosphorothioate linkages; and
(4) the nucleotides of the sense strand are connected to adjacent nucleotides via phosphodiester or phosphorothioate linkages, wherein the nucleotides at positions 1 and 2 from the 3′ end are connected to adjacent nucleotides via phosphorothioate linkages.

58-62. (canceled)

63. The conjugated oligonucleotide of claim 53, wherein:
the antisense strand has perfect complementarity to the target;
the sense strand has complete homology with the target; and/or
the target is mammalian or viral mRNA, optionally wherein the target is an intronic region of the mammalian or viral mRNA.

64-67. (canceled)

68. The conjugated oligonucleotide of claim 1, wherein L is selected from the group consisting of: 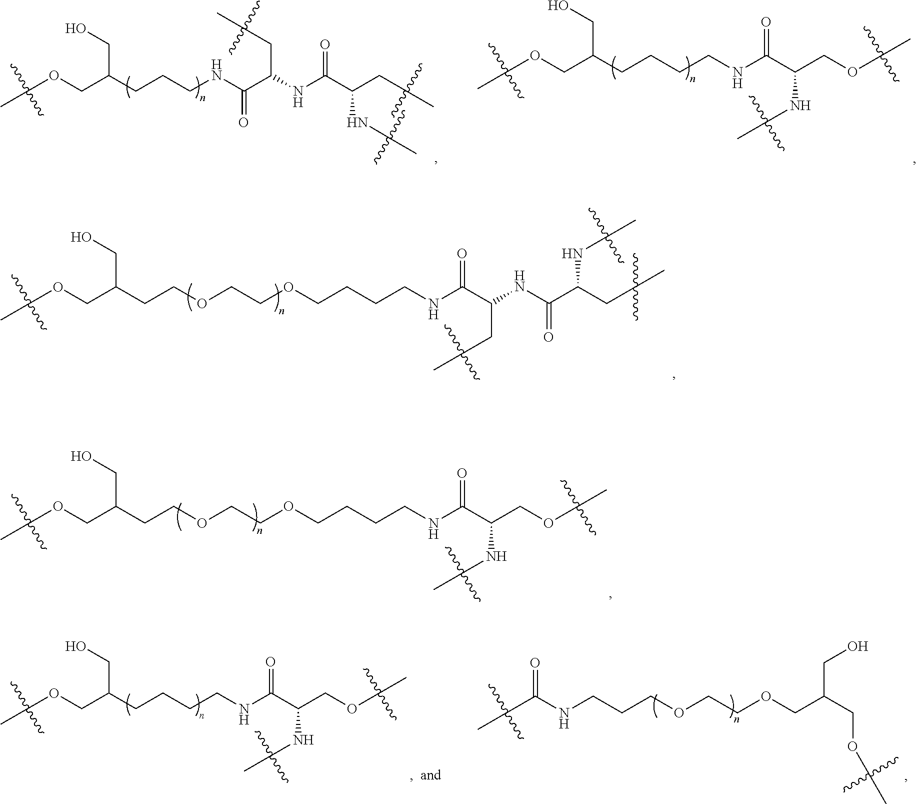
wherein n is 0, 1, 2, 3, 4, or 5.

69. The conjugated oligonucleotide of claim 1, wherein:
n is 1 and L is: 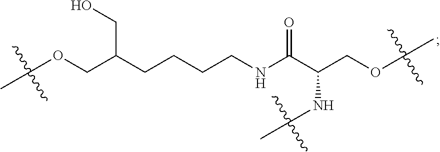 [SMILES: *N[C@@H](CO*)C(=O)NCCCCC(CO)CO*]
n is 2 and L is:

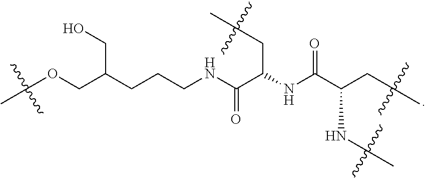

70. (canceled)

71. The conjugated oligonucleotide of claim 1, wherein:
O is a siRNA;
L is 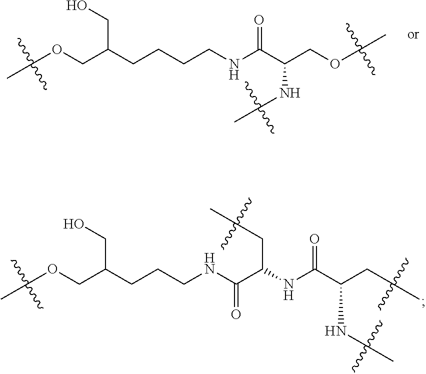
X^{c} is DCA;
and Z^{c} is: 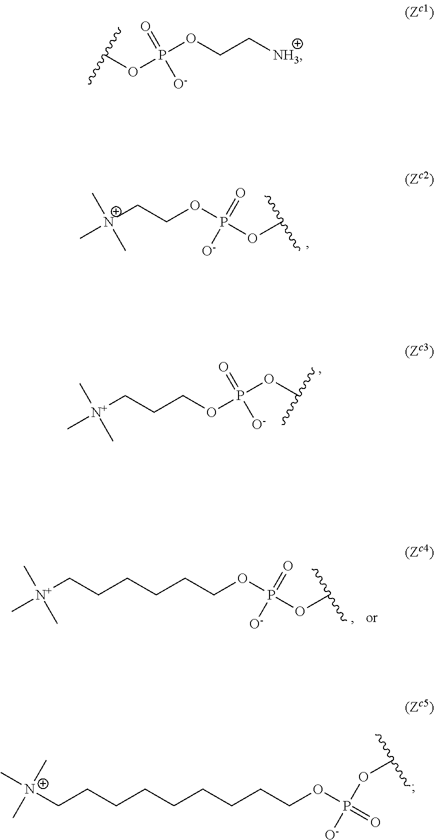
n is 1 or 2, and
wherein when n is 1, Z^{c} is not Z^{c1} or Z^{c2}.

72-73. (canceled)

74. A pharmaceutical composition comprising a therapeutically effective amount of one or more conjugated oligonucleotides according to claim 1; and a pharmaceutically acceptable carrier.

75. A method for selectively delivering the conjugated oligonucleotide of claim 1 to an organ in a patient, comprising administering the conjugated oligonucleotide to the patient, wherein the conjugated oligonucleotide selectively accumulates in one or more of heart, kidneys, muscle, lung, urinary bladder, pancreas, duodenum, spleen, adrenal gland, reproductive, extra-embryonic, and fat tissue of the patient, optionally in at least heart, muscle, or lung.

76. A method for tissue-specific silencing of a therapeutic target in a subject in need thereof, comprising administering the conjugated oligonucleotide of claim 1 to the subject, whereby the conjugated oligonucleotide selectively accumulates in one or more of heart, kidneys, muscle, lung, urinary bladder, pancreas, duodenum, spleen, adrenal gland, reproductive, extra-embryonic, and fat tissue in the subject, optionally in at least heart, muscle, or lung.

77. A multivalent conjugated oligonucleotide of formula (I_{A}): 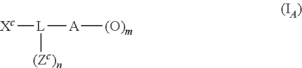
wherein:
O represents an oligonucleotide;
m is at least two;
L represents a linker;
A represents one or more branching points, one or more spacers, or a combination thereof;
X^{c} represents a hydrophobic moiety; and
Z^{c} represents a headgroup represented by formula (II): 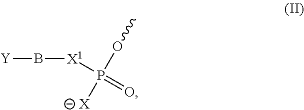
wherein:
Y is selected from the group consisting of —[NR₃]⁺, —[SR₂]⁺, —NR₂, and —CH(NH₂)−CO₂H, wherein each R is independently selected from the group consisting of hydrogen, and optionally substituted C1-C9 alkyl, C1-C9 heteroalkyl, C1-C9 alkenyl, C1-C9 heteroalkenyl, C1-C1 alkynyl, and C1-C9 heteroalkynyl groups;
B is a bridge group selected from the group consisting of substituted or unsubstituted C2-C9 alkyl, C2-C9 heteroalkyl, C2-C9 alkenyl, C2-C9 heteroalkenyl, C2-C9 alkynyl, and C2-C9 heteroalkynyl groups;
X¹ is absent, an oxygen atom, or NH;
X is oxygen, sulfur, or borane; and
n is 1 or 2,
wherein when n is 1, Z^{c} is not phosphocholine, phosphoserine, or phosphoethanolamine.

78-141. (canceled)

142. The conjugated oligonucleotide of claim 77, wherein:
the oligonucleotide is a double stranded oligonucleotide comprising a sense strand and an antisense strand, wherein the sense strand and the antisense strand each have a 5′ end and a 3′ end;
wherein the antisense strand comprises at least 16 contiguous nucleotides and has complementarity to a target, the sense strand comprises at least 15 contiguous nucleotides and has homology with a target; and a portion of the antisense strand is complementary to a portion of the sense strand; and wherein:
the antisense strand has perfect complementarity to the target;
the sense strand has complete homology with the target; and/or
the target is mammalian or viral mRNA, optionally wherein the target is an intronic region of the mammalian or viral mRNA.

143-146. (canceled)

147. A pharmaceutical composition comprising a therapeutically effective amount of one or more multivalent conjugated oligonucleotides according to claim 77; and a pharmaceutically acceptable carrier.

148. A method for selectively delivering the multivalent conjugated oligonucleotide of claim 77 to an organ in a patient, comprising administering the multivalent conjugated oligonucleotide to the patient, wherein the multivalent conjugated oligonucleotide selectively accumulates in one or more of heart, kidneys, muscle, lung, urinary bladder, pancreas, duodenum, spleen, adrenal gland, reproductive, extra-embryonic, and fat tissue of the patient, optionally in at least heart, muscle, or lung.

149. A method for tissue-specific silencing of a therapeutic target in a subject in need thereof, comprising administering the multivalent conjugated oligonucleotide of claim 77 to the subject, whereby the conjugated oligonucleotide selectively accumulates in one or more of heart, kidneys, muscle, lung, urinary bladder, pancreas, duodenum, spleen, adrenal gland, reproductive, extra-embryonic and fat tissue in the subject, optionally in at least heart, muscle, or lung.
